(19) United States
(12) Patent Application Publication
(10) Pub. No.: US 2024/0150836 A1
(43) Pub. Date: May 9, 2024

(54) METHODS OF PREDICTING AND TREATING IMMUNOTHERAPY TOXICITY BASED ON IMMUNE CELL POPULATIONS

(71) Applicant: THE BOARD OF REGENTS OF THE UNIVERSITY OF TEXAS SYSTEM, Austin, TX (US)

(72) Inventors: Jason Y. Park, Dallas, TX (US); John David Farrar, Dallas, TX (US); David E. Gerber, Dallas, TX (US); Farjana Fattah, Carrollton, TX (US); Hong Mu-Mosley, Dallas, TX (US)

(73) Assignee: THE BOARD OF REGENTS OF THE UNIVERSITY OF TEXAS SYSTEM, Austin, TX (US)

(21) Appl. No.: 18/504,868

(22) Filed: Nov. 8, 2023

Related U.S. Application Data
(60) Provisional application No. 63/382,972, filed on Nov. 9, 2022.
(60) Provisional application No. 63/503,946, filed on May 23, 2023.

(51) Int. Cl. C12Q 1/6883; G01N 33/564; G01N 33/569; G01N 33/68

(52) U.S. Cl. CPC C12Q 1/6883; G01N 33/564; G01N 33/56972; G01N 33/6863; C12Q 2600/118; C12Q 2600/158; G01N 2800/24

(57) ABSTRACT

The present disclosure generally relates to compositions and methods for predicting or diagnosing immune-related adverse events (irAE) before, during, or after immune checkpoint inhibitor (ICI) treatment in a subject with cancer. The method includes assessment of transcripts, autoantibody levels, cytokine levels, and immune cells. The irAE can be ICI-related myositis, ICI-related myocarditis or ICI-related myositis and myocarditis.

### CROSS REFERENCE TO RELATED APPLICATIONS

The present application claims the benefit of U.S. Provisional Patent Application No. 63/382,972, entitled, “METHODS OF PREDICTING AND TREATING IMMUNOTHERAPY TOXICITY BASED ON BIOMARKERS INCLUDING RNA” filed Nov. 9, 2022, and U.S. Provisional Patent Application No. 63/503,946, entitled, “METHODS OF PREDICTING AND TREATING IMMUNOTHERAPY TOXICITY BASED ON BIOMARKERS INCLUDING RNA” filed May 23, 2023. The contents of which are hereby incorporated by reference in their entireties.

### ACKNOWLEDGEMENT OF GOVERNMENT SUPPORT

This invention was made with support under Grant Nos. A1156189 and CA201543 awarded by the National Institutes of Health (NIH). The government has certain rights in the invention.

### BACKGROUND

### 1. Field

The present disclosure relates to identification of biomarkers for predicting, diagnosing, or monitoring immune-related adverse events associated with immune checkpoint inhibitor therapy.

### 2. Background

Immune-related adverse events (irAE) may affect almost any organ system during and after treatment with immune checkpoint inhibitors (ICI). ICI-related myositis and myocarditis are rare but potentially lethal toxicities. Understanding the etiology of these cases and their pathophysiologic differentiation from non-ICI-related inflammatory myopathies and myocarditis is critical to optimal monitoring and treatment of patients receiving ICI.

### SUMMARY

In some aspects, the disclosure provides a method of predicting the risk of developing and/or diagnosing immune-related adverse events (irAE) associated with immune checkpoint inhibitor (ICI) treatment in a subject comprising, providing a sample from the subject, assessing one or more transcript levels in the sample, and predicting risk for developing/diagnosing irAE in the subject wherein, the subject is predicted as having a high risk of developing or diagnosed with having irAE if the transcript levels of one or more of leukocyte immunoglobulin like receptor B4 (LILRB4), cytokine inducible SH2 containing protein (CISH), poly(ADP-ribose) polymerase family member 9 (PARP9), ring finger protein 145 (RNF145), asialoglycoprotein receptor 2 (ASGR2), solute carrier family 16 member 13 (SLC16A13), lysophosphatidic acid receptor 6 (LPAR6), GTPase, IMAP family member 7 (GIMAP7), C-X-C motif chemokine receptor 6 (CXCR6), dehydrogenase/reductase 9 (DHRS9), Fc gamma receptor Ic, pseudogene (FCGR1CP), and/or ankyrin repeat domain 34B (ANKRD34B) is elevated prior to ICI treatment (baseline), compared to a control sample; and/or if the transcript levels of one or more of Amphiregulin (AREG), epiregulin (EREG), Oncostatin M (OSM), cysteine and serine rich nuclear protein 1 (CSRNP1), DNA damage inducible transcript 4 (DDIT4), IL-10 (interleukin 10), Prostaglandin-endoperoxide synthase (PTGS2), Dual Specificity Phosphatase 1 (DUSP1), C-X-C chemokine receptor type 4 (CXCR4), Nuclear Factor, Interleukin 3 Regulated (NFIL3), Fos proto-oncogene, AP-1 transcription factor subunit (FOS), NFKB inhibitor alpha (NFKBIA), PPP1R15A (protein phosphatase 1 regulatory subunit 15A), CD79A, JunB proto-oncogene, AP-1 transcription factor subunit (JUNB), C-X-C motif chemokine ligand 8 (CXCL8), Early growth response 1 (EGR1), G0/G1 switch 2 (G0S2), paired box 8 (PAX8), activating transcription factor 6 beta (ATF6B), PAX8 antisense RNA1 (PAX8-AS1), RNA, variant U1 small nuclear 19 (RNVU1-19), vitelline membrane outer layer 1 homolog (VMO1), heparin binding EGF like growth factor (HBEGF), coiled-coil domain containing 144A (CCDC144A), shisa family member 8 (SHISA8), nuclear receptor subfamily 4 group A member 2 (NR4A2), prostaglandin E synthase (PTGES), synapsin I (SYN1), C-X-C motif chemokine ligand 2 (CXCL2), Peripheral myelin protein 22 (PMP22), CD83, early growth response 3 (EGR3), NUAK family kinase 1 (NUAK1), nocturnin (NOCT), atonal bHLH transcription factor 8 (ATOH8), polo like kinase (PLK2), inhibitor of DNA binding 1 (ID1), adrenoceptor beta 1 (ADRB1), snail family transcriptional repressor 1 (SNAIL), notch receptor 3 (NOTCH3), activating transcription factor 3 (ATF3), dual specificity phosphatase 2 (DUSP2), period circadian regulator 1 (PER1), TNF superfamily member 9 (TNFSF9), MAF bzip transcription factor F (MAFF), microRNA 4420 (MIR4420), glutathione peroxidase (GPX3), TNF alpha induced protein 3 (TNFAIP3), potassium voltage-gated channel modifier subfamily G member 1 (KCNG1), prostaglandin-endoperoxidase synthase 2 (PTGS2), A-kinase anchoring protein 5 (AKAP5), dual specificity phosphatase 1 (DUSP1), diacylglycerol kinase kappa (DGKK), beta-1,4,-N-acetyl-galactosaminyltransferase 3 (B4GALNT3), tribbles pseudokinase 1 (TRIB1), phorobol-12-myristate-13-acetate-induced protein 1 (PMAIP1), C-X-C motif chemokine receptor 4 (CXCR4), tumor protein p53 inducible nuclear protein 2 (TP53INP2), nuclear factor, interleukin 3 regulated (NFIL3), dual specificity phosphatase 4 (DUSP4), NFKB inhibitor alpha (NFKBIA), arginine vasopressin induced 1 (AVPI1), CD79a, ADP ribosylation factor like GTPase 4D (ARL4D), joining chain of multimeric IgA and IgM (JCHAIN), BTG anti-proliferation factor 2 (BTG2), TLE family member 1, transcriptional corepressor (TLE1), nuclear transport factor 2 like export factor 1 (NXT1), transducer of ERBB2, 1 (TOB1), phosphodiesterase 4D (PDE4D), DNAJ heat shock protein family member B1 (DNAJB1), AT-rich interaction domain 5B (ARID5B), G protein-coupled receptor 153 (GPR153), KLF transcription factor 9 (KLF9), SBDS ribosome maturation factor (SBDS), immediate early response 2 (IER2), TSC22 domain family member 3 (TSC22D3), GABA type A receptor associated protein like 1 (GABARAPL1), JunD proto-oncogene, AP-1 transcription factor subunit (JUND), RUNX family transcription factor 3 (RUNX3), BABAM2 antisense RNA 1 (BRE-AS1), putative salt inducible kinas 1B (LOC102724428), FAM46C (FAM46C), and/or general receptor for phosphoinositides 1-associated scaffold protein (GRASP) are lower in the subject when compared to the transcript levels in a control sample.

Further provided is a method of monitoring the risk of developing irAE associated with ICI treatment in a subject comprising, providing a sample from the subject, assessing one or more transcript levels in the sample, and monitoring risk for developing irAE in the subject wherein, the subject is predicted as having a high risk of developing irAE if the transcript levels of one or more of LILRB4, CISH, PARP9, RNF145, ASGR2, SLC16A13, LPAR6, GIMAP7, CXCR6, DHRS9, ANKRD34B, and/or FCGR1CP are elevated prior to ICI treatment (baseline), compared to a the transcript levels in a control sample; and/or the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are lower in the sample from the subject when compared to the transcript levels in a control sample.

In some aspects, the irAE comprises ICI-related myositis, ICI-related myocarditis, or ICI-related myositis and myocarditis.

In some aspects of the method, the assessment of transcript levels is performed before ICI treatment.

In some aspects, the subject is predicted as high risk of developing irAE if the transcript levels of one or more of LILRB4, CISH, and/or PARP9 are elevated prior to ICI treatment (baseline), compared to the transcript levels in a control sample.

In some aspects, the subject is predicted as high risk of developing irAE if the transcript levels of one or more of LILRB4, CISH, GIMAP7, CXCR6, DHRS9, ANKRD34B, and/or FCGR1CP are elevated prior to ICI treatment (baseline), compared to the transcript levels in a control sample.

In some aspects, the subject is predicted as high risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP are lower prior to ICI treatment, compared to the transcript levels in a control sample.

In some aspects, the subject is predicted as high risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are lower prior to ICI treatment, compared to the transcript levels in a control sample.

In some aspects, the subject is predicted as high risk of developing irAE if the transcript levels of one or more of AREG, EREG, CXCL8, EGR1, G0S2, PAX8, ATF6B, PAX8-AS1, RNVU1-19, VMO1, HBEGF, CCDC144A, and/or SHISA8, are lower prior to ICI treatment, compared to the transcript levels in a control sample.

In some aspects, the subject is predicted as high risk of developing irAE if the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower prior to ICI treatment, compared to the transcript levels in a control sample.

In some aspects, the subject is predicted as having a high risk of developing irAE if the transcript level of PARP9 is elevated prior to ICI treatment (baseline), compared to the transcript level in a control sample; and/or the transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are lower prior to ICI treatment (baseline), compared to the transcript levels in a control sample.

In some aspects, the subject is predicted as high risk for irAE if the transcript levels of one or more of PARP9, CISH, CXCR6, LPAR6, and/or ASGR2 are elevated prior to ICI treatment (baseline), compared to the transcript levels in a control sample; and/or the transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1 are lower prior to ICI treatment (baseline), compared to the transcript levels in a control sample.

In some aspects, the sample is whole blood, serum, plasma, cerebrospinal fluid, pleural fluid, pericardial fluid, peritoneal fluid, bone marrow, or tissue, urine, cerebrospinal fluid (CSF), or other body fluid

In some aspects, the said ICI treatment is administered as part of cancer treatment.

In some aspects, the said ICI treatment comprises administration of an inhibitor of PD-1, PD-L1, TIM-3, LAG-3, CTLA-4, CSF-1R, or any combinations thereof.

In some aspects, assessing transcript levels (step b) comprises RNA-seq, Nanopore sequencing, Nanostring, multiplex RT-PCR, single-plex RT-PCR, NASBA, Fluorescence measurements or spectrophotometry.

In some aspects, the method further comprises assessing the expression of one or more of Mi-2, GAD65, Myosin, Thyroglobulin and/or TPO in the sample from the subject.

In some aspects, the assessment comprises identifying if the expression of one or more autoantibodies are elevated in the sample from the subject compared to the abundance in a control sample.

In some aspects, the method further comprises assessing expression of one or more of CXCL2, CXCL5, CXCL6, CCL7, CCL19, IFNγ, IL-6, CXCL9 and/or CXCL10 in the sample from the subject.

In some aspects, the assessment comprises identifying if the expression of one or more cytokines are elevated in the sample from the subject compared to the expression in a control sample.

In some aspects, the method further comprises assessing abundance of one or more of PD-L+ naive B cells, switched memory B cells, and/or CTLA-4+ monocytes in the sample from the subject.

In some aspects, the assessment comprises identifying if the abundance of one or more PD-L+ naive B cells, and/or switched memory B cells are decreased, and/or if the abundance of CTLA-4+ monocyte is elevated, in the sample from the subject compared to the abundance in a control sample.

In some aspects, the method further comprises repeating steps (a)-(c) at a second time point, thereby permitting determination of a change in the subject's risk of developing irAE and/or diagnosis of irAE in the sample from the subject compared to a control sample.

In some aspects, the method further comprises predicting the subject as having low risk if the transcript levels of one or more of LILRB4, CISH, PARP9, RNF145, ASGR2, SLC16A13, LPAR6, GIMAP7, CXCR6, DHRS9, ANKRD34B, and/or FCGR1CP prior to ICI treatment (baseline) is lower or equivalent compared to the transcript levels in a control sample; and/or the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are elevated or equivalent during ICI treatment, compared to the transcript levels in a control sample.

In some aspects, the method further comprises treating the subject with an ICI therapy when the subject is predicted to have a low risk for developing irAE.

In some aspects, the method further comprises treating the subject predicted as having a high risk of developing irAE with a non-ICI therapy or treating said subject with a ICI therapy and an irAE mitigating therapy, wherein the irAE mitigating therapy is selected from corticosteroids (e.g., prednisone, methylprednisolone, dexamethasone, budesonide), TNF inhibitors (e.g., infliximab), or hormone replacement (e.g., hydrocortisone, levothyroxine), CXCL8 inhibitors (e.g., repertaxin), or any combination thereof.

In some aspects, the disclosure further comprises a method of treating a subject with cancer comprising, (a) providing a sample from the subject, (b) assessing one or more transcript levels in the sample, (c) predicting the subject's risk of developing irAE, wherein the subject is diagnosed as: low risk when the transcript levels of one or more of LILRB4, CISH, PARP9, RNF145, ASGR2, SLC16A13, LPAR6, GIMAP7, CXCR6, DHRS9, ANKRD34B, and/or FCGR1CP are lower or equal to the transcript levels in a control sample; low risk when the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are elevated or equal to the transcript levels in a control sample; high risk when the transcript levels of one or more of LILRB4, CISH, PARP9, RNF145, ASGR2, SLC16A13, LPAR6, GIMAP7, CXCR6, DHRS9, ANKRD34B, and/or FCGR1CP are elevated than transcript levels in a control subject with no irAE or a healthy subject; and/or high risk when the transcript levels of one or more transcript of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are lower than the transcript levels in a control sample; (d) treating the subject with: (i) an ICI therapy if subject is diagnosed as low risk of developing irAE, (ii) a non-ICI therapy if the subject is diagnosed as high risk of developing irAE; or (iii) an ICI therapy and an irAE mitigating therapy if the subject is diagnosed as high risk of developing irAE.

In some aspects, irAE comprises ICI-related myositis, ICI-related myocarditis or ICI-related myositis and myocarditis.

In some aspects, the subject is predicted as high risk of developing irAE if the transcript levels of one or more of LILRB4, CISH, and/or PARP9 are elevated prior to ICI treatment (baseline), compared to the transcript levels in a control sample.

In some aspects, the subject is predicted as high risk of developing irAE if the transcript levels of one or more of LILRB4, CISH, GIMAP7, CXCR6, DHRS9, ANKRD34B, and/or FCGR1CP is elevated prior to ICI treatment (baseline), compared to the transcript levels in a control sample.

In some aspects, the subject is predicted as high risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP are lower prior to ICI treatment, compared to the transcript levels in a control sample.

In some aspects, the subject is predicted as high risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are lower prior to ICI treatment, compared to the transcript levels in a control sample.

In some aspects, the subject is predicted as high risk of developing irAE if the transcript levels of one or more of AREG, EREG, CXCL8, EGR1, G0S2, PAX8, ATF6B, PAX8-AS1, RNVU1-19, VMO1, HBEGF, CCDC144A, and/or SHISA8, are lower prior ICI treatment, compared to the transcript levels in a control sample.

In some aspects, the subject is predicted as high risk of developing irAE if the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower prior to ICI treatment, compared to the transcript levels in a control sample.

In some aspects, the subject is predicted as high risk for irAE if the transcript level of PARP9 is elevated prior to ICI treatment (baseline), compared to the transcript level a control sample; and/or the transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are lower prior to ICI treatment (baseline), compared to the transcript levels in a control sample.

In some aspects, the subject is predicted as high risk for irAE if the transcript levels of one or more of PARP9, CISH, CXCR6, LPAR6, and/or ASGR2 are elevated prior to ICI treatment (baseline), compared to transcript levels in a control sample; and/or the transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1 are lower prior to ICI treatment (baseline), compared to the transcript levels in a control sample.

In some aspects, the assessment further comprises detecting the expression of one or more of CXCL2, CXCL5, CXCL6, CCL7, CCL19, IFNγ, IL-6, CXCL9 and/or CXCL10 in the sample from the subject.

In some aspects, the assessment comprises identifying if the expression of one or more cytokines are elevated in the sample from the subject compared to the expression in a control sample.

In some aspects, the assessment further comprises detecting the expression of one or more of Mi-2, GAD65, Myosin, Thyroglobulin and/or TPO in the sample from the subject.

In some aspects, the assessment comprises identifying if the expression of one or more autoantibodies are elevated in the sample from the subject compared to the expression in a control sample.

In some aspects, the method further comprises assessing the abundance of one or more of PD-L+ naive B cells, switched memory B cells, and/or CTLA-4+ monocytes in the sample from the subject.

In some aspects, the assessment comprises identifying if the abundance of one or more PD-L+ naive B cells, and/or switched memory B cells are decreased, and/or if the abundance of CTLA-4+ monocytes is elevated in the sample from the subject compared to the abundance in a control sample.

In some aspects, the ICI treatment comprises administration of an inhibitor of PD-1, PD-L1, TIM-3, LAG-3, CTLA-4, CSF-1R, or any combinations thereof.

Further provided herein is a method of identifying the presence of at least one differentially expressed transcript associated with irAE in a biological sample of a subject with cancer, the method comprising, providing a sample from the subject, assessing the transcript levels in the sample, wherein the assessment comprises detecting if the transcript levels of one or more of LILRB4, CISH, PARP9, RNF145, ASGR2, SLC16A13, LPAR6, GIMAP7, CXCR6, DHRS9, ANKRD34B, and/or FCGR1CP are elevated than the transcript levels in a control sample; the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are lower than the transcript levels in a control sample.

In some aspects, the subject is planning to undergo immune checkpoint inhibitor (ICI) treatment.

In some aspects, irAE comprises ICI-related myositis, ICI-related myocarditis or ICI-related myositis and myocarditis.

In some aspects, the method further comprises detecting the expression of one or more CXCL2, CXCL5, CXCL6, CCL7, CCL19, IFNγ, IL-6, CXCL9 and/or CXCL10 in the sample from the subject.

In some aspects, the assessment comprises identifying if the expression of one or more cytokines are elevated in the sample from the subject compared to the expression in a control sample.

In some aspects, the method further comprises detecting the expression of one or more of Mi-2, GAD65, Myosin, Thyroglobulin and/or TPO in the sample from the subject.

In some aspects, the assessment comprises identifying if the expression of one or more autoantibodies are elevated in the sample from the subject compared to the expression in a control sample.

In some aspects, the method further comprises assessing the abundance of one or more of PD-L+ naive B cells, switched memory B cells, and/or CTLA-4+ monocytes in the sample from the subject.

In some aspects, the assessment comprises identifying if the abundance of one or more of PD-L+ naive B cells, and/or switched memory B cells are decreased, and/or if the abundance of CTLA-4+ monocyte is elevated in in the sample from the subject compared to the abundance in a control sample.

In some aspects, the assessment comprises determining a baseline or a pre-treatment profile that correlates with future toxicity.

In some aspects, the baseline or a pre-treatment profile comprises the elevated transcript levels of one or more of LILRB4, CISH, and/or PARP9 compared to the transcript levels in a control sample.

In some aspects, the baseline or a pre-treatment profile comprises the elevated transcript levels of one or more of more of LILRB4, CISH, GIMAP7, CXCR6, DHRS9, ANKRD34B, and/or FCGR1CP compared to the transcript levels in a control sample.

In some aspects, the baseline or a pre-treatment profile comprises lower transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP, compared to the transcript levels in a control sample.

In some aspects, the baseline or a pre-treatment profile comprises lower transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB, compared to the transcript levels in a control sample.

In some aspects, the baseline or a pre-treatment profile comprises lower transcript levels of one or more of AREG, EREG, CXCL8, EGR1, G0S2, PAX8, ATF6B, PAX8-AS1, RNVU1-19, VMO1, HBEGF, CCDC144A, and/or SHISA8, compared to the transcript levels in a control sample.

In some aspects, the baseline or a pre-treatment profile comprises lower transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9, compared to the transcript levels in a control sample.

In some aspects, the baseline or a pre-treatment profile comprises, elevated transcript level of PARP9, compared to the transcript level in a control sample; and/or lower transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3, compared to the transcript levels in a control sample.

In some aspects, the baseline or a pre-treatment profile comprises, elevated transcript levels of one or more of PARP9, CISH, CXCR6, LPAR6, and/or ASGR2, compared to the transcript levels in a control sample; and/or lower transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1, compared to the transcript levels in a control sample.

In some aspects, the control sample is procured from a subject with a low risk of developing irAE.

In some aspects, the transcript levels of the disclosed methods are relative transcript levels.

### BRIEF DESCRIPTION OF THE DRAWINGS

The patent or application file contains at least one drawing executed in color. Copies of this patent or patent application with color drawing(s) will be provided by the Office by request and payment of the necessary fee.

FIG. 1 depicts contour plots displaying defining surface markers in clusters (immune cell subsets) for CyTOF analysis.

FIGS. 2A-2K show cytokine profiles in myositis/myocarditis cases and no toxicity controls. FIG. 2A depicts baseline cytokines. FIG. 2B-2H depict baseline cytokines with significant differences according to occurrence and grade of myositis/myocarditis. FIG. 2I illustrates cytokine changes after ICI initiation with significant differences according to occurrence of myositis/myocarditis. FIG. 2J-2K illustrate cytokine changes after ICI initiation with significant differences according to occurrence and grade of myositis/myocarditis. ICI, immune checkpoint inhibitor; irAE, immune-related adverse event; NT, no toxicity.

FIGS. 3A-3F depict autoantibody profiles in myositis/myocarditis cases and no toxicity controls. FIG. 3A shows baseline autoantibodies. FIG. 3B-3F show baseline autoantibodies with significant differences according to occurrence and grade of myositis/myocarditis.

FIGS. 4A-4C depict immune cell profiles in myositis/myocarditis cases and no toxicity controls. FIG. 4A illustrates using CyTOF, 28 clusters (immune cell subsets) were identified. The four immune cell subsets demonstrating significant differences between the irAE cases and no toxicity controls are shown. FIG. 4B shows that in pre-treatment baseline samples, irAE cases had reduced PD-L1+ naïve B cells (P=0.004), reduced switched memory B cells (P=0.03), and increased CTLA4+ monocytes (P=0.03) (Mann-Whitney test) compared to no toxicity controls. FIG. 4C illustrates after ICI initiation, irAE cases had greater increases in PD-L1+ naïve B cells (P=0.03), switched memory B cells (P=0.02), and CXCR3+CD8 T cells (P=0.01) (2-way ANOVA).

FIGS. 5A-5D depict transcription profiles in myositis/myocarditis cases (N=4) and no toxicity controls (N=3) using bulk RNA sequencing analysis. FIG. 5A shows principal component analysis (PCA) demonstrates differences in pre-treatment baseline transcription profiles according to irAE occurrence. FIG. 5B depicts volcano plot demonstrating differentially expressed genes (DEGs) between irAE cases and no toxicity controls in baseline samples. Ninety-four out of 14,174 genes had statistically significant differences, including 12 upregulated genes and 82 downregulated genes (false discover rate (FDR)<0.05). Among these, 7 upregulated (red) and 75 downregulated (blue) genes had |Log 2fold change(FC)|>1. FIG. 5C depicts heatmap of transcriptional profiles of irAE cases and no toxicity controls in baseline samples with 8 clusters showing differences. The top enriched term and FDR for each cluster from the GO_Biological_Process_2021 gene set library by gene otology (GO) analysis is shown in the right. FIG. 5D illustrates relative differences in expression between irAE cases and no toxicity controls for 18 (out of 94) genes associated with the gene sets in 8 functional biological processes.

FIG. 6 illustrates PCA plot of RNA-seq data from pre-treatment baseline and after ICI initiation in irAE cases and no toxicity cases. The drawing figures do not limit the present disclosure to the specific embodiments disclosed and described herein. The drawings are not necessarily to scale, emphasis instead being placed on clearly illustrating principles of certain embodiments of the present disclosure.

### DETAILED DESCRIPTION

The following detailed description references the accompanying drawings that illustrate various aspects of the present disclosure. The drawings and description are intended to describe aspects of the present disclosure in sufficient detail to enable those skilled in the art to practice the present disclosure. Other components can be utilized, and changes can be made without departing from the scope of the present disclosure. The following description is, therefore, not to be taken in a limiting sense.

Provided herein are methods of predicting, diagnosing and/or monitoring immune-related adverse events (irAE) in a subject undergoing or planning to undergo immune checkpoint inhibitor (ICI) treatment. The present disclosure is based on the surprising determination that subjects develop a unique transcript, autoantibody, cytokine, and/or immune cell profile at baseline or pre-treatment that correlates with irAE during ICI treatment, in subjects with cancer. These transcript, autoantibody, cytokine, and/or immune cell profiles can be used as biomarkers for predicting, diagnosing and/or monitoring irAE during ICI treatment and help guide more effective cancer treatment strategies with reduced toxic side effects, especially associated with ICI treatment.

### I. Terminology

For the purposes of promoting an understanding of the principles of the present disclosure, reference will now be made to preferred aspects and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some aspects, the subject can be a human. In other aspects, the subject can be a human in need of treating a cancer.

As used herein “immune-related adverse event” or “irAE” is diverse toxicities, side-effects or problems associated with cancer immunotherapy. Immunotherapy include therapy using immunotoxins, T-cell transfer, chimeric antigen receptors, antibodies, immune system modulators and immune checkpoint inhibitors, and/or other immunotherapies known to those of skill in the art.

As used herein “immune checkpoint inhibitor” is a drug that block immune checkpoints. These checkpoints are a normal part of the immune system and keep immune responses from being too strong. By blocking them, these drugs allow immune cells to respond more strongly for e.g., cancer. Immune checkpoint inhibitors work by preventing cancer cells from turning T-cells (white blood cells that detect infections and abnormalities) off. Non-limiting examples of immune checkpoint inhibitors include inhibitors of PD-1, PD-L1, TIM-3, LAG-3, CTLA-4, and CSF-1R and any combination thereof. The immune checkpoint receptors may be on tumor cells or immune cells such as T cells, monocytes, microglia, and macrophages, without limitation. The agents which assert immune checkpoint blockade may be small chemical entities or polymers, antibodies, antibody fragments, single chain antibodies or other antibody constructs, including, but not limited to, bispecific antibodies and diabodies. Immune checkpoint inhibitors which may be used according to the disclosure include any that disrupt the inhibitory interaction of cytotoxic T cells and tumor cells. These include but are not limited to anti-PD-1 antibody, anti-PD-L1 antibody, anti-CTLA4 antibody, anti-LAG-3 antibody, anti-TIM-3 antibody. The inhibitor need not be an antibody but can be a small molecule or other polymer. If the inhibitor is an antibody it can be a polyclonal, monoclonal, fragment, single chain, or other antibody variant construct. Inhibitors may target any immune checkpoint known in the art, including but not limited to, CTLA-4, PDL1, PDL2, PD1, B7-H3, B7-H4, BTLA, HVEM, TIM3, GAL9, LAG3, CSF-1R, VISTA, KIR, 2B4, CD160, CGEN-15049, CHK1, CHK2, A2aR, CD28, CD86, CD69, CD48, CD113, CEACAM-1, Galectin-1, TIGIT, GPR56, CD48, GARP, PD1H, LAIR1, TIM1, TIM4 and the B-7 family of ligands. Combinations of inhibitors for a single target immune checkpoint or different inhibitors for different immune checkpoints may be used. Illustrative examples of immune checkpoint inhibitors include CTLA-4 blocking antibodies (Ipilimumab (Yervoy), Tremelimumab (lmjuno)), PD-1 inhibitors (Pembrolizumab (Keytruda), Nivolumab (Opdivo), Cemiplimab (Libtayo), CT-011 (Pidilizumab), AMP224), PD-L1 inhibitors (Atezolizumab (tecentriq), Avelumab (Bavencio), Durvalumab (Imfinzi), BMS-936559), Lag3 inhibitors (Relatlimab), combination of Lag3 and PD1 inhibitor (PD-1 inhibitor nivolumab (Opdualag) OX40 inhibitor (MEDI6469), CD160 inhibitor (BY55). Non-limiting examples of inhibitors of CSF-1R include PLX3397, PLX486, RG7155, AMG820, ARRY-382, FPA008, IMC-CS4, JNJ-40346527, and MCS 110. The terms “ICI treatment”, “ICI therapy”, “ICI compounds”, and the like, refer to one or more ICI (or the use thereof) disclosed herein or known to those of skill in the art.

As used herein “transcript” or “RNA transcript” or “RNA” can be a messenger RNA (mRNA) molecule. In some aspects, RNA can be total RNA, mRNA, pre-mRNA, or any combination thereof.

As uses herein “autoantigen” is a normal protein or protein complex (and sometimes DNA or RNA) that is recognized by the immune system of patients suffering from a specific autoimmune disease. These antigens should not be, under normal conditions, the target of the immune system, but their associated T cells are not deleted and instead attack.

As used herein “cytokine” is a broad category of small proteins that are important in cell signaling. Release of cytokine has an effect on the behavior of cells around them. Cytokines are involved in autocrine signaling, paracrine signaling and endocrine signaling as immunomodulating molecules. Non-limiting examples of cytokines include chemokines, interferons, interleukins, lymphokines, and tumor necrosis factors. Cytokines are produced by a variety of cell types including immune cells like macrophages, monocytes, dendritic cells, B lymphocytes, T lymphocytes and mast cells, as well as endothelial cells, fibroblasts, and various stromal cells; and a given cytokine may be produced by more than one type of cell.

As used herein “immune cell” is a cell which develops from stem cells in the bone marrow and become different types of white blood cells. Immune cells include neutrophils, eosinophils, basophils, mast cells, monocytes, macrophages, dendritic cells, natural killer cells, and lymphocytes (B cells and T cells).

As used herein “abundance” refers to the amount of a particular analyte (e.g., immune cell subset) present in the sample. The amount may be a number, ratio, proportion, or a percentage of the analyte compared to the control sample or determined using a standard curve. The amount may be an absolute amount or a relative amount (e.g., relative to an internal control, etc.).

As used herein “expression” or “expression level” or “level of expression” refers to amount of a particular analyte (e.g., antibody or cytokine) present in the sample. The amount may be a concentration, number, ratio, proportion, or a percentage of the analyte compared to the control sample or determined using a standard curve. The amount may be an absolute amount or a relative amount.

As used herein “myositis” is the inflammation of the muscles help body move.

As used herein “myocarditis” is inflammation of the heart muscle.

As used herein “cancer” may be one or more neoplasm or cancer. The neoplasm may be malignant or benign, the cancer may be primary or metastatic; the neoplasm or cancer may be early stage or late stage. Non-limiting examples of neoplasms or cancers include acute lymphoblastic leukemia, acute myeloid leukemia, adrenocortical carcinoma, AIDS-related cancers, AIDS-related lymphoma, anal cancer, appendix cancer, astrocytoma (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, and Wilms tumor (childhood).

As used herein, treatment of cancer can comprise increased inhibition of cancer progression and/or metastases, inhibition of an increase in tumor volume, a reduction in tumor volume and/or growth, a reduction in tumor growth rate, an eradication of a tumor and/or cancer cell, or any combination thereof. In some aspects, the treatment can also prolong the survival of a subject, improve the prognosis and/or improve the quality of life of the subject.

As used herein, a biological sample may be of any biological tissue, fluid, or cell from the subject. The sample can be solid or fluid. The sample can be a heterogeneous cell population. Non-limiting examples of suitable biological samples include sputum, serum, blood, blood cells (e.g., white cells), a biopsy, urine, peritoneal fluid, pleural fluid, or cells derived therefrom. The biopsy can be a fine needle aspirate biopsy, a core needle biopsy, a vacuum assisted biopsy, an open surgical biopsy, a shave biopsy, a punch biopsy, an incisional biopsy, a curettage biopsy, or a deep shave biopsy. Biological samples may also include sections of tissues, such as frozen sections or formalin fixed sections taken for histological purposes. A sample can be a tumor tissue, tissue surrounding a tumor, or non-tumor tissue. Methods of collecting a biological sample from a subject are well known in the art. In some aspects, the biological sample is a peripheral blood sample. In some aspects, the biological sample is peripheral blood mononuclear cell (PBMC). In some aspects, the biological sample is plasma.

Sample from the subject can be procured one or more times, before, during and/or after diagnosis. In some aspects, samples can be procured from the subject before, during, and/or after treatment of cancer, wherein the cancer treatment comprises ICI treatment. In some aspects, sample can be procured from the subject prior to the start of ICI treatment. In some aspects, sample can be procured from the subject undergoing ICI treatment, before onset of irAE. In other aspects, sample can be procured after onset of irAE in a subject. In some aspects, sample can be procured before, during and/or after administration of a non-ICI cancer treatment, or ICI treatment combined with steroid treatment, for monitoring the treatment for irAE. Additionally, samples can be procured repeatedly at multiple stages after initial sample procurement, to determine and/or monitor irAE in a subject.

In some aspects, control sample can be procured from a healthy subject and/or a subject undergoing ICI treatment but has a low risk of developing irAE or ICI toxicity. In some aspects, the control sample can comprise non-cancer cells. In some aspects, the non-cancer cells can be from the same tissue type as the cancer cells. For example, if the cancer cells are from breast cancer, then the non-cancer cells can be from healthy breast tissue. In some aspects, the control can comprise an average levels of the biomarker profile in a sample from a subject before onset of cancer. In some aspects, control sample can be a sample from the subject prior to diagnosis or treatment. In certain aspects, the biomarker profile can be measured in a person or persons other than the subject with cancer. In some aspects, the control a person or persons with similar characteristics to the subject with cancer. In some aspects, the control can be an average of the combination of disclosed biomarker levels from different healthy sources (e.g., more than one healthy control subject and/or more than one subject has a low risk of developing irAE). In some aspects, the control sample can be pooled sample. In some aspects, the control sample is procured from a subject with low risk of developing irAE.

As used herein, a subject that has a low risk of developing irAE can be a subject or population that does not develop irAE with ICI treatment. In an aspect, a subject that has a low risk of developing irAE can be a subject or population that does not develop irAE with ICI treatment as determined through retrospective analysis to not develop irAE with ICI treatment.

### II. Biomarkers

The present disclosure provides immunological characteristics of ICI-related irAE of myositis and/or myocarditis and insights into the biological profiles of ICI-related myositis and/or myocarditis. These profiles can be used as biomarkers to provide rationales for potential cancer treatment options.

### Transcript Profiles

In some aspects, the present disclosure provides a method of predicting the risk of developing and/or diagnosing immune-related adverse events (irAE) associated with immune checkpoint inhibitor (ICI) treatment in a subject. The method comprises providing a sample from the subject, assessing transcript levels of one or more of transcripts in the sample; and predicting the risk for developing/diagnosing irAE in the subject. In some aspects, assessment of transcripts comprises comparing the transcript levels of one or more transcripts in the sample of the subject to the same transcript levels in a control sample. In some aspects, the transcript levels are relative transcript levels. In some aspects, transcript profile related to ICI-associated irAE comprises a transcript profile wherein transcript levels one or more transcripts are elevated in the subject compared to the transcript levels in a control sample. In some aspects, transcript profile related to ICI-associated irAE comprises a transcript profile wherein the transcript levels one or more transcripts are decreased in the subject compared to the transcript levels in a control sample.

In some aspects, the transcript is one or more of the transcripts disclosed in Table 5. In some aspects, the transcript is one or more of the transcripts disclosed in Table 6. In some aspects, the transcript is one or more of the transcripts disclosed in Table 8. In some aspects, the transcript is one or more of the transcripts disclosed in Table 9. In some aspects, the transcript is one or more of the transcripts disclosed in Table 10. In some aspects, the transcript is one or more of the transcripts disclosed in Table 11. In some aspects, transcript profile related to ICI-associated irAE comprises transcript levels of one or more transcripts with elevated level in the subject compared to the transcript levels in a control sample and transcript levels of one or more transcripts with decreased level in the subject compared to the transcript levels in a control sample. In some aspects, the transcript profile comprises one or more of the transcripts disclosed in Table 5, Table 6, Table 8, Table 9, Table 10, Table 11, or any combination thereof.

In some aspects, a subject is predicted or diagnosed as having high risk of developing ICI-associated irAE, when transcript levels of one or more transcripts are elevated in the subject compared to the transcript levels of the same transcript levels in a sample. In some aspects, a subject is predicted or diagnosed as having high risk of developing ICI-associated irAE, when transcript levels of one or transcripts are decreased in the subject compared to the transcript levels of the same transcript in a control sample. In other aspects, a subject is predicted or diagnosed as having low risk of developing ICI-associated irAE, when transcript levels of one or more transcripts are elevated in the subject compared to the transcript levels of the same transcripts in a control sample. In another aspect, a subject is predicted or diagnosed as having low risk of developing ICI-associated irAE, when transcript levels of one or more transcripts are decreased in the subject compared to the transcript levels of the same transcripts in a control sample.

In some aspects, the transcript profile comprises a baseline or a pre-treatment transcript profile that correlates with future toxicity. These transcripts are used as biomarkers to assess the risk of a subject developing irAE during ICI treatment. In some aspects, the subject is planning to undergo or is undergoing ICI treatment as part of a cancer therapy.

In some aspects, the transcript(s) having an elevated level of expression has an elevated expression level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater as compared to level of expression of the same transcript(s) in a control sample.

In some aspects, the transcript(s) having elevated level of expression has an elevated expression level having a log 2 fold change value from about 0.1 to about 5. For example, a log 2 fold change value can be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5.

In some aspects, the transcript(s) having decreased level of expression has a decreased expression level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least 100% lower as compared to level of expression of the same transcript(s) in a control sample.

In some aspects, the transcript having decreased level of expression has decreased expression level having a log 2 fold change value from about −0.1 to about −5. For example, a log 2 fold change value can be about −0.1, −0.2, −0.3, −0.4, −0.5, −0.6, −0.7, −0.8, −0.9, −1, −1.1, −1.2, −1.3, −1.4, −1.5, −1.6, −1.7, −1.8, −1.9, −2, −2.1, −2.2, −2.3, −2.4, −2.5, −2.6, −2.7, −2.8, −2.9, −3, −3.1, −3.2, −3.3, −3.4, −3.5, −3.6, −3.7, −3.8, −3.9, −4, −4.1, −4.2, −4.3, −4.4, −4.5, −4.6, −4.7, −4.8, −4.9, or −5.

In some aspects, a subject is predicted as having a high risk of developing or diagnosed with having irAE if the transcript levels of one or more of leukocyte immunoglobulin like receptor B4 (LILRB4), cytokine inducible SH2 containing protein (CISH), poly(ADP-ribose) polymerase family member 9 (PARP9), ring finger protein 145 (RNF145), asialoglycoprotein receptor 2 (ASGR2), solute carrier family 16 member 13 (SLC16A13), lysophosphatidic acid receptor 6 (LPAR6), GTPase, IMAP family member 7 (GIMAP7), C-X-C motif chemokine receptor 6 (CXCR6), dehydrogenase/reductase 9 (DHRS9), Fc gamma receptor Ic, pseudogene (FCGR1CP), and/or ankyrin repeat domain 34B (ANKRD34B) is elevated prior to ICI treatment (baseline), compared to the transcript levels in a control sample.

In some aspects, a subject is predicted as having a high risk of developing or diagnosed with having irAE if the transcript levels of one or more of LILRB4, CISH and/or PARP9 is elevated prior to ICI treatment (baseline), compared to a control sample. In some aspects, a subject is predicted as having a high risk of developing or diagnosed with having irAE if the transcript levels of one or more of LILRB4, CISH, GIMAP7, CXCR6, DHRS9, ANKRD34B, and/or FCGR1CP is elevated prior to ICI treatment (baseline), compared to the transcript levels in a control sample.

In some aspects, the subject is predicted as having a high risk of developing or diagnosed with having irAE if the transcript level of LILRB4 is elevated prior to ICI treatment, compared to the transcript level in a control sample. In some aspects, the subject is predicted as having a high risk of developing or diagnosed with having irAE if the transcript level of CISH is elevated prior to ICI treatment, compared to the transcript level in a control sample. In some aspects, the subject is predicted as having a high risk of developing or diagnosed with having irAE if the transcript level of PARP9 is elevated prior to ICI treatment, compared to the transcript level in a control sample. In some aspects, the subject is predicted as having a high risk of developing or diagnosed with having irAE if the transcript level RNF145 is elevated prior to ICI treatment, compared to the transcript level in a control sample. In some aspects, the subject is predicted as having a high risk of developing or diagnosed with having irAE if the transcript level ASGR2 is elevated prior to ICI treatment, compared to the transcript level in a control sample. In some aspects, the subject is predicted as having a high risk of developing or diagnosed with having irAE if the transcript level of SLC16A13 is elevated prior to ICI treatment, compared to the transcript level in a control sample. In some aspects, the subject is predicted as having a high risk of developing or diagnosed with having irAE if the transcript level of LPAR6 is elevated prior to ICI treatment, compared to the transcript level in a control sample. In some aspects, the subject is predicted as having a high risk of developing or diagnosed with having irAE if the transcript level of GIMAP7 is elevated prior to ICI treatment, compared to the transcript level in a control sample. In some aspects, the subject is predicted as having a high risk of developing or diagnosed with having irAE if the transcript level of CXCR6 is elevated prior to ICI treatment, compared to the transcript level in a control sample. In some aspects, the subject is predicted as having a high risk of developing or diagnosed with having irAE if the transcript level of DHRS9 is elevated prior to ICI treatment, compared to the transcript level in a control sample. In some aspects, the subject is predicted as having a high risk of developing or diagnosed with having irAE if the transcript level of FCGR1CP is elevated prior to ICI treatment, compared to the transcript level in a control sample. In some aspects, the subject is predicted as having a high risk of developing or diagnosed with having irAE if the transcript level of ANKRD34B is elevated prior to ICI treatment, compared to the transcript level in a control sample.

In some aspects, the subject is predicted as having a high risk of developing or diagnosed with having irAE if the transcript level of at least LILRB4 is elevated in the subject prior to ICI treatment compared to the transcript level in a control sample. In some aspects, the subject is predicted as having a high risk of developing or diagnosed with having irAE if the transcript level of at least CISH is elevated in the subject prior to ICI treatment compared to the transcript level in a control sample. In some aspects, the subject is predicted as having a high risk of developing or diagnosed with having irAE if the transcript level of at least PARP9 is elevated prior to ICI treatment, compared to the transcript level in a control sample. In some aspects, the subject is predicted as having a high risk of developing or diagnosed with having irAE if the transcript level of at least GIMAP7 is elevated in the subject prior to ICI treatment compared to the transcript level in a control sample. In some aspects, the subject is predicted as having a high risk of developing or diagnosed with having irAE if the transcript level of at least CXCR6 is elevated in the subject prior to ICI treatment compared to the transcript level in a control sample. In some aspects, the subject is predicted as having a high risk of developing or diagnosed with having irAE if the transcript level of at least DHRS9 is elevated prior to ICI treatment, compared to the transcript level in a control sample. In some aspects, the subject is predicted as having a high risk of developing or diagnosed with having irAE if the transcript level of at least FCGR1CP is elevated prior to ICI treatment, compared to the transcript level in a control sample. In some aspects, the subject is predicted as having a high risk of developing or diagnosed with having irAE if the transcript level of at least ANKRD34B is elevated prior to ICI treatment, compared to the transcript level in a control sample.

In some aspects, the subject is predicted as having a high risk of developing or diagnosed with having irAE if the transcript levels of one or more of Amphiregulin (AREG), epiregulin (EREG), Oncostatin M (OSM), cysteine and serine rich nuclear protein 1 (CSRNP1), DNA damage inducible transcript 4 (DDIT4), IL-10 (interleukin 10), Prostaglandin-endoperoxide synthase (PTGS2), Dual Specificity Phosphatase 1 (DUSP1), C-X-C chemokine receptor type 4 (CXCR4), Nuclear Factor, Interleukin 3 Regulated (NFIL3), Fos proto-oncogene, AP-1 transcription factor subunit (FOS), NFKB inhibitor alpha (NFKBIA), PPP1R15A (protein phosphatase 1 regulatory subunit 15A), CD79A, JunB proto-oncogene, AP-1 transcription factor subunit (JUNB), C-X-C motif chemokine ligand 8 (CXCL8), Early growth response 1 (EGR1), G0/G1 switch 2 (G0S2), paired box 8 (PAX8), activating transcription factor 6 beta (ATF6B), PAX8 antisense RNA1 (PAX8-AS1), RNA, variant U1 small nuclear 19 (RNVU1-19), vitelline membrane outer layer 1 homolog (VMO1), heparin binding EGF like growth factor (HBEGF), coiled-coil domain containing 144A (CCDC144A), shisa family member 8 (SHISA8), nuclear receptor subfamily 4 group A member 2 (NR4A2), prostaglandin E synthase (PTGES), synapsin I (SYN1), C-X-C motif chemokine ligand 2 (CXCL2), Peripheral myelin protein 22 (PMP22), CD83, early growth response 3 (EGR3), NUAK family kinase 1 (NUAK1), nocturnin (NOCT), atonal bHLH transcription factor 8 (ATOH8), polo like kinase (PLK2), inhibitor of DNA binding 1 (ID1), adrenoceptor beta 1 (ADRB1), snail family transcriptional repressor 1 (SNAIL), notch receptor 3 (NOTCH3), activating transcription factor 3 (ATF3), dual specificity phosphatase 2 (DUSP2), period circadian regulator 1 (PER1), TNF superfamily member 9 (TNFSF9), MAF bzip transcription factor F (MAFF), microRNA 4420 (MIR4420), glutathione peroxidase (GPX3), TNF alpha induced protein 3 (TNFAIP3), potassium voltage-gated channel modifier subfamily G member 1 (KCNG1), prostaglandin-endoperoxidase synthase 2 (PTGS2), A-kinase anchoring protein 5 (AKAP5), dual specificity phosphatase 1 (DUSP1), diacylglycerol kinase kappa (DGKK), beta-1,4,-N-acetyl-galactosaminyltransferase 3 (B4GALNT3), tribbles pseudokinase 1 (TRIB1), phorobol-12-myristate-13-acetate-induced protein 1 (PMAIP1), C-X-C motif chemokine receptor 4 (CXCR4), tumor protein p53 inducible nuclear protein 2 (TP53INP2), nuclear factor, interleukin 3 regulated (NFIL3), dual specificity phosphatase 4 (DUSP4), NFKB inhibitor alpha (NFKBIA), arginine vasopressin induced 1 (AVPI1), CD79a, ADP ribosylation factor like GTPase 4D (ARL4D), joining chain of multimeric IgA and IgM (JCHAIN), BTG anti-proliferation factor 2 (BTG2), TLE family member 1, transcriptional corepressor (TLE1), nuclear transport factor 2 like export factor 1 (NXT1), transducer of ERBB2, 1 (TOB1), phosphodiesterase 4D (PDE4D), DNAJ heat shock protein family member B1 (DNAJB1), AT-rich interaction domain 5B (ARID5B), G protein-coupled receptor 153 (GPR153), KLF transcription factor 9 (KLF9), SBDS ribosome maturation factor (SBDS), immediate early response 2 (IER2), TSC22 domain family member 3 (TSC22D3), GABA type A receptor associated protein like 1 (GABARAPL1), JunD proto-oncogene, AP-1 transcription factor subunit (JUND), RUNX family transcription factor 3 (RUNX3), BABAM2 antisense RNA 1 (BRE-AS1), putative salt inducible kinas 1B (LOC102724428), FAM46C (FAM46C), and/or general receptor for phosphoinositides 1-associated scaffold protein (GRASP) are lower in the subject when compared to the transcript levels in a control sample.

In some aspects, the subject is predicted as having a high risk of developing or diagnosed with having irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNA11, NOTCH3, ATF3, DUSP2, PER1, TNFSF9, MAFF, MIR4420, BRE-AS1, LOC102724428, and/or GRASP are lower in the subject when compared to the transcript levels in a control sample.

In some aspects, the subject is predicted as high risk of developing irAE or diagnosed with having irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A and/or JUNB are lower prior to ICI treatment, compared to the transcript levels in a control sample. In various aspects, the subject is predicted as high risk of developing irAE or diagnosed with having irAE if the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are lower prior to ICI treatment, compared to the transcript levels in a control sample. In some other aspects, the subject is predicted as high risk of developing irAE or diagnosed with having irAE if the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower prior to ICI treatment, compared to the transcript levels in a control sample.

In some aspects, the subject is predicted as having a high risk of developing or diagnosed with having irAE if the transcript level of at least AREG is lower in the subject when compared to the transcript level in a control sample. In some aspects, the subject is predicted as having a high risk of developing or diagnosed with having irAE if the transcript level of at least EREG is lower in the subject when compared to the transcript level in a control sample. In some aspects, the subject is predicted as having a high risk of developing or diagnosed with having irAE if the transcript level of at least OSM is lower in the subject when compared to the transcript level in a control sample. In some aspects, the subject is predicted as having a high risk of developing or diagnosed with having irAE if the transcript level of at least CSRNP1 is lower in the subject when compared to the transcript level in a control sample. In some aspects, the subject is predicted as having a high risk of developing or diagnosed with having irAE if the transcript level of at least DDIT4 is lower in the subject when compared to the transcript level in a control sample. In some aspects, the subject is predicted as having a high risk of developing or diagnosed with having irAE if the transcript level of at least IL-10 is lower in the subject when compared to the transcript level in a control sample. In some aspects, the subject is predicted as having a high risk of developing or diagnosed with having irAE if the transcript level of at least PTGS2 is lower in the subject when compared to the transcript level in a control sample. In some aspects, the subject is predicted as having a high risk of developing or diagnosed with having irAE if the transcript level of at least DUSP1 is lower in the subject when compared to the transcript level in a control sample. In some aspects, the subject is predicted as having a high risk of developing or diagnosed with having irAE if the transcript level of at least CXCR4 is lower in the subject when compared to the transcript level in a control sample. In some aspects, the subject is predicted as having a high risk of developing or diagnosed with having irAE if the transcript level of at least NFIL3 is lower in the subject when compared to the transcript level in a control sample. In some aspects, the subject is predicted as having a high risk of developing or diagnosed with having irAE if the transcript level of at least FOS is lower in the subject when compared to the transcript level in a control sample. In some aspects, the subject is predicted as having a high risk of developing or diagnosed with having irAE if the transcript level of at least NFKBIA is lower in the subject when compared to the transcript level in a control sample. In some aspects, the subject is predicted as having a high risk of developing or diagnosed with having irAE if the transcript level of at least PPP1R15A is lower in the subject when compared to the transcript level in a control sample. In some aspects, the subject is predicted as having a high risk of developing or diagnosed with having irAE if the transcript level of at least CD79A is lower in the subject when compared to the transcript level in a control sample. In some aspects, the subject is predicted as having a high risk of developing or diagnosed with having irAE if the transcript level of at least JUNB is lower in the subject when compared to the transcript level in a control sample. In some aspects, the subject is predicted as having a high risk of developing or diagnosed with having irAE if the transcript level of at least CXCL8 is lower in the subject when compared to the transcript level in a control sample. In some aspects, the subject is predicted as having a high risk of developing or diagnosed with having irAE if the transcript level of at least EGR1 is lower in the subject when compared to the transcript level in a control sample. In some aspects, the subject is predicted as having a high risk of developing or diagnosed with having irAE if the transcript level of at least G0S2 is lower in the subject when compared to the transcript level in a control sample. In some aspects, the subject is predicted as having a high risk of developing or diagnosed with having irAE if the transcript level of at least PAX8 is lower in the subject when compared to the transcript level in a control sample. In some aspects, the subject is predicted as having a high risk of developing or diagnosed with having irAE if the transcript level of at least ATF6B is lower in the subject when compared to the transcript level in a control sample. In some aspects, the subject is predicted as having a high risk of developing or diagnosed with having irAE if the transcript level of at least PAX8-AS1 is lower in the subject when compared to the transcript level in a control sample. In some aspects, the subject is predicted as having a high risk of developing or diagnosed with having irAE if the transcript level of at least RNVU1-19 is lower in the subject when compared to the transcript level in a control sample. In some aspects, the subject is predicted as having a high risk of developing or diagnosed with having irAE if the transcript level of at least VMO1 is lower in the subject when compared to the transcript level in a control sample. In some aspects, the subject is predicted as having a high risk of developing or diagnosed with having irAE if the transcript level of at least HBEGF is lower in the subject when compared to the transcript level in a control sample. In some aspects, the subject is predicted as having a high risk of developing or diagnosed with having irAE if the transcript level of at least CCDC144A is lower in the subject when compared to the transcript level in a control sample. In some aspects, the subject is predicted as having a high risk of developing or diagnosed with having irAE if the transcript level of at least SHISA8 is lower in the subject when compared to the transcript level in a control sample. In some aspects, the subject is predicted as having a high risk of developing or diagnosed with having irAE if the transcript level of at least NR4A2 is lower in the subject when compared to the transcript level in a control sample. In some aspects, the subject is predicted as having a high risk of developing or diagnosed with having irAE if the transcript level of at least PTGES is lower in the subject when compared to the transcript level in a control sample. In some aspects, the subject is predicted as having a high risk of developing or diagnosed with having irAE if the transcript level of at least SYN1 is lower in the subject when compared to the transcript level in a control sample. In some aspects, the subject is predicted as having a high risk of developing or diagnosed with having irAE if the transcript level of at least CXCL2 is lower in the subject when compared to the transcript level in a control sample. In some aspects, the subject is predicted as having a high risk of developing or diagnosed with having irAE if the transcript level of at least PMP22 is lower in the subject when compared to the transcript level in a control sample. In some aspects, the subject is predicted as having a high risk of developing or diagnosed with having irAE if the transcript level of at least CD83 is lower in the subject when compared to the transcript level in a control sample. In some aspects, the subject is predicted as having a high risk of developing or diagnosed with having irAE if the transcript level of at least EGR3 is lower in the subject when compared to the transcript level in a control sample. In some aspects, the subject is predicted as having a high risk of developing or diagnosed with having irAE if the transcript level of at least NUAK1 is lower in the subject when compared to the transcript level in a control sample. In some aspects, the subject is predicted as having a high risk of developing or diagnosed with having irAE if the transcript level of at least NOCT is lower in the subject when compared to the transcript level in a control sample. In some aspects, the subject is predicted as having a high risk of developing or diagnosed with having irAE if the transcript level of at least ATOH8 is lower in the subject when compared to the transcript level in a control sample. In some aspects, the subject is predicted as having a high risk of developing or diagnosed with having irAE if the transcript level of at least PLK2 is lower in the subject when compared to the transcript level in a control sample. In some aspects, the subject is predicted as having a high risk of developing or diagnosed with having irAE if the transcript level of at least ID1 is lower in the subject when compared to the transcript level in a control sample. In some aspects, the subject is predicted as having a high risk of developing or diagnosed with having irAE if the transcript level of at least ADRB1 is lower in the subject when compared to the transcript level in a control sample. In some aspects, the subject is predicted as having a high risk of developing or diagnosed with having irAE if the transcript level of at least SNAI1 is lower in the subject when compared to the transcript level in a control sample. In some aspects, the subject is predicted as having a high risk of developing or diagnosed with having irAE if the transcript level of at least NOTCH3 is lower in the subject when compared to the transcript level in a control sample. In some aspects, the subject is predicted as having a high risk of developing or diagnosed with having irAE if the transcript level of at least ATF3 is lower in the subject when compared to the transcript level in a control sample. In some aspects, the subject is predicted as having a high risk of developing or diagnosed with having irAE if the transcript level of DUSP2 is lower in the subject when compared to the transcript level in a control sample. In some aspects, the subject is predicted as having a high risk of developing or diagnosed with having irAE if the transcript level of at least PER1 is lower in the subject when compared to the transcript level in a control sample. In some aspects, the subject is predicted as having a high risk of developing or diagnosed with having irAE if the transcript level of at least TNFSF9 is lower in the subject when compared to the transcript level in a control sample. In some aspects, the subject is predicted as having a high risk of developing or diagnosed with having irAE if the transcript level of at least MAFF is lower in the subject when compared to the transcript level in a control sample. In some aspects, the subject is predicted as having a high risk of developing or diagnosed with having irAE if the transcript level of at least MI R4420 is lower in the subject when compared to the transcript level in a control sample. In some aspects, the subject is predicted as having a high risk of developing or diagnosed with having irAE if the transcript level of at least BRE-AS1 is lower in the subject when compared to the transcript level in a control sample. In some aspects, the subject is predicted as having a high risk of developing or diagnosed with having irAE if the transcript level of at least LOC102724428 is lower in the subject when compared to the transcript level in a control sample. In some aspects, the subject is predicted as having a high risk of developing or diagnosed with having irAE if the transcript level of at least GRASP is lower in the subject when compared to the transcript level in a control sample. In some aspects, the subject is predicted as having a high risk of developing or diagnosed with having irAE if the transcript level of at least TNFAIP3 is lower in the subject when compared to the transcript level in a control sample. In some aspects, the subject is predicted as having a high risk of developing or diagnosed with having irAE if the transcript level of at least TRIB1 is lower in the subject when compared to the transcript level in a control sample. In some aspects, the subject is predicted as having a high risk of developing or diagnosed with having irAE if the transcript level of at least PMAIP1 is lower in the subject when compared to the transcript level in a control sample. In some aspects, the subject is predicted as having a high risk of developing or diagnosed with having irAE if the transcript level of at least FAM46C is lower in the subject when compared to the transcript level in a control sample. In some aspects, the subject is predicted as having a high risk of developing or diagnosed with having irAE if the transcript level of at least NXT1 is lower in the subject when compared to the transcript level in a control sample. In some aspects, the subject is predicted as having a high risk of developing or diagnosed with having irAE if the transcript level of at least KLF9 is lower in the subject when compared to the transcript level in a control sample.

In some aspects, the subject is predicted as having a high risk of developing or diagnosed with having irAE if the transcript level of transcript of AREG is lower prior to ICI treatment, compared to the transcript level in a control sample. In some aspects, the subject is predicted as having a high risk of developing or diagnosed with having irAE if the transcript level of EREG is lower prior to ICI treatment, compared to the transcript level in a control sample. In some aspects, the subject is predicted as having a high risk of developing or diagnosed with having irAE if the transcript level of OSM is lower prior to ICI treatment, compared to the transcript level in a control sample. In some aspects, the subject is predicted as having a high risk of developing or diagnosed with having irAE if the transcript level of CSRNP1 is lower prior to ICI treatment, compared to the transcript level in a control sample. In some aspects, the subject is predicted as having a high risk of developing or diagnosed with having irAE if the transcript level of DDIT4 is lower prior to ICI treatment, compared to the transcript level in a control sample. In some aspects, the subject is predicted as having a high risk of developing or diagnosed with having irAE if the transcript level of IL-10 is lower prior to ICI treatment, compared to the transcript level in a control sample. In some aspects, the subject is predicted as having a high risk of developing or diagnosed with having irAE if the transcript level of PTGS2 is lower prior to ICI treatment, compared to the transcript level in a control sample. In some aspects, the subject is predicted as having a high risk of developing or diagnosed with having irAE if the transcript level of DUSP1 is lower prior to ICI treatment, compared to the transcript level in a control sample. In some aspects, the subject is predicted as having a high risk of developing or diagnosed with having irAE if the transcript level of CXCR4 is lower prior to ICI treatment, compared to the transcript level in a control sample. In some aspects, the subject is predicted as having a high risk of developing or diagnosed with having irAE if the transcript level of NIFL3 is lower prior to ICI treatment, compared to the transcript level in a control sample. In some aspects, the subject is predicted as having a high risk of developing or diagnosed with having irAE if the transcript level of FOS is lower prior to ICI treatment, compared to the transcript level in a control sample. In some aspects, the subject is predicted as having a high risk of developing or diagnosed with having irAE if the transcript level of NFKBIA is lower prior to ICI treatment, compared to the transcript level in a control sample. In some aspects, the subject is predicted as having a high risk of developing or diagnosed with having irAE if the transcript level of PPP1R15A is lower prior to ICI treatment, compared to the transcript level in a control sample. In some aspects, the subject is predicted as having a high risk of developing or diagnosed with having irAE if the transcript level of CD79A is lower prior to ICI treatment, compared to the transcript level in a control sample. In some aspects, the subject is predicted as having a high risk of developing or diagnosed with having irAE if the transcript level of JUNB is lower prior to ICI treatment, compared to the transcript level in a control sample.

In some aspects, the subject is predicted as having a high risk of developing or diagnosed with having irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are lower in the subject when compared to the level in the transcript levels in a control sample.

In some aspects, the subject is predicted as having a high risk of developing or diagnosed with having irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are lower in the subject when compared to the transcript levels in a control sample.

In some aspects, the subject is predicted as having a high risk of developing or diagnosed with having irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more transcript of AREG, EREG, OSM, CSRNP1, DDIT4, IL-10, PTGS2, DUSP1, CXCR4, NFIL3, FOS, NFKBIA, PPP1R15A, CD79A, and/or JUNB are lower in the subject when compared to the transcript levels in a control sample.

In some aspects, the subject is predicted as having a high risk of developing or diagnosed with having irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are lower in the subject when compared to the transcript levels in a control sample.

In some aspects, the subject is predicted as having a high risk of developing or diagnosed with having irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower in the subject when compared to the transcript levels in a control sample.

In some aspects, the subject is predicted as having a high risk of developing or diagnosed with having irAE if the transcript levels of one or more of LILRB4, CISH, and/or PARP9 are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are lower in the subject when compared to the transcript levels in a control sample.

In some aspects, the subject is predicted as having a high risk of developing or diagnosed with having irAE if the transcript levels of LILRB4, CISH, and PARP9 are elevated prior to ICI treatment (baseline), compared to the transcript levels in a control sample; and the transcript levels of AREG, EREG, OSM, CSRNP1, DDIT4, IL-10, PTGS2, DUSP1, CXCR4, NFIL3, FOS, NFKBIA, PPP1R15A, CD79A, and JUNB are lower in the subject when compared to the transcript levels in a control sample.

In some aspects, the subject is predicted as having a high risk of developing or diagnosed with having irAE if the transcript levels of one or more of LILRB4, GIMAP7, CISH, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are lower in the subject when compared to the transcript levels in a control sample.

In some aspects, the subject is predicted as having a high risk of developing or diagnosed with having irAE if the transcript level of CISH is elevated prior to ICI treatment, compared to the transcript level in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower in the subject when compared to the transcript levels in a control sample.

In some aspects, the subject is predicted as having a high risk of developing or diagnosed with having irAE if the transcript levels of one or more of LILRB4, GIMAP7, CISH, and/or CXCR6 are elevated prior to ICI treatment (baseline), compared to the transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 is lower in the subject when compared to the transcript levels in a control sample.

In some aspects, the subject is predicted as having a high risk of developing or diagnosed with having irAE if the transcript level of PARP9 is elevated prior to ICI treatment (baseline), compared to the transcript level in a control sample; and the transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are lower in the subject when compared to the transcript levels in a control sample.

In some aspects, the subject is predicted as having a high risk of developing or diagnosed with having irAE if the transcript levels of one or more of PARP9, CISH, CXCR6, LPAR6, and/or ASGR2 are elevated prior to ICI treatment (baseline), compared to the transcript levels in a control sample; and the transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNA11, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1 are lower in the subject when compared to the transcript levels in a control sample.

In some aspects, a subject is predicted as having a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to the transcript levels in a control sample.

In some aspects, a subject is predicted as having a low risk of developing irAE if the transcript levels of one or more of LILRB4, CISH and/or PARP9 is elevated prior to ICI treatment (baseline), compared to the transcript levels in a control sample. In some aspects, a subject is predicted as having a high risk of developing or diagnosed with having irAE if the transcript levels of one or more of LILRB4, CISH, GIMAP7, CXCR6, DHRS9, FCGR1CP, and/or ANKRD34B are lower prior to ICI treatment (baseline), compared to the transcript levels in a control sample.

In some aspects, the subject is predicted as having a low risk of developing irAE if the transcript level of LILRB4 is lower prior to ICI treatment, compared to the transcript level in a control sample. In some aspects, the subject is predicted as having a low risk of developing irAE if the transcript level of CISH is lower prior to ICI treatment, compared to the transcript level in a control sample. In some aspects, the subject is predicted as having a low risk of developing irAE if the transcript level of PARP9 is lower prior to ICI treatment, compared to the transcript level in a control sample. In some aspects, the subject is predicted as having a low risk of developing irAE if the transcript level of RNF145 is lower prior to ICI treatment, compared to the transcript level in a control sample. In some aspects, the subject is predicted as having a low risk of developing irAE if the transcript level of ASGR2 is lower prior to ICI treatment, compared to the transcript level in a control sample. In some aspects, the subject is predicted as having a low risk of developing irAE if the transcript level of SLC16A13 is lower prior to ICI treatment, compared to the transcript level in a control sample. In some aspects, the subject is predicted as having a low risk of developing irAE if the transcript level of LPAR6 is lower prior to ICI treatment, compared to the transcript level in a control sample. In some aspects, the subject is predicted as having a low risk of developing irAE if the transcript level of GIMAP7 is lower prior to ICI treatment, compared to the transcript level in a control sample. In some aspects, the subject is predicted as having a low risk of developing irAE if the transcript level of CXCR6 is lower prior to ICI treatment, compared to the transcript level in a control sample. In some aspects, the subject is predicted as having a low risk of developing irAE if the transcript level of DHRS9 is lower prior to ICI treatment, compared to the transcript level in a control sample. In some aspects, the subject is predicted as having a low risk of developing irAE if the transcript level of FCGR1CP is lower prior to ICI treatment, compared to the transcript level in a control sample. In some aspects, the subject is predicted as having a low risk of developing irAE if the transcript level of ANKRD34B is lower prior to ICI treatment, compared to the transcript level in a control sample.

In some aspects, the subject is predicted as having a low risk of developing irAE if the transcript level of at least LILRB4 is lower in the subject prior to ICI treatment compared to the transcript level in a control sample. In some aspects, the subject is predicted as having a low risk of developing irAE if the transcript level of at least CISH is lower in the subject prior to ICI treatment compared to the transcript level in a control sample. In some aspects, the subject is predicted as having a low risk of developing irAE if the transcript level of at least PARP9 is lower prior to ICI treatment, compared to the transcript level in a control sample. In some aspects, the subject is predicted as having low risk of developing irAE if the transcript level of at least GIMAP7 is lower in the subject prior to ICI treatment compared to the transcript level in a control sample. In some aspects, the subject is predicted as having low risk of developing irAE if the transcript level of at least CXCR6 is lower in the subject prior to ICI treatment compared to the transcript level in a control sample. In some aspects, the subject is predicted as having a low risk of developing irAE if the transcript level of at least DHRS9 is lower prior to ICI treatment, compared to the transcript level in a control sample. In some aspects, the subject is predicted as having a low risk of developing irAE if the transcript level of at least FCGR1CP is lower prior to ICI treatment, compared to the transcript level in a control sample. In some aspects, the subject is predicted as having a low risk of developing irAE if the transcript level of at least ANKRD34B is lower prior to ICI treatment, compared to the transcript level in control sample.

In some aspects, the subject is predicted or diagnosed as having a low risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are elevated in the subject when compared to the transcript levels in a control sample.

In some aspects, the subject is predicted or diagnosed as having a low risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP are elevated in the subject when compared to the transcript levels in a control sample.

In some aspects, the subject is predicted or diagnosed as a low risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A and/or JUNB are elevated prior to ICI treatment, compared to the transcript levels in a control sample. In various aspects, the subject is predicted or diagnosed as a low risk of developing irAE if the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are elevated prior to ICI treatment, compared to the transcript levels in a control sample. In some other aspects, the subject is predicted or diagnosed as a low risk of developing irAE if the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are elevated prior to ICI treatment, compared to the transcript levels in a control sample.

In some aspects, the subject is predicted or diagnosed as having a low risk of developing irAE if the transcript level of at least AREG is elevated in the subject when compared to the transcript level in a control sample. In some aspects, the subject is predicted or diagnosed as having a low risk of developing irAE if the transcript level of at least EREG is elevated in the subject when compared to the transcript level in a control sample. In some aspects, the subject is predicted or diagnosed as having a low risk of developing irAE if the transcript level of at least OSM is elevated in the subject when compared to the transcript level in a control sample. In some aspects, the subject is predicted or diagnosed as having a low risk of developing irAE if the transcript level of at least CSRNP1 is elevated in the subject when compared to the transcript level in a control sample. In some aspects, the subject is predicted or diagnosed as having a low risk of developing irAE if the transcript level of at least DDIT4 is elevated in the subject when compared to the transcript level in a control sample. In some aspects, the subject is predicted or diagnosed as having a low risk of developing irAE if the transcript level of at least IL-10 is elevated in the subject when compared to the level in the transcript level in a control sample. In some aspects, the subject is predicted or diagnosed as having a low risk of developing irAE if the transcript level of at least PTGS2 is elevated in the subject when compared to the transcript level in a control sample. In some aspects, the subject is predicted or diagnosed as having a low risk of developing irAE if the transcript level of at least DUSP1 is elevated in the subject when compared to the transcript level in a control sample. In some aspects, the subject is predicted or diagnosed as having a low risk of developing irAE if the transcript level of at least CXCR4 is elevated in the subject when compared to the transcript level in a control sample. In some aspects, the subject is predicted or diagnosed as having a low risk of developing irAE if the transcript level of at least NFIL3 is elevated in the subject when compared to the transcript level in a control sample. In some aspects, the subject is predicted or diagnosed as having a low risk of developing irAE if the transcript level of at least FOS is elevated in the subject when compared to transcript level in a control sample. In some aspects, the subject is predicted or diagnosed as having a low risk of developing irAE if the transcript level of at least NFKBIA is elevated in the subject when compared to the transcript level in a control sample. In some aspects, the subject is predicted or diagnosed as having a low risk of developing irAE if the transcript level of at least PPP1R15A is elevated in the subject when compared to the transcript level in a control sample. In some aspects, the subject is predicted or diagnosed as having a low risk of developing irAE if the transcript level of at least CD79A is elevated in the subject when compared to the transcript level in a control sample. In some aspects, the subject is predicted or diagnosed as having a low risk of developing irAE if the transcript level of at least JUNB is elevated in the subject when compared to the transcript level in a control sample. In some aspects, the subject is predicted or diagnosed as having a low risk of developing irAE if the transcript level of at least CXCL8 is elevated in the subject when compared to the level in a control sample. In some aspects, the subject is predicted or diagnosed as having a low risk of developing irAE if the transcript level of at least EGR1 is elevated in the subject when compared to the transcript level in a control sample. In some aspects, the subject is predicted or diagnosed as having a low risk of developing irAE if the transcript level of at least G0S2 is elevated in the subject when compared to the transcript level in a control sample. In some aspects, the subject is predicted or diagnosed as having a low risk of developing irAE if the transcript level of at least PAX8 is elevated in the subject when compared to the transcript level in a control sample. In some aspects, the subject is predicted or diagnosed as having a low risk of developing irAE if the transcript level of at least ATF6B is elevated in the subject when compared to the transcript level in a control sample. In some aspects, the subject is predicted or diagnosed as having a low risk of developing irAE if the transcript level of at least PAX8-AS1 is elevated in the subject when compared to the transcript level in a control sample. In some aspects, the subject is predicted or diagnosed as having a low risk of developing irAE if the transcript level of at least RNVU1-19 is elevated in the subject when compared to the transcript level in a control sample. In some aspects, the subject is predicted or diagnosed as having a low risk of developing irAE if the transcript level of at least VMO1 is elevated in the subject when compared to the transcript level in a control sample. In some aspects, the subject is predicted or diagnosed as having a low risk of developing irAE if the transcript level of at least HBEGF is elevated in the subject when compared to the transcript level in a control sample. In some aspects, the subject is predicted or diagnosed as having a low risk of developing irAE if the transcript level of at least CCDC144A is elevated in the subject when compared to the transcript level in a control sample. In some aspects, the subject is predicted or diagnosed as having a low risk of developing or diagnosed with having irAE if the transcript level of at least SHISA8 is elevated in the subject when compared to the transcript level in a control sample. In some aspects, the subject is predicted or diagnosed as having a low risk of developing or diagnosed with having irAE if the transcript level of at least NR4A2 is elevated in the subject when compared to the transcript level in a control sample. In some aspects, the subject is predicted or diagnosed as having a low risk of developing irAE if the transcript level of at least PTGES is elevated in the subject when compared to the transcript level in a control sample. In some aspects, the subject is predicted or diagnosed as having a low risk of developing irAE if the transcript level of at least SYN1 is elevated in the subject when compared to the transcript level in a control sample. In some aspects, the subject is predicted or diagnosed as having a low risk of developing irAE if the transcript level of at least CXCL2 is elevated in the subject when compared to the transcript level in a control sample. In some aspects, the subject is predicted or diagnosed as having a low risk of developing irAE if the transcript level of at least PMP22 is elevated in the subject when compared to the transcript level in a control sample. In some aspects, the subject is predicted or diagnosed as having a low risk of developing irAE if the transcript level of at least CD83 is elevated in the subject when compared to the transcript level in a control sample. In some aspects, the subject is predicted or diagnosed as having a low risk of developing irAE if the transcript level of at least EGR3 is elevated in the subject when compared to the transcript level in a control sample. In some aspects, the subject is predicted or diagnosed as having a low risk of developing irAE if the transcript level of at least NUAK1 is elevated in the subject when compared to the transcript level in a control sample. In some aspects, the subject is predicted or diagnosed as having a low risk of developing irAE if the transcript level of at least NOCT is elevated in the subject when compared to the transcript level in a control sample. In some aspects, the subject is predicted or diagnosed as having a low risk of developing irAE if the transcript level of at least ATOH8 is elevated in the subject when compared to the transcript level in a control sample. In some aspects, the subject is predicted or diagnosed as having a low risk of developing irAE if the transcript level of at least PLK2 is elevated in the subject when compared to the transcript levels in a control sample. In some aspects, the subject is predicted or diagnosed as having a low risk of developing irAE if the transcript level of at least ID1 is elevated in the subject when compared to the transcript level in a control sample. In some aspects, the subject is predicted or diagnosed as having a low risk of developing irAE if the transcript level of at least ADRB1 is elevated in the subject when compared to the transcript level in a control sample. In some aspects, the subject is predicted or diagnosed as having a low risk of developing irAE if the transcript level of at least SNAI1 is elevated in the subject when compared to the transcript level in a control sample. In some aspects, the subject is predicted or diagnosed as having a low risk of developing irAE if the transcript level of at least NOTCH3 is elevated in the subject when compared to the transcript level in a control sample. In some aspects, the subject is predicted or diagnosed as having a low risk of developing irAE if the transcript level of at least ATF3 is elevated in the subject when compared to the transcript level in a control sample. In some aspects, the subject is predicted or diagnosed as having a low risk of developing irAE if the transcript level of at least DUSP2 is elevated in the subject when compared to the transcript level in a control sample. In some aspects, the subject is predicted or diagnosed as having a low risk of developing irAE if the transcript level of at least PER1 is elevated in the subject when compared to the transcript level in a control sample. In some aspects, the subject is predicted or diagnosed as having a low risk of developing irAE if the transcript level of at least TNFSF9 is elevated in the subject when compared to the transcript level in a control sample. In some aspects, the subject is predicted or diagnosed as having a low risk of developing irAE if the transcript level of at least MAFF is elevated in the subject when compared to the transcript level in a control sample. In some aspects, the subject is predicted or diagnosed as having a low risk of developing irAE if the transcript level of at least MIR4420 is elevated in the subject when compared to the transcript level in a control sample. In some aspects, the subject is predicted or diagnosed as having a low risk of developing irAE if the transcript level of at least BRE-AS1 is elevated in the subject when compared to the transcript level in a control sample. In some aspects, the subject is predicted or diagnosed as having a low risk of developing irAE if the transcript level of at least LOC102724428 is elevated in the subject when compared to the transcript level in a control sample. In some aspects, the subject is predicted or diagnosed as having a low risk of developing irAE if the transcript level of at least GRASP is elevated in the subject when compared to the transcript level in a control sample. In some aspects, the subject is predicted or diagnosed as having a low risk of developing irAE if the transcript level of at least TNFAIP3 is elevated in the subject when compared to the transcript level in a control sample. In some aspects, the subject is predicted or diagnosed as having a low risk of developing irAE if the transcript level of at least TRIB1 is elevated in the subject when compared to the transcript level in a control sample. In some aspects, the subject is predicted or diagnosed as having a low risk of developing irAE if the transcript level of at least PMAIP1 is elevated in the subject when compared to the transcript level in a control sample. In some aspects, the subject is predicted or diagnosed as having a low risk of developing irAE if the transcript level of at least FAM46C is elevated in the subject when compared to the transcript level in a control sample. In some aspects, the subject is predicted or diagnosed as having a low risk of developing irAE if the transcript level of at least NXT1 is elevated in the subject when compared to the transcript level in a control sample. In some aspects, the subject is predicted or diagnosed as having a low risk of developing irAE if the transcript level of at least KLF9 is elevated in the subject when compared to the transcript level in a control sample.

In some aspects, the subject is predicted or diagnosed as having a low risk of developing irAE if the transcript level of AREG is elevated prior to ICI treatment, compared to the transcript level in a control sample. In some aspects, the subject is predicted or diagnosed as having a low risk of developing irAE if the transcript level of EREG is elevated prior to ICI treatment, compared to the transcript level in a control sample. In some aspects, the subject is predicted or diagnosed as having a low risk of developing irAE if the transcript level of OSM is elevated prior to ICI treatment, compared to the transcript level in a control sample. In some aspects, the subject is predicted or diagnosed as having a low risk of developing irAE if the transcript level of CSRNP1 is elevated prior to ICI treatment, compared to the transcript level in a control sample. In some aspects, the subject is predicted or diagnosed as having a low risk of developing irAE if the transcript level of DDIT4 is elevated prior to ICI treatment, compared to the transcript level in a control sample. In some aspects, the subject is predicted or diagnosed as having a low risk of developing irAE if the transcript level of IL-10 is elevated prior to ICI treatment, compared to the transcript level in a control sample. In some aspects, the subject is predicted or diagnosed as having a low risk of developing irAE if the transcript level of PTGS2 is elevated prior to ICI treatment, compared to the transcript level in a control sample. In some aspects, the subject is predicted or diagnosed as having a low risk of developing irAE if the transcript level of DUSP1 is elevated prior to ICI treatment, compared to transcript level in a control sample. In some aspects, the subject is predicted or diagnosed as having a low risk of developing irAE if the transcript level of CXCR4 is elevated prior to ICI treatment, compared to transcript level in a control sample. In some aspects, the subject is predicted or diagnosed as having a low risk of developing irAE if the transcript level of NIFL3 is elevated prior to ICI treatment, compared to transcript level in a control sample. In some aspects, the subject is predicted or diagnosed as having a low risk of developing irAE if the transcript level of FOS is elevated prior to ICI treatment, compared to transcript level in a control sample. In some aspects, the subject is predicted or diagnosed as having a low risk of developing irAE if the transcript level of NFKBIA is elevated prior to ICI treatment, compared to transcript level in a control sample. In some aspects, the subject is predicted or diagnosed as having a low risk of developing irAE if the transcript level of PPP1R15A is elevated prior to ICI treatment, compared to transcript level in a control sample. In some aspects, the subject is predicted or diagnosed as having a low risk of developing irAE if the transcript level of CD79A is elevated prior to ICI treatment, compared to transcript level in a control sample. In some aspects, the subject is predicted or diagnosed as having a low risk of developing irAE if the transcript level of JUNB is elevated prior to ICI treatment, compared to transcript level in a control sample.

In some aspects, the subject is predicted or diagnosed as having a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to transcript levels in a control sample; and the transcript levels transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP531NP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are elevated in the subject when compared to the transcript levels in a control sample.

In some aspects, the subject is predicted or diagnosed as having a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are elevated in the subject when compared to the transcript levels in a control sample.

In some aspects, the subject is predicted or diagnosed as having a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment, compared to transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are elevated in the subject when compared to the transcript levels in a control sample.

In some aspects, the subject is predicted or diagnosed as having a low risk of developing irAE if the transcript levels of one or more of LILRB4, CISH, PARP9, RNF145, ASGR2, SLC16A13, LPAR6, GIMAP7, and/or CXCR6 are lower prior to ICI treatment (baseline), compared to a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are elevated in the subject when compared to the transcript levels in a control sample.

In some aspects, the subject is predicted or diagnosed as having a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to transcript levels in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are elevated in the subject when compared to the transcript levels in a control sample.

In some aspects, the subject is predicted or diagnosed as having a low risk of developing irAE if the transcript levels of one or more of LILRB4, CISH, and/or PARP9 are lower prior to ICI treatment (baseline), compared to transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are elevated in the subject when compared to the transcript levels in a control sample.

In some aspects, the subject is predicted or diagnosed as having a low risk of developing irAE if the transcript levels of LILRB4, CISH, and PARP9 are lower prior to ICI treatment (baseline), compared to transcript levels in a control sample; and the transcript levels of AREG, EREG, OSM, CSRNP1, DDIT4, IL-10, PTGS2, DUSP1, CXCR4, NFIL3, FOS, NFKBIA, PPP1R15A, CD79A, and JUNB are elevated in the subject when compared to the transcript levels in a control sample.

In some aspects, the subject is predicted as having predicted or diagnosed as having a low risk of developing irAE if the transcript levels of one or more of LILRB4, GIMAP7, CISH, CXCR6, DHRS9, FCGR1CP, and/or ANKRD34B are lower prior to ICI treatment, compared to a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are elevated in the subject when compared to the transcript levels in a control sample.

In some aspects, the subject is predicted or diagnosed as having a low risk of developing irAE if the transcript level of CISH is lower prior to ICI treatment, compared to transcript level in a control sample; and the transcript level of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are elevated in the subject when compared to the transcript levels in a control sample.

In some aspects, the subject is predicted or diagnosed as having a low risk of developing irAE if the transcript levels of one or more of LILRB4, GIMAP7, CISH, CXCR6, DHRS9, FCGR1CP, and/or ANKRD34B are lower prior to ICI treatment (baseline), compared to transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are elevated in the subject when compared to the transcript levels in a control sample.

In some aspects, the subject is predicted or diagnosed as having a low risk of developing irAE if the transcript level of PARP9 is lower prior to ICI treatment (baseline), compared to transcript level in a control sample; and the transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are elevated in the subject when compared to the transcript levels in a control sample.

In some aspects, the subject is predicted or diagnosed as having a low risk of developing irAE if the transcript levels of one or more of PARP9, CISH, CXCR6, LPAR6, and/or ASGR2 are lower prior to ICI treatment (baseline), compared to transcript levels in a control sample; and the transcript levels of one or more transcript of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1 are elevated in the subject when compared to the transcript levels in a control sample.

In some aspects, the present disclosure provides a method of monitoring the risk of developing immune-related adverse events (irAE) associated with immune checkpoint inhibitor (ICI) treatment in a subject. The method comprises providing a sample from the subject, assessing transcript levels in the sample; and predicting the risk for developing/diagnosing irAE in the subject being monitored. In some aspects, assessment of transcripts comprises comparing the levels of one or more transcripts in the sample of the subject to the levels of the same transcripts in a control sample. In some aspects, transcript profile related to ICI-associated irAE comprises a transcript profile wherein the levels of one or more transcripts are elevated in the subject compared to the levels of the same transcripts in a control sample. In some aspects, transcript profile related to ICI-associated irAE comprises a transcript profile wherein the levels of one or more transcripts are decreased in the subject the levels of the same transcripts in a control sample.

In some aspects, the transcript is one or more of the transcripts disclosed in Table 5. In some aspects, the transcript is one or more of the transcripts disclosed in Table 6. In some aspects, the transcript is one or more of the transcripts disclosed in Table 8. In some aspects, the transcript is one or more of the transcripts disclosed in Table 9. In some aspects, the transcript is one or more of the transcripts disclosed in Table 10. In some aspects, the transcript is one or more of the transcripts disclosed in Table 11. In some aspects, transcript profile related to ICI-associated irAE comprises one or more transcripts with elevated level in the subject compared to the levels of the transcripts in a control sample and one or more transcripts with decreased level in the subject compared to the levels of the transcripts in a control sample. In some aspects, the transcript profile comprises one or more of the transcripts disclosed in Table 5, Table 6, Table 8, Table 9, Table 10, Table 11 or combinations thereof.

In some aspects, a subject being monitored can have a high risk of developing ICI-associated irAE, when the transcript levels of one or more transcripts are elevated in the subject compared to the transcript levels of the same transcripts in a control sample. In some aspects, a subject being monitored can have high risk of developing ICI-associated irAE, when the transcript levels of one or more transcripts are decreased in the subject compared to the transcript levels of the same transcripts in a control sample. In other aspects, a subject being monitored can have a low risk of developing ICI-associated irAE, when the transcript levels of one or more transcripts are elevated in the subject compared to the transcript levels of the same transcripts in a control sample. In another aspect, a subject being monitored can have allow risk of developing ICI-associated irAE, when the transcript levels of one or more transcripts are decreased in the subject compared to the transcript levels of the same transcripts in a control sample.

In some aspects, the transcript profile comprises a baseline or a pre-treatment transcript profile that correlates with future toxicity. These transcripts are used as biomarkers to assess the risk of a subject being monitored for developing irAE during ICI treatment. In some aspects, the subject is planning to undergo or is undergoing ICI treatment as part of a cancer therapy.

In some aspects, during monitoring, the transcript(s) having an elevated level has an elevated expression level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater as compared to sample.

In some aspects, during monitoring the transcript having elevated level which has an elevated expression level having a log 2 fold change value from about 0.1 to about 5. For example, a log 2 fold change value can be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5.

In some aspects, during monitoring the transcript(s) having decreased level has a decreased expression level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least 100% lower as compared to as compared to sample.

In some aspects, during monitoring the transcript having decreased level has decreased expression level having a log 2 fold change value from about −0.1 to about −5. For example, a log 2 fold change value can be about −0.1, −0.2, −0.3, −0.4, −0.5, −0.6, −0.7, −0.8, −0.9, −1, −1.1, −1.2, −1.3, −1.4, −1.5, −1.6, −1.7, −1.8, −1.9, −2, −2.1, −2.2, −2.3, −2.4, −2.5, −2.6, −2.7, −2.8, −2.9, −3, −3.1, −3.2, −3.3, −3.4, −3.5, −3.6, −3.7, −3.8, −3.9, −4, −4.1, −4.2, −4.3, −4.4, −4.5, −4.6, −4.7, −4.8, −4.9, or −5.

In some aspects, a subject being monitored can have a high risk of developing or diagnosed with having irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to transcript levels in a control sample.

In some aspects, a subject being monitored can have a high risk of developing or diagnosed with having irAE if the transcript levels of one or more of LILRB4, CISH and/or PARP9 are elevated prior to ICI treatment (baseline), compared to transcript levels in a control sample.

In some aspects, a subject being monitored can have a high risk of developing or diagnosed with having irAE if the transcript levels of one or more of LILRB4, CISH, GIMAP7, CXCR6, DHRS9, FCGR1CP, and/or ANKRD34B are elevated prior to ICI treatment (baseline), compared to transcript levels in a control sample.

In some aspects, the subject being monitored can have a high risk of developing irAE if the transcript level of LILRB4 is elevated prior to ICI treatment, compared to transcript level in a control sample. In some aspects, the subject being monitored can have a high risk of developing irAE if the transcript level of CISH is elevated prior to ICI treatment, compared to transcript level in a control sample. In some aspects, the subject being monitored can have a high risk of developing irAE if the transcript level of PARP9 is elevated prior to ICI treatment, compared to transcript level in a control sample. In some aspects, the subject being monitored can have a high risk of developing irAE if the transcript level of RNF145 is elevated prior to ICI treatment, compared to transcript level in a control sample. In some aspects, the subject being monitored can have a high risk of developing irAE if the transcript level of ASGR2 is elevated prior to ICI treatment, compared to transcript level in a control sample. In some aspects, the subject being monitored can have a high risk of developing irAE if the transcript level of SLC16A13 is elevated prior to ICI treatment, compared to transcript level in a control sample. In some aspects, the subject being monitored can have a high risk of developing irAE if the transcript level of LPAR6 is elevated prior to ICI treatment, compared to transcript level in a control sample. In some aspects, the subject being monitored can have a high risk of developing irAE if the transcript level of GIMAP7 is elevated prior to ICI treatment, compared to transcript level in a control sample. In some aspects, the subject being monitored can have a high risk of developing irAE if the transcript level of CXCR6 is elevated prior to ICI treatment, compared to transcript level a control sample. In some aspects, the subject being monitored can have a high risk of developing irAE if the transcript level of DHRS9 is elevated prior to ICI treatment, compared to transcript level in a control sample. In some aspects, the subject being monitored can have a high risk of developing irAE if the transcript level of FCGR1CP is elevated prior to ICI treatment, compared to transcript level in a control sample. In some aspects, the subject being monitored can have a high risk of developing irAE if the transcript level of ANKRD34B is elevated prior to ICI treatment, compared to transcript level in a control sample

In some aspects, the subject being monitored can have a high risk of developing irAE if the transcript level of at least LILRB4 is elevated in the subject prior to ICI treatment compared to transcript level in a control sample. In some aspects, the subject being monitored can have a high risk of developing irAE if the transcript level of at least CISH is elevated in the subject prior to ICI treatment compared to transcript level in a control sample. In some aspects, the subject being monitored can have a high risk of developing irAE if the transcript level of at least PARP9 is elevated prior to ICI treatment, compared to transcript level in a control sample. In some aspects, the subject being monitored can have a high risk of developing irAE if the transcript level of at least GIMAP7 is elevated in the subject prior to ICI treatment compared to transcript level in a control sample. In some aspects, the subject being monitored can have a high risk of developing irAE if the transcript level of at least CXCR6 is elevated in the subject prior to ICI treatment compared to transcript level in a control sample. In some aspects, the subject being monitored can have a high risk of developing irAE if the transcript level of at least DHRS9 is elevated prior to ICI treatment, compared to transcript level in a control sample. In some aspects, the subject being monitored can have a high risk of developing irAE if the transcript level of at least FCGR1CP is elevated prior to ICI treatment, compared to transcript level in a control sample. In some aspects, the subject being monitored can have a high risk of developing irAE if the transcript level of at least ANKRD34B is elevated prior to ICI treatment, compared to transcript level in a control sample

In some aspects, the subject being monitored can have a high risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, GABARAPL1, JUND, RUNX3, BRE-AS1, LOC102724428, FAM46C, and/or GRASP are lower in the subject when compared to the transcript levels in a control sample.

In some aspects, the subject being monitored can have a high risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP are lower in the subject when compared to the transcript levels in a control sample.

In some aspects, the subject being monitored can have a high risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are lower prior to ICI treatment, compared to transcript levels in a control sample. In various aspects, the subject being monitored can have a high risk of developing irAE if the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are lower prior to ICI treatment, compared to transcript levels in a control sample. In some other aspects, the subject being monitored can have a high risk of developing irAE if the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower prior to ICI treatment, compared to transcript levels in a control sample.

In some aspects, the subject being monitored can have a high risk of developing irAE if the transcript level of at least AREG is lower in the subject when compared to the transcript level in a control sample. In some aspects, the subject being monitored can have a high risk of developing irAE if the transcript level of at least EREG is lower in the subject when compared to the transcript level in a control sample. In some aspects, the subject being monitored can have a high risk of developing irAE if the transcript level of at least OSM is lower in the subject when compared to the transcript level in a control sample. In some aspects, the subject being monitored can have a high risk of developing irAE if the transcript level of at least CSRNP1 is lower in the subject when compared to the transcript level in a control sample. In some aspects, the subject being monitored can have a high risk of developing irAE if the transcript level of at least DDIT4 is lower in the subject when compared to the transcript level in a control sample. In some aspects, the subject being monitored can have a high risk of developing irAE if the transcript level of at least IL-10 is lower in the subject when compared to the transcript level in a control sample. In some aspects, the subject being monitored can have a high risk of developing irAE if the transcript level of at least PTGS2 is lower in the subject when compared to the transcript level in a control sample. In some aspects, the subject being monitored can have a high risk of developing irAE if the transcript level of at least DUSP1 is lower in the subject when compared to the transcript level in a control sample. In some aspects, the subject being monitored can have a high risk of developing irAE if the transcript level of at least CXCR4 is lower in the subject when compared to the transcript level in a control sample. In some aspects, the subject being monitored can have a high risk of developing irAE if the transcript level of at least NFIL3 is lower in the subject when compared to the transcript level in a control sample. In some aspects, the subject being monitored can have a high risk of developing irAE if the transcript level of at least FOS is lower in the subject when compared to the transcript level in a control sample. In some aspects, the subject being monitored can have a high risk of developing irAE if the transcript level of at least NFKBIA is lower in the subject when compared to the transcript level in a control sample. In some aspects, the subject being monitored can have a high risk of developing irAE if the transcript level of at least PPP1R15A is lower in the subject when compared to the transcript level in a control sample. In some aspects, the subject being monitored can have a high risk of developing irAE if the transcript level of at least CD79A is lower in the subject when compared to the transcript level in a control sample. In some aspects, the subject being monitored can have a high risk of developing irAE if the transcript level of at least JUNB is lower in the subject when compared to the transcript level in a control sample. In some aspects, the subject being monitored can have a high risk of developing irAE if the transcript level of at least CXCL8 is lower in the subject when compared to the transcript level in a control sample. In some aspects, the subject being monitored can have a high risk of developing irAE if the transcript level of at least EGR1 is lower in the subject when compared to the transcript level in a control sample. In some aspects, the subject being monitored can have a high risk of developing irAE if the transcript level of at least G0S2 is lower in the subject when compared to the transcript level in a control sample. In some aspects, the subject being monitored can have a high risk of developing irAE if the transcript level of at least PAX8 is lower in the subject when compared to the transcript level in a control sample. In some aspects, the subject being monitored can have a high risk of developing irAE if the transcript level of at least ATF6B is lower in the subject when compared to the transcript level in a control sample. In some aspects, the subject being monitored can have a high risk of developing irAE if the transcript level of at least PAX8-AS1 is lower in the subject when compared to the transcript level in a control sample. In some aspects, the subject being monitored can have a high risk of developing irAE if the transcript level of at least RNVU1-19 is lower in the subject when compared to the transcript level in a control sample. In some aspects, the subject being monitored can have a high risk of developing irAE if the transcript level of at least VMO1 is lower in the subject when compared to the transcript level in a control sample. In some aspects, the subject being monitored can have a high risk of developing irAE if the transcript level of at least HBEGF is lower in the subject when compared to the transcript level in a control sample. In some aspects, the subject being monitored can have a high risk of developing irAE if the transcript level of at least CCDC144A is lower in the subject when compared to the transcript level in a control sample. In some aspects, the subject being monitored can have a high risk of developing irAE if the transcript level of at least SHISA8 is lower in the subject when compared to the transcript level in a control sample. In some aspects, the subject being monitored can have a high risk of developing irAE if the transcript level of at least NR4A2 is lower in the subject when compared to the transcript level in a control sample. In some aspects, the subject being monitored can have a high risk of developing irAE if the transcript level of at least PTGES is lower in the subject when compared to the transcript level in a control sample. In some aspects, the subject being monitored can have a high risk of developing irAE if the transcript level of at least SYN1 is lower in the subject when compared to the transcript level in a control sample. In some aspects, the subject being monitored can have a high risk of developing irAE if the transcript level of at least CXCL2 is lower in the subject when compared to the transcript level in a control sample. In some aspects, the subject being monitored can have a high risk of developing irAE if the transcript level of at least PMP22 is lower in the subject when compared to the transcript level in a control sample. In some aspects, the subject being monitored can have a high risk of developing irAE if the transcript level of at least CD83 is lower in the subject when compared to the transcript level in a control sample. In some aspects, the subject being monitored can have a high risk of developing irAE if the transcript level of at least EGR3 is lower in the subject when compared to the transcript level in a control sample. In some aspects, the subject being monitored can have a high risk of developing irAE if the transcript level of at least NUAK1 is lower in the subject when compared to the transcript level in a control sample. In some aspects, the subject being monitored can have a high risk of developing irAE if the transcript level of at least NOCT is lower in the subject when compared to the transcript level in a control sample. In some aspects, the subject being monitored can have a high risk of developing irAE if the transcript level of at least ATOH8 is lower in the subject when compared to the transcript level in a control sample. In some aspects, the subject being monitored can have a high risk of developing irAE if the transcript level of at least PLK2 is lower in the subject when compared to the transcript level in a control sample. In some aspects, the subject being monitored can have a high risk of developing irAE if the transcript level of at least ID1 is lower in the subject when compared to the transcript level in a control sample. In some aspects, the subject being monitored can have a high risk of developing irAE if the transcript level of at least ADRB1 is lower in the subject when compared to the transcript level in a control sample. In some aspects, the subject being monitored can have a high risk of developing irAE if the transcript level of at least SNAI1 is lower in the subject when compared to the transcript level in a control sample. In some aspects, the subject being monitored can have a high risk of developing irAE if the transcript level of at least NOTCH3 is lower in the subject when compared to the transcript level in a control sample. In some aspects, the subject being monitored can have a high risk of developing irAE if the transcript level of at least ATF3 is lower in the subject when compared to the transcript level in a control sample. In some aspects, the subject being monitored can have a high risk of developing irAE if the transcript level of at least DUSP2 is lower in the subject when compared to the transcript level in a control sample. In some aspects, the subject being monitored can have a high risk of developing irAE if the transcript level of at least PER1 is lower in the subject when compared to the transcript level in a control sample. In some aspects, the subject being monitored can have a high risk of developing irAE if the transcript level of at least TNFSF9 is lower in the subject when compared to the transcript level in a control sample. In some aspects, the subject being monitored can have a high risk of developing irAE if the transcript level of at least MAFF is lower in the subject when compared to the transcript level in a control sample. In some aspects, the subject being monitored can have a high risk of developing irAE if the transcript level of at least MIR4420 is lower in the subject when compared to the transcript level in a control sample. In some aspects, the subject being monitored can have a high risk of developing irAE if the transcript level of at least BRE-AS1 is lower in the subject when compared to the transcript level in a control sample. In some aspects, the subject being monitored can have a high risk of developing irAE if the transcript level of at least LOC102724428 is lower in the subject when compared to the transcript level in a control sample. In some aspects, the subject being monitored can have a high risk of developing irAE if the transcript level of at least GRASP is lower in the subject when compared to the transcript level in a control sample. In some aspects, the subject being monitored can have a high risk of developing irAE if the transcript level of at least TNFAIP3 is lower in the subject when compared to the transcript level in a control sample. In some aspects, the subject being monitored can have a high risk of developing irAE if the transcript level of at least TRIB1 is lower in the subject when compared to the transcript level in a control sample. In some aspects, the subject being monitored can have a high risk of developing irAE if the transcript level of at least PMAIP1 is lower in the subject when compared to the transcript level in a control sample. In some aspects, the subject being monitored can have a high risk of developing irAE if the transcript level of at least FAM46C is lower in the subject when compared to the transcript level in a control sample. In some aspects, the subject being monitored can have a high risk of developing irAE if the transcript level of at least NXT1 is lower in the subject when compared to the transcript level in a control sample. In some aspects, the subject being monitored can have a high risk of developing irAE if the transcript level of at least KLF9 is lower in the subject when compared to the transcript level in a control sample.

In some aspects, the subject being monitored can have a high risk of developing irAE if the transcript level of AREG is lower prior to ICI treatment, compared to the transcript level in a control sample. In some aspects, the subject being monitored can have a high risk of developing irAE if the transcript level of EREG is lower prior to ICI treatment, compared to the transcript level in a control sample. In some aspects, the subject being monitored can have a high risk of developing irAE if the transcript level of OSM is lower prior to ICI treatment, compared to the transcript level in a control sample. In some aspects, the subject being monitored can have a high risk of developing irAE if the transcript level of CSRNP1 is lower prior to ICI treatment, compared to the transcript level in a control sample. In some aspects, the subject being monitored can have a high risk of developing irAE if the transcript level of DDIT4 is lower prior to ICI treatment, compared to the transcript level in a control sample. In some aspects, the subject being monitored can have a high risk of developing irAE if the transcript level of IL-10 is lower prior to ICI treatment, compared to the transcript level in a control sample. In some aspects, the subject being monitored can have a high risk of developing irAE if the transcript level of PTGS2 is lower prior to ICI treatment, compared to the transcript level in a control sample. In some aspects, the subject being monitored can have a high risk of developing irAE if the transcript level of DUSP1 is lower prior to ICI treatment, compared to the transcript level in a control sample. In some aspects, the subject being monitored can have a high risk of developing irAE if the transcript level of CXCR4 is lower prior to ICI treatment, compared to the transcript level a control sample. In some aspects, the subject being monitored can have a high risk of developing irAE if the transcript level of NIFL3 is lower prior to ICI treatment, compared to the transcript level in a control sample. In some aspects, the subject being monitored can have a high risk of developing irAE if the transcript level of FOS is lower prior to ICI treatment, compared to the transcript level in a control sample. In some aspects, the subject being monitored can have a high risk of developing irAE if the transcript level of NFKBIA is lower prior to ICI treatment, compared to the transcript level in a control sample. In some aspects, the subject being monitored can have a high risk of developing irAE if the transcript level of PPP1R15A is lower prior to ICI treatment, compared to the transcript level in a control sample. In some aspects, the subject being monitored can have a high risk of developing irAE if the transcript level of CD79A is lower prior to ICI treatment, compared to the transcript level in a control sample. In some aspects, the subject being monitored can have a high risk of developing irAE if the transcript level of JUNB is lower prior to ICI treatment, compared to the transcript in a control sample.

In some aspects, the subject being monitored can have a high risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are lower in the subject when compared to the transcript levels in a control sample.

In some aspects, the subject being monitored can have a high risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are lower in the subject when compared to the transcript levels in a control sample.

In some aspects, the subject being monitored can have a high risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are lower in the subject when compared to the transcript levels in a control sample.

In some aspects, the subject being monitored can have a high risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are lower in the subject when compared to the transcript levels in a control sample.

In some aspects, the subject being monitored can have a high risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower in the subject when compared to the transcript levels in a control sample.

In some aspects, the subject being monitored can have a high risk of developing irAE if the transcript levels of one or more of LILRB4, CISH, and/or PARP9 are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB is lower in the subject when compared to the transcript levels in a control sample.

In some aspects, the subject being monitored can have a high risk of developing irAE if the transcript levels of LILRB4, CISH, and PARP9 are elevated prior to ICI treatment (baseline), compared to a control sample; and the transcript levels of AREG, EREG, OSM, CSRNP1, DDIT4, IL-10, PTGS2, DUSP1, CXCR4, NFIL3, FOS, NFKBIA, PPP1R15A, CD79A, and JUNB are lower in the subject when compared to the transcript levels in a control sample.

In some aspects, the subject being monitored can have a high risk of developing irAE if the transcript levels of one or more of LILRB4, GIMAP7, CISH, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are lower in the subject when compared to the transcript levels in a control sample.

In some aspects, the subject being monitored can have a high risk of developing irAE if the transcript level of CISH is elevated prior to ICI treatment, compared to the transcript level in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower in the subject when compared to the transcript level in a control sample.

In some aspects, the subject being monitored can have a high risk of developing irAE if the transcript levels of one or more of LILRB4, GIMAP7, CISH, CXCR6, DHRS9, FCGR1CP, and/or ANKRD34B are elevated prior to ICI treatment (baseline), compared to the transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are lower in the subject when compared to the transcript levels in a control sample.

In some aspects, the subject being monitored can have a high risk of developing irAE if the transcript level of PARP9 is elevated prior to ICI treatment (baseline), compared to transcript levels in a control sample; and the transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are lower in the subject when compared to the transcript levels in a control sample.

In some aspects, the subject being monitored can have a high risk of developing irAE if the transcript levels of one or more of PARP9, CISH, CXCR6, LPAR6, and/or ASGR2 are elevated prior to ICI treatment (baseline), compared to the transcript levels in a control sample; and the transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1 are lower in the subject when compared to the transcript levels in a control sample.

In some aspects, the subject being monitored can have a low risk of developing irAE if the transcript levels of one or more transcript LILRB4, CISH, PARP9, RNF145, ASGR2, SLC16A13, LPAR6, GIMAP7), CXCR6 DHRS9, FCGR1CP, and/or ANKRD34B are lower prior to ICI treatment (baseline), compared to the transcript levels in a control sample.

In some aspects, the subject being monitored can have a low risk of developing irAE if the transcript levels of one or more of LILRB4, CISH and/or PARP9 are elevated prior to ICI treatment (baseline), compared to the transcript levels in a control sample. In some aspects, the subject being monitored can have a low risk of developing irAE if the transcript levels of one or more of LILRB4, CISH, GIMAP7, CXCR6, DHRS9, FCGR1CP, and/or ANKRD34B are lower prior to ICI treatment (baseline), compared to the transcript levels in a control sample.

In some aspects, the subject being monitored can have a low risk of developing irAE if the transcript level of LILRB4 is lower prior to ICI treatment, compared to the transcript level in a control sample. In some aspects, the subject being monitored can have a low risk of developing irAE if the transcript level of CISH is lower prior to ICI treatment, compared to the transcript level in a control sample. In some aspects, the subject being monitored can have a low risk of developing irAE if the transcript level of PARP9 is lower prior to ICI treatment, compared to the transcript level in a control sample. In some aspects, the subject being monitored can have a low risk of developing irAE if the transcript level of RNF145 is lower prior to ICI treatment, compared to the transcript level in a control sample. In some aspects, the subject being monitored can have a low risk of developing irAE if the transcript level of ASGR2 is lower prior to ICI treatment, compared to the transcript level in a control sample. In some aspects, the subject being monitored can have a low risk of developing irAE if the transcript level of SLC16A13 is lower prior to ICI treatment, compared to the transcript level in a control sample. In some aspects, the subject being monitored can have a low risk of developing irAE if the transcript level of LPAR6 is lower prior to ICI treatment, compared to the transcript level in a control sample. In some aspects, the subject being monitored can have a low risk of developing irAE if the transcript level of GIMAP7 is lower prior to ICI treatment, compared to the transcript level in a control sample. In some aspects, the subject being monitored can have a low risk of developing irAE if the transcript level of CXCR6 is lower prior to ICI treatment, compared to the transcript level in a control sample. In some aspects, the subject being monitored can have a low risk of developing irAE if the transcript level of DHRS9 is lower prior to ICI treatment, compared to the transcript level in a control sample. In some aspects, the subject being monitored can have a low risk of developing irAE if the transcript level of FCGR1CP is lower prior to ICI treatment, compared to the transcript level in a control sample. In some aspects, the subject being monitored can have a low risk of developing irAE if the transcript level of ANKRD34B is lower prior to ICI treatment, compared to the transcript level in a control sample.

In some aspects, the subject being monitored can have a low risk of developing irAE if the transcript level of at least LILRB4 is lower in the subject prior to ICI treatment compared to the transcript level in a control sample. In some aspects, the subject being monitored can have a low risk of developing irAE if the transcript level of at least CISH is lower in the subject prior to ICI treatment compared to the transcript level in a control sample. In some aspects, the subject being monitored can have a low risk of developing irAE if the transcript level of at least PARP9 is lower prior to ICI treatment, compared to the transcript level in a control sample. In some aspects, the subject being monitored can have a low risk of developing irAE if the transcript level of at least GIMAP7 is lower in the subject prior to ICI treatment compared to the transcript level in a control sample. In some aspects, the subject being monitored can have a low risk of developing irAE if the transcript level of at least CXCR6 is lower in the subject prior to ICI treatment compared to the transcript level in a control sample. In some aspects, the subject being monitored can have a low risk of developing irAE if the transcript level of at least DHRS9 is lower prior to ICI treatment, compared to the transcript level in a control sample. In some aspects, the subject being monitored can have a low risk of developing irAE if the transcript level of at least FCGR1CP is lower prior to ICI treatment, compared to the transcript level in a control sample. In some aspects, the subject being monitored can have a low risk of developing irAE if the transcript level of at least ANKRD34B is lower prior to ICI treatment, compared to the transcript level in a control sample.

In some aspects, the subject being monitored can have a low risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are elevated in the subject when compared to the transcript levels in a control sample.

In some aspects, the subject being monitored can have a low risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP are elevated in the subject when compared to the transcript levels in a control sample.

In some aspects, the subject being monitored can have a low risk of developing irAE if the transcript levels of one or more of AREG, EREG, OSM, CSRNP1, DDIT4, IL-10, PTGS2, DUSP1, CXCR4, NFIL3, FOS, NFKBIA, PPP1R15A, CD79A and/or JUNB is elevated prior to ICI treatment, compared to the transcript levels in a control sample. In various aspects, the subject being monitored can have a low risk of developing irAE if the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are elevated prior to ICI treatment, compared to the transcript levels in a control sample. In some other aspects, the subject being monitored can have a low risk of developing irAE if the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 is elevated prior to ICI treatment, compared to the transcript levels in a control sample.

In some aspects, the subject being monitored can have a low risk of developing irAE if the transcript level of at least AREG is elevated in the subject when compared to the transcript level in a control sample. In some aspects, the subject being monitored can have a low risk of developing irAE if the transcript level of at least EREG is elevated in the subject when compared to the transcript level in a control sample. In some aspects, the subject being monitored can have a low risk of developing irAE if the transcript level of at least OSM is elevated in the subject when compared to the transcript level in a control sample. In some aspects, the subject being monitored can have a low risk of developing irAE if the transcript level of at least CSRNP1 is elevated in the subject when compared to the transcript level in a control sample. In some aspects, the subject being monitored can have a low risk of developing irAE if the transcript level of at least DDIT4 is elevated in the subject when compared to the transcript level in a control sample. In some aspects, the subject being monitored can have a low risk of developing irAE if the transcript level of at least IL-10 is elevated in the subject when compared to the transcript level in a control sample. In some aspects, the subject being monitored can have a low risk of developing irAE if the transcript level of at least PTGS2 is elevated in the subject when compared to the transcript level in a control sample. In some aspects, the subject being monitored can have a low risk of developing irAE if the transcript level of at least DUSP1 is elevated in the subject when compared to the transcript level in a control sample. In some aspects, the subject being monitored can have a low risk of developing irAE if the transcript level of at least CXCR4 is elevated in the subject when compared to the transcript level in a control sample. In some aspects, the subject being monitored can have a low risk of developing irAE if the transcript level of at least NFIL3 is elevated in the subject when compared to the transcript level in a control sample. In some aspects, the subject being monitored can have a low risk of developing irAE if the transcript level of at least FOS is elevated in the subject when compared to the transcript level in a control sample. In some aspects, the subject being monitored can have a low risk of developing irAE if the transcript level of at least NFKBIA is elevated in the subject when compared to the transcript level in a control sample. In some aspects, the subject being monitored can have a low risk of developing irAE if the transcript level of at least PPP1R15A is elevated in the subject when compared to the transcript level in a control sample. In some aspects, the subject being monitored can have a low risk of developing irAE if the transcript level of at least CD79A is elevated in the subject when compared to the transcript level in a control sample. In some aspects, the subject being monitored can have a low risk of developing irAE if the transcript level of at least JUNB is elevated in the subject when compared to the transcript level in a control sample. In some aspects, the subject being monitored can have a low risk of developing irAE if the transcript level of at least CXCL8 is elevated in the subject when compared to the transcript level in a control sample. In some aspects, the subject being monitored can have a low risk of developing irAE if the transcript of at least EGR1 is elevated in the subject when compared to the transcript level in a control sample. In some aspects, the subject being monitored can have a low risk of developing irAE if the transcript level of at least G0S2 is elevated in the subject when compared to the transcript level in a control sample. In some aspects, the subject being monitored can have a low risk of developing irAE if the transcript level of at least PAX8 is elevated in the subject when compared to the transcript level in a control sample. In some aspects, the subject being monitored can have a low risk of developing irAE if the transcript level of at least ATF6B is elevated in the subject when compared to the transcript level in a control sample. In some aspects, the subject being monitored can have a low risk of developing irAE if the transcript level of at least PAX8-AS1 is elevated in the subject when compared to the transcript level in a control sample. In some aspects, the subject being monitored can have a low risk of developing irAE if the transcript level of at least RNVU1-19 is elevated in the subject when compared to the transcript level in a control sample. In some aspects, the subject being monitored can have a low risk of developing irAE if the transcript level of at least VMO1 is elevated in the subject when compared to the transcript level in a control sample. In some aspects, the subject being monitored can have a low risk of developing irAE if the transcript level of at least HBEGF is elevated in the subject when compared to the transcript level in a control sample. In some aspects, the subject being monitored can have a low risk of developing irAE if the transcript level of at least CCDC144A is lower in the subject when compared to the transcript level in a control sample. In some aspects, the subject being monitored can have a low risk of developing irAE if the transcript level of at least SHISA8 is lower in the subject when compared to the transcript level in a control sample. In some aspects, the subject being monitored can have a low risk of developing irAE if the transcript level of at least NR4A2 is lower in the subject when compared to the transcript level in a control sample. In some aspects, the subject being monitored can have a low risk of developing irAE if the transcript level of at least PTGES is elevated in the subject when compared to the transcript level in a control sample. In some aspects, the subject being monitored can have a low risk of developing irAE if the transcript level of at least SYN1 is elevated in the subject when compared to the transcript level in a control sample. In some aspects, the subject being monitored can have a low risk of developing irAE if the transcript level of at least CXCL2 is elevated in the subject when compared to the transcript level in a control sample. In some aspects, the subject being monitored can have a low risk of developing irAE if the transcript level of at least PMP22 is elevated in the subject when compared to the transcript level in a control sample. In some aspects, the subject being monitored can have a low risk of developing irAE if the transcript level of at least CD83 is elevated in the subject when compared to the transcript level in a control sample. In some aspects, the subject being monitored can have a low risk of developing irAE if the transcript level of at least EGR3 is elevated in the subject when compared to the transcript level in a control sample. In some aspects, the subject being monitored can have a low risk of developing irAE if the transcript level of at least NUAK1 is elevated in the subject when compared to the transcript level in a control sample. In some aspects, the subject being monitored can have a low risk of developing irAE if the transcript level of at least NOCT is elevated in the subject when compared to the transcript level in a control sample. In some aspects, the subject being monitored can have a low risk of developing irAE if the transcript level of at least ATOH8 is elevated in the subject when compared to the transcript level in a control sample. In some aspects, the subject being monitored can have a low risk of developing irAE if the transcript level of at least PLK2 is elevated in the subject when compared to the transcript level in a control sample. In some aspects, the subject being monitored can have a low risk of developing irAE if the transcript level of at least ID1 is elevated in the subject when compared to the transcript level in a control sample. In some aspects, the subject being monitored can have a low risk of developing irAE if the transcript level of at least ADRB1 is elevated in the subject when compared to the transcript level in a control sample. In some aspects, the subject being monitored can have a low risk of developing irAE if the transcript level of at least SNAI1 is elevated in the subject when compared to the transcript level in a control sample. In some aspects, the subject being monitored can have a low risk of developing irAE if the transcript level of at least NOTCH3 is elevated in the subject when compared to the transcript level in a control sample. In some aspects, the subject being monitored can have a low risk of developing irAE if the transcript level of at least ATF3 is elevated in the subject when compared to the transcript level in a control sample. In some aspects, the subject being monitored can have a low risk of developing irAE if the transcript level of at least DUSP2 is elevated in the subject when compared to the transcript level in a control sample. In some aspects, the subject being monitored can have a low risk of developing irAE if the transcript level of at least PER1 is elevated in the subject when compared to the transcript level in a control sample. In some aspects, the subject being monitored can have a low risk of developing irAE if the transcript level of at least TNFSF9 is elevated in the subject when compared to the transcript level in a control sample. In some aspects, the subject being monitored can have a low risk of developing irAE if the transcript level of at least MAFF is elevated in the subject when compared to the transcript level in a control sample. In some aspects, the subject being monitored can have a low risk of developing irAE if the transcript level of at least MI R4420 is elevated in the subject when compared to the transcript level in a control sample. In some aspects, the subject being monitored can have a low risk of developing irAE if the transcript level of at least BRE-AS1 is elevated in the subject when compared to the transcript level in a control sample. In some aspects, the subject being monitored can have a low risk of developing irAE if the transcript level of at least LOC102724428 is elevated in the subject when compared to the transcript level in a control sample. In some aspects, the subject being monitored can have a low risk of developing irAE if the transcript level of at least GRASP is elevated in the subject when compared to the transcript level in a control sample. In some aspects, the subject being monitored can have a low risk of developing irAE if the transcript level of at least TNFAIP3 is elevated in the subject when compared to the transcript level in a control sample. In some aspects, the subject being monitored can have a low risk of developing irAE if the transcript level of at least TRIB1 is elevated in the subject when compared to the transcript level in a control sample. In some aspects, the subject being monitored can have a low risk of developing irAE if the transcript level of at least PMAIP1 is elevated in the subject when compared to the transcript level in a control sample. In some aspects, the subject being monitored can have a low risk of developing irAE if the transcript level of at least FAM46C is elevated in the subject when compared to the transcript level in a control sample. In some aspects, the subject being monitored can have a low risk of developing irAE if the transcript level of at least NXT1 is elevated in the subject when compared to the transcript level in a control sample. In some aspects, the subject being monitored can have a low risk of developing irAE if the transcript level of at least KLF9 is elevated in the subject when compared to the transcript level in a control sample.

In some aspects, the subject being monitored can have a low risk of developing irAE if the transcript level of AREG is elevated prior to ICI treatment, compared to the transcript level in a control sample. In some aspects, the subject being monitored can have a low risk of developing irAE if the transcript level of EREG is elevated prior to ICI treatment, compared to the transcript level in a control sample. In some aspects, the subject being monitored can have a low risk of developing irAE if the transcript level of OSM is elevated prior to ICI treatment, compared to the transcript level in a control sample. In some aspects, the subject being monitored can have a low risk of developing irAE if the transcript level of CSRNP1 is elevated prior to ICI treatment, compared to the transcript level in a control sample. In some aspects, the subject being monitored can have a low risk of developing irAE if the transcript level of DDIT4 is elevated prior to ICI treatment, compared to the transcript level in a control sample. In some aspects, the subject being monitored can have a low risk of developing irAE if the transcript level of IL-10 is elevated prior to ICI treatment, compared to the transcript level in a control sample. In some aspects, the subject being monitored can have a low risk of developing irAE if the transcript level of PTGS2 is elevated prior to ICI treatment, compared to the transcript level in a control sample. In some aspects, the subject being monitored can have a low risk of developing irAE if the transcript level of DUSP1 is elevated prior to ICI treatment, compared to the transcript level in a control sample. In some aspects, the subject being monitored can have a low risk of developing irAE if the transcript level of CXCR4 is elevated prior to ICI treatment, compared to the transcript level in a control sample. In some aspects, the subject being monitored can have a low risk of developing irAE if the transcript level of NIFL3 is elevated prior to ICI treatment, compared to the transcript level in a control sample. In some aspects, the subject being monitored can have a low risk of developing irAE if the transcript level of FOS is elevated prior to ICI treatment, compared to the transcript level in a control sample. In some aspects, the subject being monitored can have a low risk of developing irAE if the transcript level of NFKBIA is elevated prior to ICI treatment, compared to the transcript level in a control sample. In some aspects, the subject being monitored can have a low risk of developing irAE if the transcript level of PPP1R15A is elevated prior to ICI treatment, compared to the transcript level in a control sample. In some aspects, the subject being monitored can have a low risk of developing irAE if the transcript level of CD79A is elevated prior to ICI treatment, compared to the transcript level in a control sample. In some aspects, the subject being monitored can have a low risk of developing irAE if the transcript level of JUNB is elevated prior to ICI treatment, compared to the transcript level in a control sample.

In some aspects, the subject being monitored can have a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are elevated in the subject when compared to the transcript levels in a control sample.

In some aspects, the subject being monitored can have a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are elevated in the subject when compared to the transcript levels in a control sample.

In some aspects, the subject being monitored can have a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment, compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are elevated in the subject when compared to the transcript levels in a control sample.

In some aspects, the subject being monitored can have a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are elevated in the subject when compared to the transcript levels in a control sample.

In some aspects, the subject being monitored can have a low risk of developing irAE if the transcript levels of one or more of LILRB4, CISH, PARP9, RNF145, ASGR2, SLC16A13, LPAR6, GIMAP7, CXCR6, DHRS9, FCGR1CP, and/or ANKRD34B is lower prior to ICI treatment (baseline), compared to the transcript levels in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are elevated in the subject when compared to the transcript levels in a control sample.

In some aspects, the subject being monitored can have a low risk of developing irAE if the transcript levels of one or more of LILRB4, CISH, and/or PARP9 are lower prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are elevated in the subject when compared to the transcript levels in a control sample.

In some aspects, the subject being monitored can have a low risk of developing irAE if the transcript levels of LILRB4, CISH, and PARP9 are lower prior to ICI treatment (baseline), compared to the transcript levels in a control sample; and the transcript levels of AREG, EREG, OSM, CSRNP1, DDIT4, IL-10, PTGS2, DUSP1, CXCR4, NFIL3, FOS, NFKBIA, PPP1R15A, CD79A, and JUNB are elevated in the subject when compared to the transcript levels in a control sample.

In some aspects, the subject being monitored can have a low risk of developing irAE if the transcript levels of one or more of LILRB4, GIMAP7, CISH, CXCR6, DHRS9, FCGR1CP, and/or ANKRD34B are lower prior to ICI treatment, compared to in the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are elevated in the subject when compared to the transcript levels in a control sample.

In some aspects, the subject being monitored can have a low risk of developing irAE if the transcript level of transcript CISH is lower prior to ICI treatment, compared to the transcript level a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are elevated in the subject when compared to the transcript levels in a control sample.

In some aspects, the subject being monitored can have a low risk of developing irAE if the transcript levels of one or more of LILRB4, GIMAP7, CISH, CXCR6, DHRS9, FCGR1CP, and/or ANKRD34B are lower prior to ICI treatment (baseline), compared to the transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are elevated in the subject when compared to the transcript levels in a control sample.

In some aspects, the subject being monitored can have a low risk of developing irAE if the transcript level of PARP9 is lower prior to ICI treatment (baseline), compared to transcript level in a control sample; and the transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are elevated in the subject when compared to the transcript levels in a control sample.

In some aspects, the subject being monitored can have a low risk of developing irAE if the transcript levels of one or more of PARP9, CISH, CXCR6, LPAR6, and/or ASGR2 are lower prior to ICI treatment (baseline), compared to the transcript levels in a control sample; and the transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1 are elevated in the subject when compared to the transcript levels in a control sample.

Any known method in the art can be used for measuring and/or monitoring the levels of transcripts. By way of non-limiting examples, levels of transcripts can be measured using RNA-seq, nanopore sequencing, Nanostring, multiplex RT-PCR, single-plex RT-PCR, NASBA, Fluorescence measurements or spectrophotometry. Samples to be tested can comprise whole blood, serum, plasma, urine, CSF or other suitable body fluid. Samples can be obtained from the subject before, during, and/or after ICI treatment, and quantification of the levels of transcripts can be performed, to assess the risk of irAE. In some aspects, transcription profile is a RNA peripheral blood transcription profile. The risk assessment comprises predicting, diagnosing, or monitoring ICI-related myositis, ICI-related myocarditis or ICI-related myositis and myocarditis.

### Autoantibody Profiles

In some aspects, the present disclosure provides autoantibody profile associated with ICI-related irAE. In some aspects, the autoantibody profile comprises autoantibody with an elevated expression in a subject with irAE at pre-treatment or baseline when compared to a control sample. In some aspects, the autoantibody profile comprises autoantibody with a lower expression in a subject with irAE at pre-treatment or baseline when compared to a control sample. In some aspects, the autoantibody is one or more of the autoantibody Mi-2, GAD65, Myosin, Thyroglobulin and/or TPO or any combinations thereof.

In some aspects, the autoantibody profile comprises a baseline or a pre-treatment transcript profile that correlates with future toxicity. These autoantibodies are used as biomarkers to assess the risk of a subject developing irAE during ICI treatment. In some aspects, the subject is planning to undergo or is undergoing ICI treatment as part of a cancer therapy.

In some aspects, the autoantibodies may comprise one or more of the autoantibodies disclosed in U.S. patent application Ser. No. 16/487,335 (U.S. Patent Application Publication No. US 2020/0284803), the disclosures of which are incorporated by reference in its entirety.

In some aspects, the present disclosure provides a method comprising predicting or diagnosing the subject as having a high risk of developing irAE if the expression of one or more autoantibody is elevated when compared to expression of the same autoantibody in a control sample. The method comprises providing a sample from the subject pretreatment or at baseline, assessing one or more autoantibodies in the sample, and predicting risk for developing or diagnosing as having irAE in the subject.

In some aspects, the method further comprising predicting or diagnosing the subject as having a high risk of developing irAE if the expression of one or more autoantibody is different in the subject when compared to the expression in control sample as described in U.S. patent application Ser. No. 16/487,335 (U.S. Patent Application Publication No. US 2020/0284803), the disclosures of which are incorporated by reference in its entirety.

In some aspects, the method of predicting or diagnosing a subject as having a high risk of developing irAE associated with ICI treatment comprises assessing the expression of one or more of the autoantibody Mi-2, GAD65, Myosin, Thyroglobulin and/or TPO or any combinations thereof. In some aspects, the subject is predicted or diagnosed as having a high risk of developing irAE if the expression of one or more autoantibody Mi-2, GAD65, Myosin, Thyroglobulin and/or TPO or any combinations thereof, is elevated when compared to the expression in a control sample. In some aspects, the subject is predicted or diagnosed as having a high risk of developing irAE if the expression of autoantibody Mi-2 is elevated when compared to the expression in a control sample. In some aspects, the subject is predicted or diagnosed as having a high risk of developing irAE if the expression of autoantibody GAD65 is elevated when compared to the expression in a control sample. In some aspects, the subject is predicted or diagnosed as having a high risk of developing irAE if the expression of autoantibody Myosin is elevated when compared to the expression in a control sample. In some aspects, the subject is predicted or diagnosed as having a high risk of developing irAE if the expression of autoantibody Thyroglobulin is elevated when compared to the expression in a control sample. In some aspects, the subject is predicted or diagnosed as having a high risk of developing irAE if the expression of autoantibody TPO is elevated when compared to the expression in a control sample.

In some aspects, the present disclosure provides a method comprising predicting or diagnosing the subject as having a low risk of developing irAE if the expression of one or more autoantibody is lower when compared to the expression in a control sample. The method comprises providing a sample from the subject pretreatment or at baseline, assessing one or more autoantibodies in the sample, and predicting risk for developing or diagnosing as having irAE in the subject.

In some aspects, the method further comprising predicting or diagnosing the subject as having a high risk of developing irAE if the expression of one or more autoantibody is different in the subject when compared to the expression in control sample as described in U.S. patent application Ser. No. 16/487,335 (U.S. Patent Application Publication No. US 2020/0284803), the disclosures of which are incorporated by reference in its entirety.

In some aspects, the subject is predicted or diagnosed as having a low risk of developing irAE if the expression the one or more of the autoantibody Mi-2, GAD65, Myosin, Thyroglobulin and/or TPO or any combinations thereof is lower compared to the expression in a control sample. In some aspects, the subject is predicted or diagnosed as having a low risk of developing irAE if the expression of autoantibody Mi-2 is lower when compared to the expression in a control sample. In some aspects, the subject is predicted or diagnosed as having a low risk of developing irAE if the expression of autoantibody GAD65 is lower when compared to the expression in a control sample. In some aspects, the subject is predicted or diagnosed as having a low risk of developing irAE if the expression of autoantibody Myosin is lower when compared to the expression in a control sample. In some aspects, the subject is predicted or diagnosed as having a low risk of developing irAE if the expression of autoantibody Thyroglobulin is lower when compared to the expression in a control sample. In some aspects, the subject is predicted or diagnosed as having a low risk of developing irAE if the expression of autoantibody TPO is lower when compared to the expression in a control sample.

In some aspects, during predicting or diagnosing the risk of irAE, one or more of the disclosed autoantibodies have an elevated expression of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater as compared to control sample. In some aspects, the disclosed autoantibodies can have an elevated expression greater than 100% expression as compared to the expression in a control sample.

In some aspects, during predicting or diagnosing the risk of irAE, one or more autoantibody can be expressed at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% lower as compared to control sample. In some aspects, one or more autoantibodies can be at least about 25% lower, at least 50% lower, or 100% lower, as compared to the expression in a control sample.

In some aspects, the disclosure provides an autoantibody profile for monitoring the risk of developing irAE associated with ICI treatment in a subject. In some aspects, the autoantibody profile comprises autoantibody with an elevated expression in a subject with irAE at pre-treatment or baseline when compared to the expression in a control sample. In some aspects, the autoantibody profile comprises autoantibody with a lower expression in a subject with irAE at pre-treatment or baseline when compared to the expression in a control sample. In some aspects, the autoantibody is one or more of the autoantibody Mi-2, GAD65, Myosin, Thyroglobulin and/or TPO or any combinations thereof.

In some aspects, the subject being monitored can have a high risk of developing irAE if the expression of one or more autoantibody is different in the subject when compared to the expression in control sample as described in U.S. patent application Ser. No. 16/487,335 (U.S. Patent Application Publication No. US 2020/0284803), the disclosures of which are incorporated by reference in its entirety.

In some aspects, the subject being monitored can have a high risk of developing irAE if the expression of one or more autoantibody Mi-2, GAD65, Myosin, Thyroglobulin and/or TPO or any combinations thereof, is elevated when compared to the expression in a control sample. In some aspects, the subject being monitored can have a high risk of developing irAE if the expression of autoantibody Mi-2 is elevated when compared to the expression in a control sample. In some aspects, the subject being monitored can have a high risk of developing irAE if the expression of autoantibody GAD65 is elevated when compared to the expression in a control sample. In some aspects, the subject being monitored can have a high risk of developing irAE if the expression of autoantibody Myosin is elevated when compared to the expression in a control sample. In some aspects, the subject being monitored can have a high risk of developing irAE if the expression of autoantibody Thyroglobulin is elevated when compared to the expression in a control sample. In some aspects, the subject being monitored can have a high risk of developing irAE if the expression of autoantibody TPO is elevated when compared to the expression in a control sample.

In some aspects, the subject being monitored can have a low risk of developing irAE if the expression of one or more autoantibody is different in the subject when compared to the expression in control sample as described in U.S. patent application Ser. No. 16/487,335 (U.S. Patent Application Publication No. US 2020/0284803), the disclosures of which are incorporated by reference in its entirety.

In some aspects, the subject being monitored can have a low risk of developing irAE if the expression of one or more autoantibody Mi-2, GAD65, Myosin, Thyroglobulin and/or TPO or any combinations thereof, is lower when compared to the expression in a control sample. In some aspects, the subject being monitored can have a low risk of developing irAE if the expression of autoantibody Mi-2 is lower when compared to the expression in a control sample.

In some aspects, the subject being monitored can have a low risk of developing irAE if the expression of autoantibody GAD65 is lower when compared to the expression in a control sample. In some aspects, the subject being monitored can have a low risk of developing irAE if the expression of autoantibody Myosin is lower when compared to the expression in a control sample. In some aspects, the subject being monitored can have a low risk of developing irAE if the expression of autoantibody Thyroglobulin is lower when compared to the expression in a control sample. In some aspects, the subject being monitored can have a low risk of developing irAE if the expression of autoantibody TPO is lower when compared to the expression in a control sample.

In some aspects, during monitoring the risk of irAE, one or more of the disclosed autoantibodies have an elevated expression of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater as compared to a control sample. In some aspects, the disclosed autoantibodies can have an elevated expression greater than 100% expression as compared to the expression in a control sample.

In some aspects, during monitoring the risk of irAE, one or more autoantibody can be expressed at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% lower as compared to a control sample. In some aspects, one or more autoantibodies can be at least about 25% lower, at least 50% lower, or 100% lower, as compared to the expression in a control sample.

Isolating, purifying, measuring and/or monitoring the expression of autoantibodies can be performed using any known method in the art. By way of non-limiting examples, autoantibody can be detected using enzyme linked immunosorbent assay (ELISA), radioimmunoassay (RIA), immunoradiometric assay, fluoroimmunoassay, chemiluminescent assay, bioluminescent assay, and Western blot. In some aspects, autoantibody profiling can be done using protein array panel comprising various antigens. By the way of non-limiting example, protein array panel can be a custom protein array panel of autoantigens, including nuclear antigens, cytosolic/matrix antigens, and tissue/organ-specific antigens. Samples to be tested may comprise whole blood, serum, plasma, urine, CSF, or other suitable body fluid. Samples can be obtained from the subject before, during and/or after ICI treatment, and quantification of expression of autoantibodies can be performed, to assess the risk of irAE. In some aspects, autoantibody profile is a blood plasma autoantibody profile. The risk assessment comprises predicting, diagnosing, or monitoring ICI-related myositis, ICI-related myocarditis or ICI-related myositis and myocarditis.

### Cytokine Profiles

In some aspects, the present disclosure provides cytokine profile associated with ICI-related irAE. In some aspects, the cytokine profile comprises one or more cytokine with an elevated level in a subject with irAE as compared to a control sample. In some aspects, the cytokine profile comprises one or more cytokine with a lower levels in a subject with irAE as compared to a control sample.

In some aspects, the cytokines are one or more of CXCL2, CXCL5, CXCL6, CCL7, CCL19, IFNγ, IL-6, CXCL9, CXCL10 or any combinations thereof. In some aspects, the disclosed cytokines may comprise one or more of the cytokines disclosed in U.S. patent application Ser. No. 14/045,482 (U.S. Patent Application Publication No. US 2021/0263045), the disclosures of which are incorporated by reference in its entirety.

In some aspects, the cytokine profile comprises a baseline or a pre-treatment cytokine that correlates with future toxicity. In some aspects, the cytokine profile comprises a cytokine with sustained expression during treatment. In some aspects, these cytokines are used as biomarkers to assess the risk of a subject developing irAE before, during or after ICI treatment.

In some aspects, the method of predicting or diagnosing a subject as having a high risk of developing irAE associated with ICI treatment comprises assessing the expression of one or more of the cytokine CXCL2, CXCL5, CXCL6, CCL7, CCL19, IFNγ, IL-6, CXCL9, CXCL10 or any combinations thereof. In some aspects, the subject is predicted or diagnosed as having a high risk of developing irAE if the expression of one or more cytokine CXCL2, CXCL5, CXCL6, CCL7, CCL19, IFNγ, IL-6, CXCL9, CXCL10 or any combinations thereof, is elevated when compared to the expression in a control sample. In some aspects, the subject is predicted or diagnosed as having a high risk of developing irAE if the expression of cytokine CXCL2 is elevated when compared to the expression in a control sample. In some aspects, the subject is predicted or diagnosed as having a high risk of developing irAE if the expression of cytokine CXCL5 is elevated when compared to the expression in a control sample. In some aspects, the subject is predicted or diagnosed as having a high risk of developing irAE if the expression of cytokine CXCL6 is elevated when compared to the expression in a control sample. In some aspects, the subject is predicted or diagnosed as having a high risk of developing irAE if the expression of cytokine CCL7 is elevated when compared to the expression in a control sample. In some aspects, the subject is predicted or diagnosed as having a high risk of developing irAE if the expression of cytokine CCL19 is elevated when compared to the expression in a control sample. In some aspects, the subject is predicted or diagnosed as having a high risk of developing irAE if the expression of cytokine IFNγ is elevated when compared to the expression in a control sample. In some aspects, the subject is predicted or diagnosed as having a high risk of developing irAE if the expression of cytokine IL-6 is elevated when compared to the expression in a control sample. In some aspects, the subject is predicted or diagnosed as having a high risk of developing irAE if the expression of cytokine CXCL9 is elevated when compared to the expression in a control sample. In some aspects, the subject is predicted or diagnosed as having a high risk of developing irAE if the expression of cytokine CXCL10 is elevated when compared to the expression in a control sample.

In some aspects, the cytokine profile comprises a cytokine with sustained elevated expression after initiation of ICI treatment. In some aspects, the cytokine with sustained elevated expression after initiation of ICI treatment is one or more of CXCL5, IL-6, IFN-γ, CXCL9, CXCL10 or any combinations thereof. In some aspects, the cytokine with sustained elevated expression is CXCL5. In some aspects, the cytokine with sustained elevated expression is IL-6. In some aspects, the cytokine with sustained elevated expression is IFN-γ. In some aspects, the cytokine with sustained elevated expression is CXCL9. In some aspects, the cytokine with sustained elevated expression is CXCL10.

In some aspects, the method of predicting or diagnosing a subject as having a low risk of developing irAE associated with ICI treatment comprises assessing the expression of one or more of the cytokine CXCL2, CXCL5, CXCL6, CCL7, CCL19, IFNγ, IL-6, CXCL9, CXCL10 or any combinations thereof. In some aspects, the subject is predicted or diagnosed as having a low risk of developing irAE if the expression of one or more cytokine CXCL2, CXCL5, CXCL6, CCL7, CCL19, IFNγ, IL-6, CXCL9, CXCL10 or any combinations thereof, is lower when compared to the expression in a control sample. In some aspects, the subject is predicted or diagnosed as having a low risk of developing irAE if the expression of cytokine CXCL2 is lower when compared to a control sample. In some aspects, the subject is predicted or diagnosed as having a low risk of developing irAE if the expression of cytokine CXCL5 is lower when compared to the expression in a control sample. In some aspects, the subject is predicted or diagnosed as having a low risk of developing irAE if the expression of cytokine CXCL6 is lower when compared to the expression in a control sample. In some aspects, the subject is predicted or diagnosed as having a low risk of developing irAE if the expression of cytokine CCL7 is lower when compared to the expression in a control sample. In some aspects, the subject is predicted or diagnosed as having a low risk of developing irAE if the expression of cytokine CCL19 is lower when compared to the expression in a control sample. In some aspects, the subject is predicted or diagnosed as having a low risk of developing irAE if the expression of cytokine IFNγ is lower when compared to the expression in a control sample. In some aspects, the subject is predicted or diagnosed as having a low risk of developing irAE if the expression of cytokine IL-6 is lower when compared to the expression in a control sample. In some aspects, the subject is predicted or diagnosed as having a low risk of developing irAE if the expression of cytokine CXCL9 is lower when compared to the expression in a control sample. In some aspects, the subject is predicted or diagnosed as having a low risk of developing irAE if the expression of cytokine CXCL10 is lower when compared to the expression in a control sample.

In some aspects, during prediction or diagnosis, one or more of the disclosed cytokine having an elevated expression has an elevated expression level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater when compared to the expression in a control sample.

In some aspects, during prediction or diagnosis, one or more of the disclosed cytokine having a lower expression has an expression level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% lower when compared to the expression in a control sample.

In some aspects, during prediction or diagnosis, one or more of the disclosed cytokines has a concentration of about 10 pg/ml, about 20 pg/ml, about 30 pg/ml, about 40 pg/ml, about 50 pg/ml, about 60 pg/ml, about 70 pg/ml, about 80 pg/ml, about 90 pg/ml, about 100 pg/ml, about 120 pg/ml, about 140 pg/ml, about 150 pg/ml, about 160 pg/ml, about 180 pg/ml, about 200 pg/ml, about 300 pg/ml, about 400 pg/ml, about 500 pg/ml, about 600 pg/ml, about 700 pg/ml, or about 800 pg/ml.

In some aspects, during prediction or diagnosis, one or more of the disclosed cytokine has an elevated expression, wherein the expression has a fold change of at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 when compared to the expression in a control sample.

In some aspects, the disclosure provides a cytokine profile for monitoring the risk of developing irAE associated with ICI treatment in a subject. In some aspects, the cytokine profile comprises one or more cytokine with an elevated level in a subject with irAE as compared to a control sample. In some aspects, the cytokine profile comprises one or more cytokine with an lower levels in a subject with irAE as compared to the expression in a control sample.

In some aspects, the cytokines for monitoring the risk of developing irAE associated with ICI treatment are one or more of CXCL2, CXCL5, CXCL6, CCL7, CCL19, IFNγ, IL-6, CXCL9, CXCL10 or any combinations thereof. In some aspects, the disclosed cytokines may comprise one or more of the cytokines disclosed in U.S. patent application Ser. No. 14/045,482 (U.S. Patent Application Publication No. US 2021/0263045), the disclosures of which are incorporated by reference in its entirety.

In some aspects, the cytokine profile for monitoring the risk of developing irAE associated with ICI treatment comprises a baseline or a pre-treatment cytokine that correlates with future toxicity. In some aspects, the cytokine profile for monitoring the risk of developing irAE associated with ICI treatment comprises a cytokine with sustained expression during treatment. In some aspects, these cytokines are used as biomarkers to assess the risk of a subject developing irAE before, during or after ICI treatment.

In some aspects, the subject being monitored can have a high risk of developing irAE if the expression of one or more cytokine CXCL2, CXCL5, CXCL6, CCL7, CCL19, IFNγ, IL-6, CXCL9, CXCL10 or any combinations thereof, is elevated when compared to the expression in a control sample. In some aspects, the subject being monitored can have a high risk of developing irAE if the expression of cytokine CXCL2 is elevated when compared to the expression in a control sample. In some aspects, the subject being monitored can have a high risk of developing irAE if the expression of cytokine CXCL5 is elevated when compared to the expression in a control sample. In some aspects, the subject being monitored can have a high risk of developing irAE if the expression of cytokine CXCL6 is elevated when compared to the expression in a control sample. In some aspects, the subject being monitored can have a high risk of developing irAE if the expression of cytokine CCL7 is elevated when compared to the expression in a control sample. In some aspects, the subject being monitored can have a high risk of developing irAE if the expression of cytokine CCL19 is elevated when compared to the expression in a control sample. In some aspects, the subject being monitored can have a high risk of developing irAE if the expression of cytokine IFNγ is elevated when compared to the expression in a control sample. In some aspects, the subject being monitored can have a high risk of developing irAE if the expression of cytokine IL-6 is elevated when compared to the expression in a control sample. In some aspects, the subject being monitored can have a high risk of developing irAE if the expression of cytokine CXCL9 is elevated when compared to the expression in a control sample. In some aspects, the subject being monitored can have a high risk of developing irAE if the expression of cytokine CXCL10 is elevated when compared to the expression in a control sample.

In some aspects, the cytokine profile for monitoring the risk of developing irAE associated with ICI treatment comprises a cytokine with sustained elevated expression after initiation of ICI treatment. In some aspects, the cytokine with sustained elevated expression after initiation of ICI treatment is one or more of CXCL5, IL-6, IFN-γ, CXCL9, CXCL10 or any combinations thereof. In some aspects, the cytokine with sustained elevated expression is CXCL5. In some aspects, the cytokine with sustained elevated expression is IL-6. In some aspects, the cytokine with sustained elevated expression is IFN-γ. In some aspects, the cytokine with sustained elevated expression is CXCL9. In some aspects, the cytokine with sustained elevated expression is CXCL10.

In some aspects, the subject being monitored can have a low risk of developing irAE if the expression of one or more of the cytokine CXCL2, CXCL5, CXCL6, CCL7, CCL19, IFNγ, IL-6, CXCL9, CXCL10 or any combinations thereof is lower when compared to the expression in a control sample. In some aspects, the subject being monitored can have a high risk of developing irAE if the expression of cytokine CXCL2 is lower when compared to the expression in a control sample. In some aspects, the subject being monitored can have a high risk of developing irAE if the expression of cytokine CXCL5 is lower when compared to the expression in a control sample. In some aspects, the subject being monitored can have a high risk of developing irAE if the expression of cytokine CXCL6 is lower when compared to the expression in a control sample. In some aspects, the subject being monitored can have a high risk of developing irAE if the expression of cytokine CCL7 is lower when compared to the expression in a control sample. In some aspects, the subject being monitored can have a high risk of developing irAE if the expression of cytokine CCL19 is lower when compared to the expression in a control sample. In some aspects, the subject being monitored can have a high risk of developing irAE if the expression of cytokine IFNγ is lower when compared to the expression in a control sample. In some aspects, the subject being monitored can have a high risk of developing irAE if the expression of cytokine IL-6 is lower when compared to the expression in a control sample. In some aspects, the subject being monitored can have a high risk of developing irAE if the expression of cytokine CXCL9 is lower when compared to the expression in a control sample. In some aspects, the subject being monitored can have a high risk of developing irAE if the expression of cytokine CXCL10 is lower when compared to the expression in a control sample.

In some aspects, during monitoring one or more of the disclosed cytokine having an elevated expression has an elevated expression level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater when compared to the expression in a control sample.

In some aspects, during monitoring one or more of the disclosed cytokine having a lower expression has an expression level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% lower when compared to the expression in a control sample.

In some aspects, during monitoring, one or more of the disclosed cytokine has a concentration of about 10 pg/ml, about 20 pg/ml, about 30 pg/ml, about 40 pg/ml, about 50 pg/ml, about 60 pg/ml, about 70 pg/ml, about 80 pg/ml, about 90 pg/ml, about 100 pg/ml, about 120 pg/ml, about 140 pg/ml, about 150 pg/ml, about 160 pg/ml, about 180 pg/ml, about 200 pg/ml, about 300 pg/ml, about 400 pg/ml, about 500 pg/ml, about 600 pg/ml, about 700 pg/ml, or about 800 pg/ml.

In some aspects, during monitoring one or more of the disclosed cytokine has an elevated expression, wherein the expression has a fold change of at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 when compared to the expression in a control sample.

Detection, quantification and/or monitoring of cytokines can be conducted using well known methods in the art including enzyme linked immunosorbent assay (ELISA), radioimmunoassay (RIA), immunoradiometric assay, fluoroimmunoassay, chemiluminescent assay, bioluminescent assay, and Western blot. In some aspects, monitoring of cytokine levels is performed using readily available cytokine panels (e.g. Bio-Plex Pro Human Chemokine 40-plex Panel, Bio-Rad Laboratories, Hercules, California). Concentrations of cytokines can be determined on the basis of the fit of a provided standard curve for mean fluorescence intensity. Samples to be tested may comprise whole blood, serum, plasma, urine, CSF or other suitable body fluid. Samples can be obtained from the subject before, during and/or after ICI treatment, and quantification of cytokine concentration can be performed, to assess the risk of irAE. In some aspects, the samples are obtained from the subject before the initiation of ICI treatment. The risk assessment comprises predicting, diagnosing, or monitoring ICI-related myositis, ICI-related myocarditis or ICI-related myositis and myocarditis.

### Immune Cell Profiles

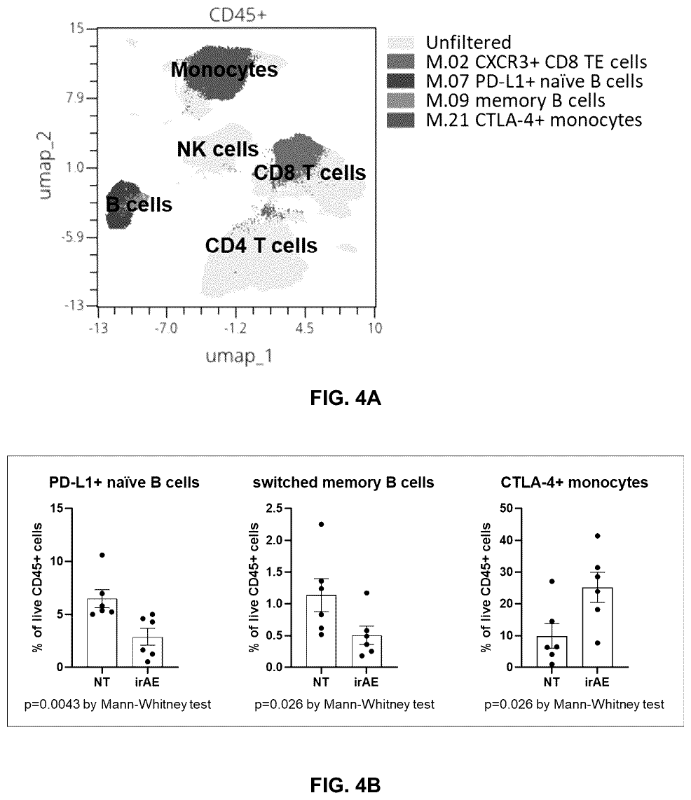

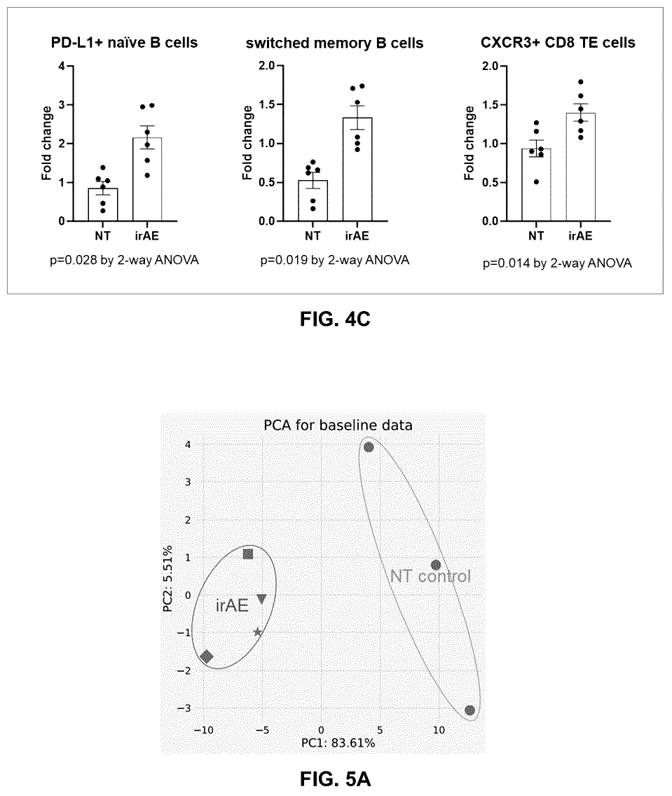

In some aspects, the present disclosure provides immune cell profile related to ICI-associated irAE. In some aspects, the immune cell profile comprises presence of a different immune cell subset in a subject with irAE when compared to immune cell subset in a control sample. In some aspects, the immune cell profile comprises one or more of the immune cell disclosed in FIG. 4A or any combinations thereof.

In some aspects, the immune cell profile comprises a baseline or a pre-treatment immune cell subset that correlates with future toxicity. In some aspects, the immune cell subsets are used as biomarkers to assess the risk of a subject developing irAE before or during ICI treatment. In some aspects, the immune cell profile comprises one or more an immune cell subsets with elevated abundance at pretreatment or baseline samples from the subject when compared to abundance of the same immune cell subset in a control sample. In some aspects, the immune cell profile comprises one or more an immune cell subsets with lower abundance at pretreatment or baseline samples from the subject when compared to abundance of the same immune cell subset in a control sample. In some aspects, the immune cell profile comprises an immune cell subset with increased change in abundance after initiation of ICI treatment in the sample from the subject when compared to abundance of the same immune cell subset in a control sample. In some aspects, the immune cell subsets comprise PD-L1+ naïve B cell, switched memory B cell, CTLA4+ monocyte, and CXCR3+ CD8 T cell.

In some aspects, the disclosure provides a method of predicting or diagnosing a subject as having a high risk of developing irAE associated with ICI treatment comprising assessing the abundance of one or more of the immune cell subsets PD-L1+ naïve B cell, switched memory B cell, and/or CTLA4+ monocyte. In some aspects, the subject is predicted or diagnosed as having a high risk of developing irAE if the abundance of one or more immune cell subset PD-L1+ naïve B cell, switched memory B cell or any combinations thereof, is lower and/or if the abundance of CTLA4+ monocyte is elevated when compared to the abundance in a control sample. In some aspects, the subject is predicted or diagnosed as having a high risk of developing irAE if the abundance of immune cell subset PD-L1+ naïve B cell is lower when compared to a control sample. In some aspects, the subject is predicted or diagnosed as having a high risk of developing irAE if the abundance of immune cell subset switched memory B cell is lower when compared to the abundance in a control sample. In some aspects, the subject is predicted or diagnosed as having a high risk of developing irAE if the abundance of immune cell subset CTLA4+ monocyte is elevated when compared to the abundance in a control sample.

In some aspects, the disclosure provides a method of predicting or diagnosing a subject as having a low risk of developing irAE associated with ICI treatment comprising assessing the abundance of one or more of the immune cell subsets PD-L1+ naïve B cell, switched memory B cell, and/or CTLA4+ monocyte. In some aspects, the subject is predicted or diagnosed as having a low risk of developing irAE if the abundance of one or more immune cell subset PD-L1+ naïve B cell, switched memory B cell or any combinations thereof, is elevated and/or if the abundance of CTLA4+ monocyte is lower when compared to the abundance in a control sample. In some aspects, the subject is predicted or diagnosed as having a low risk of developing irAE if the abundance of immune cell subset PD-L1+ naïve B cell is elevated when compared to the abundance in a control sample. In some aspects, the subject is predicted or diagnosed as having a low risk of developing irAE if the abundance of immune cell subset switched memory B cell is elevated when compared to the abundance in a control sample. In some aspects, the subject is predicted or diagnosed as having a low risk of developing irAE if the abundance of immune cell subset CTLA4+ monocyte is lower when compared to the abundance in a control sample.

In some aspects, the subject is predicted or diagnosed as having a high risk of developing irAE if the change in abundance of one or more immune cell subset PD-L1+ naïve B cell, switched memory B cell, and/or CXCR3+ CD8 T cell is enhanced after initiation of ICI treatment in the sample from the subject, when compared to the abundance in a control sample. In some aspects, the subject is predicted or diagnosed as having a high risk of developing irAE if the change in the abundance of immune cell subset PD-L1+ naïve B cell is enhanced after initiation of ICI treatment when compared to the abundance in a control sample. In some aspects, the subject is predicted or diagnosed as having a high risk of developing irAE if the change in the abundance of immune cell subset switched memory B cell is enhanced after initiation of ICI treatment when compared to the abundance in a control sample. In some aspects, the subject is predicted or diagnosed as having a high risk of developing irAE if the change in the abundance of immune cell subset CXCR3+ CD8 T cell is enhanced after initiation of ICI treatment when compared to the abundance in a control sample.

In some aspects, during prediction or diagnosis, the abundance of immune cells in an immune cell subset can be determined from a sample by determining the percentage of immune cells in the sample that fall into the subset. (For example, X % of the immune cells in the sample are subset 1.) The percentage of immune cells in a given subset can then be compared to the percentage of immune cells in that subset in a control sample. In some aspects, the abundance (e.g., percentage) of immune cells in the one or more immune cell subset can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points lower as compared to the abundance of the immune cells in the one or more immune cell subset in a control sample.

In some aspects, during prediction or diagnosis, the abundance of immune cells in an immune cell subset can be determined from a sample by determining the percentage of immune cells in the sample that fall into the subset. (For example, X % of the immune cells in the sample are subset 1.) The percentage of immune cells in a given subset can then be compared to the percentage of immune cells in that subset in control sample. In some aspects, the abundance (e.g., percentage) of immune cells in the one or more immune cell subset can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points greater as compared to the abundance of the immune cells in the one or more immune cell subset in a control sample.

In some aspects, a method of the disclosure provides an immune cell profile for monitoring the risk of developing irAE associated with ICI treatment in a subject. In some aspects, the immune cell profile comprises presence of a different immune cell subset in a subject with irAE when compared to a control sample. In some aspects, the immune cell profile comprises one or more of the immune cell disclosed in FIG. 4A or any combinations thereof.

In some aspects, the method of monitoring comprises providing a sample from the subject before ICI treatment, assessing the immune cell profile in the subject and comparing the immune profile to a control sample. In some aspect, an immune cell profile comprises a baseline or a pre-treatment immune cell subset that correlates with future toxicity. In some aspects, the immune cell subsets are used as biomarkers to assess the risk of a subject developing irAE before or during ICI treatment. In some aspects, the immune cell profile comprises one or more an immune cell subsets with elevated abundance at pretreatment or baseline samples from the subject when compared to the abundance in a control sample. In some aspects, the immune cell profile comprises one or more an immune cell subsets with lower abundance at pretreatment or baseline samples from the subject when compared to the abundance in a control sample. In some aspects, the immune cell profile comprises an immune cell subset with increased change in abundance after initiation of ICI treatment in the sample from the subject when compared to a control sample. In some aspects, the immune cell subsets comprise PD-L1+ naïve B cell, switched memory B cell, CTLA4+ monocyte, and CXCR3+ CD8 T cell.

In some aspects, the subject being monitored has a high risk of developing irAE associated with ICI treatment if the abundance of one or more immune cell subset PD-L1+ naïve B cell, switched memory B cell or any combinations thereof, is lower and/or if the abundance of CTLA4+ monocyte is elevated when compared to the abundance in a control sample. In some aspects the subject being monitored has a high risk of developing irAE if the abundance of immune cell subset PD-L1+ naïve B cell is lower when compared to a control sample. In some aspects, the subject being monitored has a high risk of developing irAE if the abundance of immune cell subset switched memory B cell is lower when compared to the abundance in a control sample. In some aspects, the subject being monitored has a high risk of developing irAE if the abundance of immune cell subset CTLA4+ monocyte is elevated when compared to the abundance in a control sample.

In some aspects, the subject being monitored has a low risk of developing irAE if the abundance of one or more immune cell subset PD-L1+ naïve B cell, switched memory B cell or any combinations thereof, is elevated and/or if the abundance of CTLA4+ monocyte is lower when compared to the abundance in a control sample. In some aspects, the subject being monitored has a low risk of developing irAE if the abundance of immune cell subset PD-L1+ naïve B cell is elevated when compared to the abundance in a control sample. In some aspects, the subject being monitored has a low risk of developing irAE if the abundance of immune cell subset switched memory B cell is elevated when compared to the abundance in a control sample. In some aspects, the subject being monitored has a low risk of developing irAE if the abundance of immune cell subset CTLA4+ monocyte is lower when compared to the abundance in a control sample.

In some aspects, the subject being monitored has a high risk of developing irAE if the change in abundance of one or more immune cell subset PD-L1+ naïve B cell, switched memory B cell, and/or CXCR3+ CD8 T cell is enhanced after initiation of ICI treatment in the sample from the subject, when compared to the abundance in a control sample. In some aspects, the subject being monitored has a high risk of developing irAE if the change in the abundance of immune cell subset PD-L1+ naïve B cell is enhanced after initiation of ICI treatment when compared to the abundance in a control sample. In some aspects, the subject being monitored has a high risk of developing irAE if the change in the abundance of immune cell subset switched memory B cell is enhanced after initiation of ICI treatment when compared to the abundance in a control sample. In some aspects, the subject being monitored has a high risk of developing irAE if the change in the abundance of immune cell subset CXCR3+ CD8 T cell is enhanced after initiation of ICI treatment when compared to the abundance in a control sample.

In some aspects, during monitoring, the abundance of immune cells in an immune cell subset can be determined from a sample by determining the percentage of immune cells in the sample that fall into the subset. (For example, X % of the immune cells in the sample are subset 1.) The percentage of immune cells in a given subset can then be compared to the percentage of immune cells in that subset in control sample. In some aspects, the abundance (e.g., percentage) of immune cells in the one or more immune cell subset can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points lower as compared to the abundance of the immune cells in the one or more immune cell subset in a control sample.

In some aspects, during monitoring, the abundance of immune cells in an immune cell subset can be determined from a sample by determining the percentage of immune cells in the sample that fall into the subset. (For example, X % of the immune cells in the sample are subset 1.) The percentage of immune cells in a given subset can then be compared to the percentage of immune cells in that subset in a control sample. In some aspects, the abundance (e.g., percentage) of immune cells in the one or more immune cell subset can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points greater as compared to the abundance of the immune cells in the one or more immune cell subset in a control sample.

Immune cell signatures can be detected, quantified and/or monitored using well known methods in the art including immune profiling assay, mass cytometry (cytometry by time-of-flight CyTOF), flow cytometry and cell sorting including FACS and immunomagnetic separation. In some aspects, immune cell signature is detected using high-dimensional mass cytometry (cytometry by time-of-flight (CyTOF)). Samples to be tested may comprise whole blood, serum, plasma, urine, CSF or other suitable body fluid. Samples can be obtained from the subject before, during and/or after ICI treatment, and immune cell signatures is identified, to assess the risk of irAE. The risk assessment comprises predicting, diagnosing, or monitoring ICI-related myositis, ICI-related myocarditis or ICI-related myositis and myocarditis.

In some aspects, the disclosed methods to assess the risk of a subject developing irAE is assessing the risk of ICI-associated myositis, ICI-associated myocarditis, or ICI-associated combined myositis and myocarditis. Myositis and myocarditis may occur as isolated toxicity or may occur along with one or more other ICI associated irAE including ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, and/or neutropenia. The autoimmune inflammatory myopathies (e.g., myositis or myocarditis) could develop spontaneously or as paraneoplastic phenomena.

### Combination Profiles

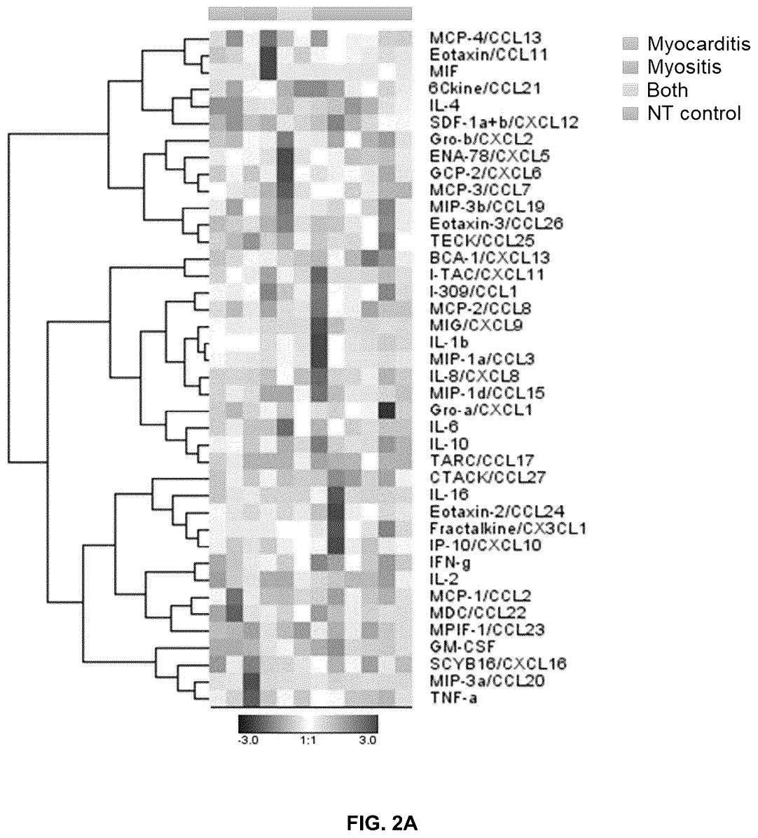

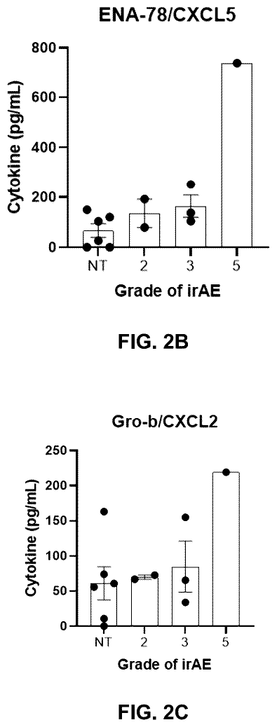

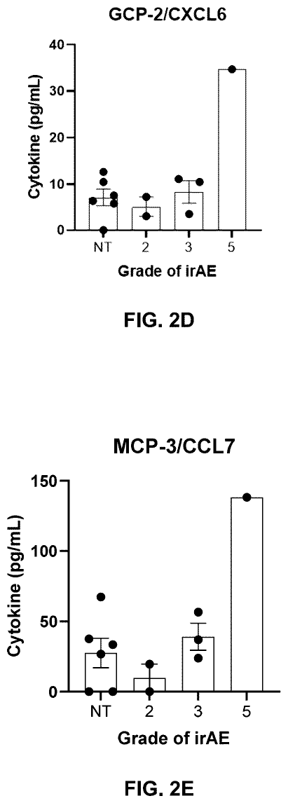

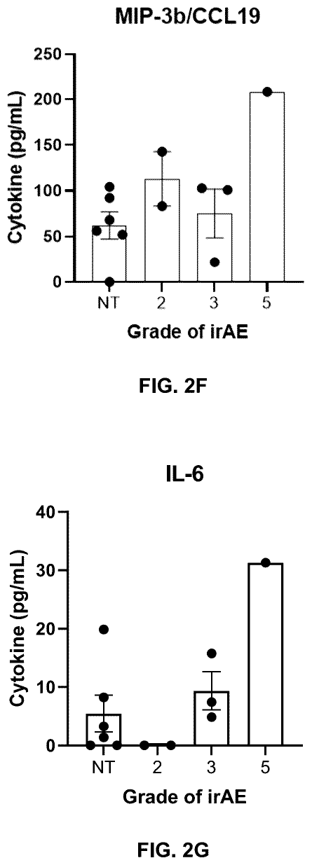

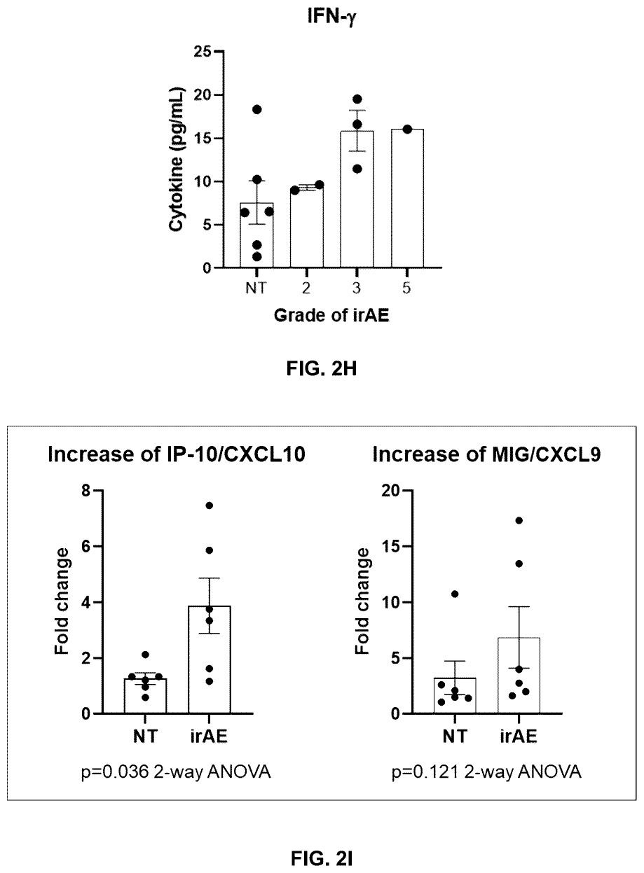

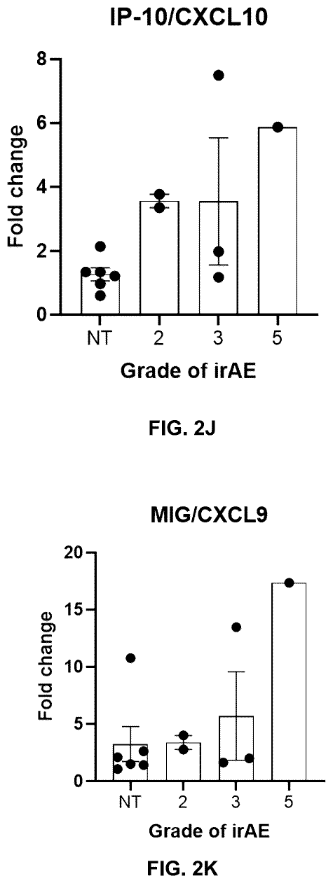

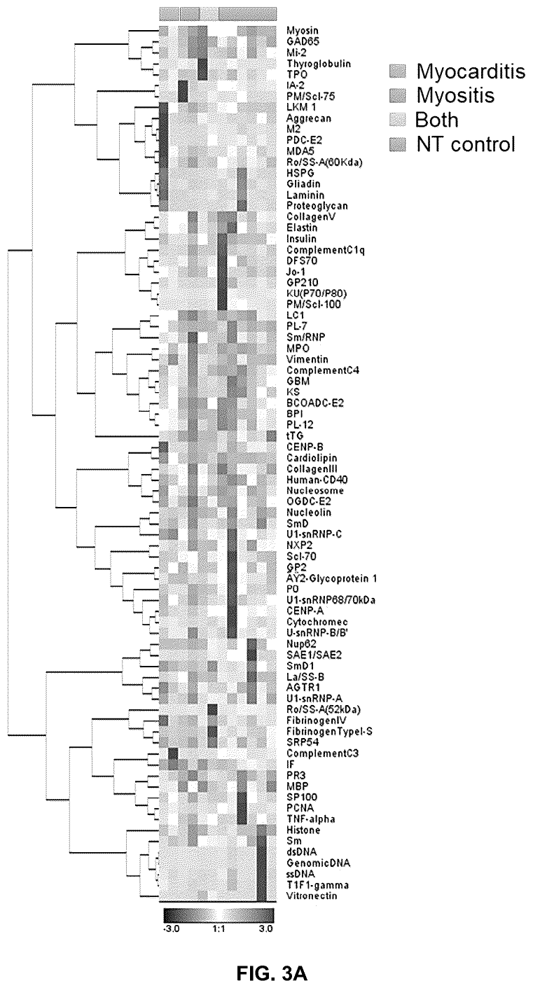

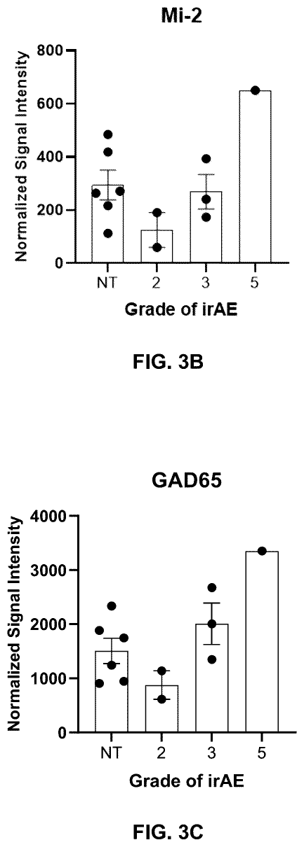

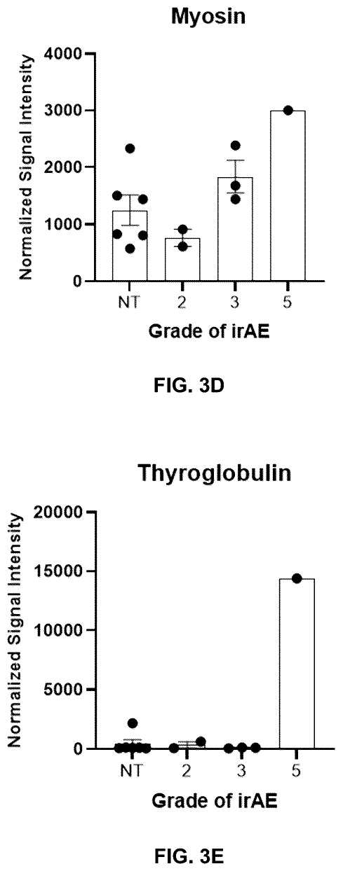

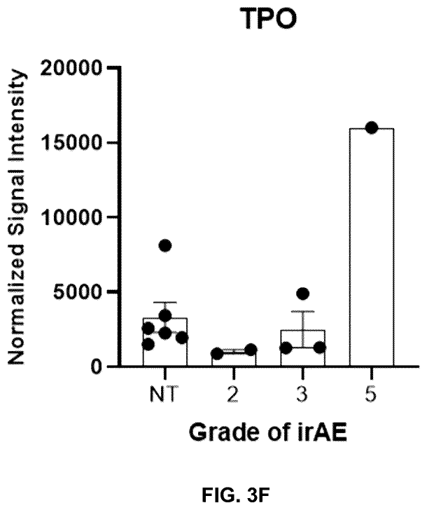

In some aspects, the disclosure further provides a profile which comprises one or more of a transcript profile, a cytokine profile, an autoantibody profile, an immune cell profile, or any combination thereof. The profile comprises one or more of the transcripts, autoantibodies, cytokines, and/or immune cells provided in Table 5, Table 6, Table 8, Table 9, Table 10, Table 11, FIGS. 2B-K, FIGS. 3B-F, FIG. 4A, respectively, or combinations thereof. In some aspects, the profile comprises a baseline or a pre-treatment transcript, cytokine, autoantibody, immune cell, or any combinations thereof, that correlates with future toxicity. In some aspects, one or more transcript, autoantibody, cytokine, immune cell subset, or any combination thereof are used as biomarkers to assess the risk of a subject developing irAE before, during, or after ICI treatment. In some aspects irAE is ICI-related myositis, ICI-related myocarditis or ICI-related myositis and myocarditis.

In some aspects, the method comprises providing a sample from the subject, assessing one or more transcript, autoantibody expression, cytokine expression, immune cell subset abundance, or any combination thereof, in the sample, and predicting risk for developing/diagnosing irAE in the subject. In some aspects, a subject can be predicted as having a higher risk for developing or diagnosed as having irAE associated with ICI treatment, when the profile comprises one or more transcript, autoantibody, cytokine, immune cell, or any combination thereof, at a greater level, expression, or abundance than control sample. In some aspects, a subject can be predicted as having a lower risk for developing or diagnosed as having irAE associated with ICI treatment, when the profile comprises one or more transcript, autoantibody, cytokine, immune cell, or any combination thereof, at a lower level, expression, or abundance than control sample.

In some aspects, the subject predicted or diagnosed with higher risk of developing irAE is associated with higher transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP531NP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C, GRASP, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL5, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, PD-L1+ naïve B cell, and/or switched memory B cell, compared to transcript level, expression, or abundance a control sample, at baseline, or before initiation of ICI treatment.

In some aspects, the subject predicted or diagnosed with higher risk of developing irAE is associated with higher transcript level, expression, or abundance of one or more of LILRB4, CISH, PARP9, RNF145, ASGR2, SLC16A13, LPAR6, GIMAP7, CXCR6, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and lower level, expression or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNA11, NOTCH3, ATF3, DUSP2, PER1, TNFSF9, MAFF, MIR4420, BRE-AS1, LOC102724428, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of CISH, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, TSC22D3, PD-L1+ naïve B cell, and/or switched memory B cell, compared to transcript level, expression, or abundance in a control sample, at baseline, or before initiation of ICI treatment.

In some aspects, the subject predicted or diagnosed with higher risk of developing irAE is associated with higher transcript level, expression, or abundance of one or more of PARP9, CISH, CXCR6, LPAR6, ASGR2 Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or lower level, expression or abundance of one or more KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, EGR1 PD-L1+ naïve B cell, and/or switched memory B cell, compared to transcript level, expression, or abundance in a control sample, at baseline, or before initiation of ICI treatment.

In some aspects, lower risk of developing irAE is associated with lower transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte or elevated transcript level, expression, or abundance of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C, GRASP, and/or PD-L1+ naïve B cell, and/or switched memory B cell.

In some aspects, the subject predicted or diagnosed with lower risk of developing irAE is associated with lower transcript level, expression, or abundance of one or more of LILRB4, CISH, PARP9, RNF145, ASGR2, SLC16A13, LPAR6, GIMAP7, CXCR6 Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with lower transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or higher transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or higher transcript level, expression, or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with lower transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or higher transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or higher transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with lower transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and higher transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with lower transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or higher transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNA11, NOTCH3, ATF3, DUSP2, PER1, TNFSF9, MAFF, MIR4420, BRE-AS1, LOC102724428, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with lower transcript level, expression, or abundance of one or more of CISH, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or higher transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with lower transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or higher transcript level, expression, or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with lower transcript level, expression, or abundance of one or more of PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or higher transcript level, expression, or abundance of one or more CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, TSC22D3, PD-L1+ naïve B cell, and/or switched memory B cell, compared to transcript level, expression, or abundance in a control sample, at baseline, or before initiation of ICI treatment.

In some aspects, the subject predicted or diagnosed with lower risk of developing irAE is associated with lower transcript level, expression, or abundance of one or more of PARP9, CISH, CXCR6, LPAR6, ASGR2 Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or higher transcript level, expression, or abundance of one or more KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, EGR1 PD-L1+ naïve B cell, and/or switched memory B cell, compared to transcript level, expression, or abundance in a control sample, at baseline, or before initiation of ICI treatment.

In some aspects, the disclosure further provides a profile for monitoring the risk of developing irAE which comprises one or more of a transcript profile, a cytokine profile, an autoantibody profile, an immune cell profile, or any combination thereof. The profile comprises one or more of the transcripts, autoantibodies, cytokines, and/or immune cells provided in Table 5, Table 6, Table 8, Table 9, Table 10, Table 11, FIGS. 2B-K, FIGS. 3B-F, FIG. 4A respectively, or combinations thereof. In some aspects, the profile comprises a baseline or a pre-treatment transcript, cytokine, autoantibody, immune cell, or any combinations thereof, that correlates with future toxicity. In some aspects, one or more transcript, autoantibody, cytokine, immune cell subset, or any combination thereof are used as biomarkers to assess the risk of a subject developing irAE before, during, or after ICI treatment. In some aspects, irAE is ICI-related myositis, ICI-related myocarditis or ICI-related myositis and myocarditis.

In some aspects, the method of monitoring the risk of irAE comprises providing a sample from the subject, assessing one or more transcript, autoantibody expression, cytokine expression, immune cell subset abundance, or any combination thereof, in the sample, and predicting risk for developing/diagnosing irAE in the subject. In some aspects, a subject being monitored can have a higher risk for irAE associated with ICI treatment, when the profile comprises one or more transcript, autoantibody, cytokine, immune cell, or any combination thereof, at a greater level, expression or abundance than control sample. In some aspects, a subject being monitored can have lower risk of irAE associated with ICI treatment, when the profile comprises one or more transcript, autoantibody, cytokine, immune cell, or any combination thereof, at a lower level, expression or abundance than control sample.

In some aspects, the subject being monitored can have higher risk of developing irAE when transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C, GRASP, PD-L1+ naïve B cell, and/or switched memory B cell is lower, compared to transcript level, expression, or abundance in a control sample, at baseline, or before initiation of ICI treatment.

In some aspects, the subject being monitored can have higher risk of developing irAE when transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, PD-L1+ naïve B cell, and/or switched memory B cell are lower, compared to transcript level, expression, or abundance in a control sample, at baseline, or before initiation of ICI treatment.

In some aspects, the subject being monitored can have higher risk of developing irAE when transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are lower, compared to transcript level, expression, or abundance in a control sample, at baseline, or before initiation of ICI treatment.

In some aspects, the subject being monitored can have higher risk of developing irAE when transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell are lower, compared to transcript level, expression, or abundance a control sample, at baseline, or before initiation of ICI treatment.

In some aspects, the subject being monitored can have higher risk of developing irAE when transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell are lower, compared to transcript level, expression, or abundance in a control sample, at baseline, or before initiation of ICI treatment.

In some aspects, the subject being monitored can have higher risk of developing irAE when transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are lower, compared to transcript level, expression, or abundance in a control sample, at baseline, or before initiation of ICI treatment.

In some aspects, the subject being monitored can have higher risk of developing irAE when transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are lower, compared to transcript level, expression, or abundance in a control sample, at baseline, or before initiation of ICI treatment.

In some aspects, the subject being monitored can have higher risk of developing irAE when transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNA11, NOTCH3, ATF3, DUSP2, PER1, TNFSF9, MAFF, MIR4420, BRE-AS1, LOC102724428, PD-L1+ naïve B cell, and/or switched memory B cell are lower, compared to transcript level, expression, or abundance in a control sample, at baseline, or before initiation of ICI treatment.

In some aspects, the subject being monitored can have higher risk of developing irAE when transcript level, expression, or abundance of one or more of CISH, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell are lower, compared to transcript level, expression, or abundance in a control sample, at baseline, or before initiation of ICI treatment.

In some aspects, the subject being monitored can have higher risk of developing irAE when transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell are lower, compared to transcript level, expression, or abundance in a control sample, at baseline, or before initiation of ICI treatment.

In some aspects, the subject being monitored can have higher risk of developing irAE when transcript level, expression, or abundance of one or more of PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte is higher and/or transcript level, expression, or abundance of one or more CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, TSC22D3, PD-L1+ naïve B cell, and/or switched memory B cell are lower, compared to transcript level, expression, or abundance a control sample, at baseline, or before initiation of ICI treatment.

In some aspects, the subject being monitored can have higher risk of developing irAE when transcript level, expression, or abundance of one or more of PARP9, CISH, CXCR6, LPAR6, ASGR2 Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or transcript level, expression, or abundance of one or more KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, EGR1 PD-L1+ naïve B cell, and/or switched memory B cell are lower, compared to transcript level, expression, or abundance in a control sample, at baseline, or before initiation of ICI treatment.

In some aspects, the subject being monitored can have lower risk of developing irAE when transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower or transcript level, expression, or abundance of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C, GRASP, PD-L1+ naïve B cell, and/or switched memory B cell are higher compared to transcript level, expression, or abundance in a control sample, at baseline, or before initiation of ICI treatment.

In some aspects, the subject being monitored can have lower risk of developing irAE when transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, PD-L1+ naïve B cell, and/or switched memory B cell are higher, compared to transcript level, expression, or abundance in a control sample, at baseline, or before initiation of ICI treatment.

In some aspects, the subject being monitored can have lower risk of developing irAE when transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are higher, compared to transcript level, expression, or abundance in a control sample, at baseline, or before initiation of ICI treatment.

In some aspects, the subject being monitored can have lower risk of developing irAE when transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or transcript level, expression, or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell are higher, compared to transcript level, expression, or abundance in a control sample, at baseline, or before initiation of ICI treatment.

In some aspects, the subject being monitored can have lower risk of developing irAE when, transcript level, expression, or abundance of one or more of LILRB4, CISH, PARP9, RNF145, ASGR2, SLC16A13, LPAR6, GIMAP7, CXCR6 Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell are higher, compared to transcript level, expression, or abundance in a control sample, at baseline, or before initiation of ICI treatment.

In some aspects, the subject being monitored can have lower risk of developing irAE when transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are higher, compared to transcript level, expression, or abundance in a control sample, at baseline, or before initiation of ICI treatment.

In some aspects, the subject being monitored can have lower risk of developing irAE when transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are higher, compared to transcript level, expression, or abundance in a control sample, at baseline, or before initiation of ICI treatment.

In some aspects, the subject being monitored can have lower risk of developing irAE when transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, PD-L1+ naïve B cell, and/or switched memory B cell are higher, compared to transcript level, expression, or abundance in a control sample, at baseline, or before initiation of ICI treatment.

In some aspects, the subject being monitored can have lower risk of developing irAE when transcript level, expression, or abundance of one or more of CISH, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell are higher, compared to transcript level, expression, or abundance in a control sample, at baseline, or before initiation of ICI treatment.

In some aspects, the subject being monitored can have lower risk of developing irAE when, transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or transcript level, expression, or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell is higher, compared to transcript level, expression, or abundance in a control sample, at baseline, or before initiation of ICI treatment.

In some aspects, the subject being monitored can have lower risk of developing irAE when transcript level, expression, or abundance of one or more of PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte is lower and/or transcript level, expression, or abundance of one or more CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, TSC22D3, PD-L1+ naïve B cell, and/or switched memory B cell are higher, compared to transcript level, expression, or abundance in a control sample, at baseline, or before initiation of ICI treatment.

In some aspects, the subject being monitored can have lower risk of developing irAE when transcript level, expression, or abundance of one or more of PARP9, CISH, CXCR6, LPAR6, ASGR2 Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or transcript level, expression, or abundance of one or more KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, EGR1 PD-L1+ naïve B cell, and/or switched memory B cell are higher, compared to transcript level, expression, or abundance in a control sample, at baseline, or before initiation of ICI treatment.

### Methods of Treatments

The present disclosure further provides a method of treating a subject with cancer. The method comprises assessing one or more of the transcript, autoantibody, cytokine, immune cell profile or any combinations thereof and assigning a risk level for developing irAE. In some aspects, risk assessment is done before, or during ICI treatment. In some aspects, the risk assessment comprises predicting, diagnosing, or monitoring ICI-related myositis, ICI-related myocarditis or ICI-related myositis and myocarditis. If the profile correlates with irAE or future development of irAE the subjects are assigned high risk for developing irAE. If the profile correlates with no irAE the subjects are assigned a low risk for developing irAE.

In some aspects, the ICI treatment comprises one or more ICI treatment disclosed herein. The progression of treatment of cancer using ICI is guided by the profile assessments and assigned risk levels. Based on the profile assessments and assigned risk levels, the ICI treatment can be continued, withdrawn, or modified accordingly.

In some aspects, the method for treating cancer comprises providing a sample from a subject, assessing one or more of the transcript, autoantibody, cytokine, immune cell subsets or any combinations thereof, treating the subject with an ICI treatment if subject is predicted, diagnosed, or monitored as low risk for irAE. In some aspects, the subject receives a non-ICI treatment if the subject is predicted, diagnosed or monitored as high risk. In some aspects, the subject receives an ICI treatment and an irAE mitigating treatment if the subject is diagnosed as high risk.

In some aspects, ICI comprises administration of an inhibitor of PD-1, PD-L1, TIM-3, LAG-3, CTLA-4, CSF-1R, or any combinations thereof. In some aspects, irAE is ICI-related myositis, ICI-related myocarditis or ICI-related myositis and myocarditis. In some aspects, the irAE mitigating therapy is selected from corticosteroids (e.g., prednisone, methylprednisolone, dexamethasone, budesonide), TNF inhibitors (e.g., infliximab), or hormone replacement (e.g., hydrocortisone, levothyroxine), CXCL8 inhibitors (e.g. repertaxin).

In some aspects, based on the severity of irAE, ICI treatment may be suspended, with consideration of resuming when symptoms of irAE revert. In some aspects, the dosage of ICI treatment may be reduced or skipped. Additionally, irAE mitigating therapy for e.g., corticosteroids may be administered. Subjects may be administered a high-dose corticosteroids (for e.g., prednisone 1 to 2 mg/kg/d or methylprednisolone 1 to 2 mg/kg/d), which may be tapered over the course of at least 4 to 6 weeks. In some aspects, infliximab or other immunosuppressive therapy may be administered either individually, or in combination with other irAE mitigating therapies. In some aspects, irAE mitigating therapy may be administered sequentially or simultaneously with the ICI treatment. In some aspects, permanent discontinuation of ICI may be recommended. In some aspects, a non-ICI treatment may be recommended.

In some aspects, corticosteroids include, for example, betamethasone sodium phosphate, desonide sodium phosphate, dexamethasone sodium phosphate, hydrocortisone sodium phosphate, hydrocortisone sodium succinate, methylprednisolone disodium phosphate, methylprednisolone sodium succinate, prednisolone sodium phosphate, prednisolone sodium succinate, prednisolamate hydrochloride, prednisone disodium phosphate, prednisone sodium succinate, triamcinolone acetonide disodium phosphate and triamcinolone acetonide disodium phosphate, alclomethasone dipropionate, amcinonide, beclomethasone monopropionate, betamethasone 17-valerate, ciclomethasone, clobetasol propionate, clobetasone butyrate, deprodone propionate, desonide, desoxymethasone, dexamethasone acetate, diflucortolone valerate, diflurasone diacetate, diflucortolone, difluprednate, flumetasone pivalate, flunisolide, fluocinolone acetonide acetate, fluocinonide, fluocortolone pivalate, fluormetholone acetate, fluprednidene acetate, halcinonide, halometasone, hydrocortisone acetate, medrysone, methylprednisolone acetate, mometasone furoate, parametasone acetate, prednicarbate, prednisolone acetate, prednylidene, rimexolone, tixocortol pivalate and triamcinolone hexacetonide.

In some aspects, non-ICI therapy include chemotherapy, hormonal therapy, small molecule therapy, toxin therapy, prodrug-activating enzyme therapy, biologic therapy, surgical therapy, anti-angiogenic therapy, targeted therapy, epigenetic therapy, demethylation therapy, histone deacetylase inhibitor therapy, differentiation therapy, radiation therapy, stem cell transplantation and/or any combination thereof.

Cancer therapeutic agents or chemotherapeutic agents include alkylating agents such as thiotepa and cyclophosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL™, Bristol-Myers Squibb Oncology, Princeton, N.J.) and doxetaxel (TAXOTEPvE™, Pvhne-Poulenc Rorer, Antony, France); chlorambucil; gemcitabine; 6-thioguanine; mercaptopurine; methotrexate; platinum analogs such as cisplatin and carboplatin; vinblastine; trastuzumab, docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid derivatives such as Targretin™ (bexarotene), Panretin™ (alitretinoin); ONTAKT™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 1 17018, onapristone, and toremifene (Fareston); and anti-androgens such as flutamide, nilutamide, bicalutamide, leuprolide, and goserelin; and pharmaceutically acceptable salts, acids or derivatives of any of the above. Further cancer therapeutic agents include sorafenib and other protein kinase inhibitors such as afatinib, axitinib, bevacizumab, cetuximab, crizotinib, dasatinib, erlotinib, fostamatinib, gefitinib, imatinib, lapatinib, lenvatinib, mubritinib, nilotinib, panitumumab, pazopanib, pegaptanib, ranibizumab, ruxolitinib, trastuzumab, vandetanib, vemurafenib, and sunitinib; sirolimus (rapamycin), everolimus and other mTOR inhibitors. Examples of additional chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea). Moreover, exemplary chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase.

In some aspects, the subject is treated with a non-ICI treatment, or an ICI treatment and an irAE mitigating treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to transcript levels in a control sample.

In some aspects, the subject is treated with a non-ICI treatment, or an ICI treatment and an irAE mitigating treatment if the transcript levels of one or more of LILRB4, CISH and/or PARP9 is elevated prior to ICI treatment (baseline), compared to transcript levels in a control sample. In some aspects, the subject is treated with a non-ICI treatment, or an ICI treatment and an irAE mitigating treatment if the transcript levels of one or more of LILRB4, CISH, GIMAP7, CXCR6, DHRS9, FCGR1CP, and/or ANKRD34B is elevated prior to ICI treatment (baseline), compared to transcript levels in a control sample.

In some aspects, the subject is treated with a non-ICI treatment, or an ICI treatment and an irAE mitigating treatment if the transcript level of LILRB4 is elevated prior to ICI treatment, compared to the transcript level in a control sample. In some aspects, the subject is treated with a non-ICI treatment, or an ICI treatment and an irAE mitigating treatment if the transcript level of CISH is elevated prior to ICI treatment, compared to the transcript level in a control sample. In some aspects, the subject is treated with a non-ICI treatment, or an ICI treatment and an irAE mitigating treatment if the transcript level of PARP9 is elevated prior to ICI treatment, compared to the transcript level in a control sample. In some aspects, the subject is treated with a non-ICI treatment, or an ICI treatment and an irAE mitigating treatment if the transcript level of RNF145 is elevated prior to ICI treatment, compared to the transcript level in a control sample. In some aspects, the subject is treated with a non-ICI treatment, or an ICI treatment and an irAE mitigating treatment if the transcript level of ASGR2 is elevated prior to ICI treatment, compared to the transcript level in a control sample. In some aspects, the subject is treated with a non-ICI treatment, or an ICI treatment and an irAE mitigating treatment if the transcript level of SLC16A13 is elevated prior to ICI treatment, compared to the transcript level in a control sample. In some aspects, the subject is treated with a non-ICI treatment, or an ICI treatment and an irAE mitigating treatment if the transcript level of LPAR6 is elevated prior to ICI treatment, compared to the transcript level in a control sample. In some aspects, the subject is treated with a non-ICI treatment, or an ICI treatment and an irAE mitigating treatment if the transcript level of GIMAP7 is elevated prior to ICI treatment, compared to the transcript level in a control sample. In some aspects, the subject is treated with a non-ICI treatment, or an ICI treatment and an irAE mitigating treatment if the transcript level of CXCR6 is elevated prior to ICI treatment, compared to the transcript level in a control sample. In some aspects, the subject is treated with a non-ICI treatment, or an ICI treatment and an irAE mitigating treatment if the transcript level of DHRS9 is elevated prior to ICI treatment, compared to the transcript level in a control sample. In some aspects, the subject is treated with a non-ICI treatment, or an ICI treatment and an irAE mitigating treatment if the transcript level of FCGR1CP is elevated prior to ICI treatment, compared to the transcript level in a control sample. In some aspects, the subject is treated with a non-ICI treatment, or an ICI treatment and an irAE mitigating treatment if the transcript level of ANKRD34B is elevated prior to ICI treatment, compared to the transcript level in a control sample.

In some aspects, the subject is treated with a non-ICI treatment, or an ICI treatment and an irAE mitigating treatment if the transcript level of at least LILRB4 is elevated in the subject prior to ICI treatment compared to the transcript level in a control sample. In some aspects, the subject is treated with a non-ICI treatment, or an ICI treatment and an irAE mitigating treatment if the transcript level of at least CISH is elevated in the subject prior to ICI treatment compared to the transcript level in a control sample. In some aspects, the subject is treated with a non-ICI treatment, or an ICI treatment and an irAE mitigating treatment if the transcript level of at least PARP9 is elevated prior to ICI treatment, compared to the transcript level in a control sample. In some aspects, the subject is treated with a non-ICI treatment, or an ICI treatment and an irAE mitigating treatment if the transcript level of at least GIMAP7 is elevated in the subject prior to ICI treatment compared to the transcript level in a control sample. In some aspects, the subject is treated with a non-ICI treatment, or an ICI treatment and an irAE mitigating treatment if the transcript level of at least CXCR6 is elevated in the subject prior to ICI treatment compared to the transcript level in a control sample. In some aspects, the subject is treated with a non-ICI treatment, or an ICI treatment and an irAE mitigating treatment if the transcript level of at least DHRS9 is elevated prior to ICI treatment, compared to the transcript level in a control sample. In some aspects, the subject is treated with a non-ICI treatment, or an ICI treatment and an irAE mitigating treatment if the transcript level of at least FCGR1CP is elevated prior to ICI treatment, compared to the transcript level in a control sample. In some aspects, the subject is treated with a non-ICI treatment, or an ICI treatment and an irAE mitigating treatment if the transcript level of at least ANKRD34B is elevated prior to ICI treatment, compared to the transcript level in a control sample.

In some aspects, the subject is treated with a non-ICI treatment, or an ICI treatment and an irAE mitigating treatment if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP is lower in the subject when compared to the transcript levels in a control sample.

In some aspects, the subject is treated with a non-ICI treatment, or an ICI treatment and an irAE mitigating treatment if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP is lower in the subject when compared to the transcript levels in a control sample.

In some aspects, the subject is treated with a non-ICI treatment, or an ICI treatment and an irAE mitigating treatment if the transcript levels of one or more of AREG, EREG, OSM, CSRNP1, DDIT4, IL-10, PTGS2, DUSP1, CXCR4, NFIL3, FOS, NFKBIA, PPP1R15A, CD79A and/or JUNB is lower prior to ICI treatment, compared to the transcript levels in a control sample. In various aspects, the subject is treated with a non-ICI treatment, or an ICI treatment and an irAE mitigating treatment if the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 is lower prior to ICI treatment, compared to the transcript levels in a control sample. In some other aspects, the subject is treated with a non-ICI treatment, or an ICI treatment and an irAE mitigating treatment if the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 is lower prior to ICI treatment, compared to the transcript levels in a control sample.

In some aspects, the subject is treated with a non-ICI treatment, or an ICI treatment and an irAE mitigating treatment if the transcript level of at least AREG is lower in the subject when compared to the transcript level in a control sample. In some aspects, the subject is treated with a non-ICI treatment, or an ICI treatment and an irAE mitigating treatment if the transcript level of at least EREG is lower in the subject when compared to the transcript level in a control sample. In some aspects, the subject is treated with a non-ICI treatment, or an ICI treatment and an irAE mitigating treatment if the transcript level of at least OSM is lower in the subject when compared to the transcript level in a control sample. In some aspects, the subject is treated with a non-ICI treatment, or an ICI treatment and an irAE mitigating treatment if the transcript level of at least CSRNP1 is lower in the subject when compared to the transcript level in a control sample. In some aspects, the subject is treated with a non-ICI treatment, or an ICI treatment and an irAE mitigating treatment if the transcript level of at least DDIT4 is lower in the subject when compared to the transcript level in a control sample. In some aspects, the subject is treated with a non-ICI treatment, or an ICI treatment and an irAE mitigating treatment if the transcript level of at least IL-10 is lower in the subject when compared to the transcript level in a control sample. In some aspects, the subject is treated with a non-ICI treatment, or an ICI treatment and an irAE mitigating treatment if the transcript level of at least PTGS2 is lower in the subject when compared to the transcript level in a control sample. In some aspects, the subject is treated with a non-ICI treatment, or an ICI treatment and an irAE mitigating treatment if the transcript level of at least DUSP1 is lower in the subject when compared to the transcript level in a control sample. In some aspects, the subject is treated with a non-ICI treatment, or an ICI treatment and an irAE mitigating treatment if the transcript level of at least CXCR4 is lower in the subject when compared to the transcript level in a control sample. In some aspects, the subject is treated with a non-ICI treatment, or an ICI treatment and an irAE mitigating treatment if the transcript level of at least NFIL3 is lower in the subject when compared to the transcript level in a control sample. In some aspects, the subject is treated with a non-ICI treatment, or an ICI treatment and an irAE mitigating treatment if the transcript level of at least FOS is lower in the subject when compared to the transcript level in a control sample. In some aspects, the subject is treated with a non-ICI treatment, or an ICI treatment and an irAE mitigating treatment if the transcript level of at least NFKBIA is lower in the subject when compared to the transcript level in a control sample. In some aspects, the subject is treated with a non-ICI treatment, or an ICI treatment and an irAE mitigating treatment if the transcript level of at least PPP1R15A is lower in the subject when compared to the transcript level in a control sample. In some aspects, the subject is treated with a non-ICI treatment, or an ICI treatment and an irAE mitigating treatment if the transcript level of at least CD79A is lower in the subject when compared to the transcript level in a control sample. In some aspects, the subject is treated with a non-ICI treatment, or an ICI treatment and an irAE mitigating treatment if the transcript level of at least JUNB is lower in the subject when compared to the transcript level in a control sample. In some aspects, the subject is treated with a non-ICI treatment, or an ICI treatment and an irAE mitigating treatment if the transcript level of at least CXCL8 is lower in the subject when compared to the transcript level in a control sample. In some aspects, the subject is treated with a non-ICI treatment, or an ICI treatment and an irAE mitigating treatment if the transcript level of at least EGR1 is lower in the subject when compared to the transcript level in a control sample. In some aspects, the subject is treated with a non-ICI treatment, or an ICI treatment and an irAE mitigating treatment if the transcript level of at least G0S2 is lower in the subject when compared to the transcript level in a control sample. In some aspects, the subject is treated with a non-ICI treatment, or an ICI treatment and an irAE mitigating treatment if the transcript level of at least PAX8 is lower in the subject when compared to the transcript level in a control sample. In some aspects, the subject is treated with a non-ICI treatment, or an ICI treatment and an irAE mitigating treatment if the transcript level of at least ATF6B is lower in the subject when compared to the transcript level in a control sample. In some aspects, the subject is treated with a non-ICI treatment, or an ICI treatment and an irAE mitigating treatment if the transcript level of at least PAX8-AS1 is lower in the subject when compared to the transcript level in a control sample. In some aspects, the subject is treated with a non-ICI treatment, or an ICI treatment and an irAE mitigating treatment if the transcript level of at least RNVU1-19 is lower in the subject when compared to the transcript level in a control sample. In some aspects, the subject is treated with a non-ICI treatment, or an ICI treatment and an irAE mitigating treatment if the transcript level of at least VMO1 is lower in the subject when compared to the transcript level in a control sample. In some aspects, the subject is treated with a non-ICI treatment, or an ICI treatment and an irAE mitigating treatment if the transcript level of at least HBEGF is lower in the subject when compared to the transcript level in a control sample. In some aspects, the subject is treated with a non-ICI treatment, or an ICI treatment and an irAE mitigating treatment if the transcript level of at least CCDC144A is lower in the subject when compared to the transcript level in a control sample. In some aspects, the subject is treated with a non-ICI treatment, or an ICI treatment and an irAE mitigating treatment if the transcript level of at least SHISA8 is lower in the subject when compared to the transcript level in a control sample. In some aspects, the subject is treated with a non-ICI treatment, or an ICI treatment and an irAE mitigating treatment if the transcript level of at least NR4A2 is lower in the subject when compared to the transcript level in a control sample. In some aspects, the subject is treated with a non-ICI treatment, or an ICI treatment and an irAE mitigating treatment if the transcript level of at least PTGES is lower in the subject when compared to the transcript level in a control sample. In some aspects, the subject is treated with a non-ICI treatment, or an ICI treatment and an irAE mitigating treatment if the transcript level of at least SYN1 is lower in the subject when compared to the transcript level in a control sample. In some aspects, the subject is treated with a non-ICI treatment, or an ICI treatment and an irAE mitigating treatment if the transcript level of at least CXCL2 is lower in the subject when compared to the transcript level in a control sample. In some aspects, the subject is treated with a non-ICI treatment, or an ICI treatment and an irAE mitigating treatment if the transcript level of at least PMP22 is lower in the subject when compared to the transcript level in a control sample. In some aspects, the subject is treated with a non-ICI treatment, or an ICI treatment and an irAE mitigating treatment if the transcript level of at least CD83 is lower in the subject when compared to the transcript level in a control sample. In some aspects, the subject is treated with a non-ICI treatment, or an ICI treatment and an irAE mitigating treatment if the transcript level of at least EGR3 is lower in the subject when compared to the transcript level in a control sample. In some aspects, the subject is treated with a non-ICI treatment, or an ICI treatment and an irAE mitigating treatment if the transcript level of at least NUAK1 is lower in the subject when compared to the transcript level in a control sample. In some aspects, the subject is treated with a non-ICI treatment, or an ICI treatment and an irAE mitigating treatment if the transcript level of at least NOCT is lower in the subject when compared to the transcript level in a control sample. In some aspects, the subject is treated with a non-ICI treatment, or an ICI treatment and an irAE mitigating treatment if the transcript level of at least ATOH8 is lower in the subject when compared to the transcript level in a control sample. In some aspects, the subject is treated with a non-ICI treatment, or an ICI treatment and an irAE mitigating treatment if the transcript level of at least PLK2 is lower in the subject when compared to the transcript level in a control sample. In some aspects, the subject is treated with a non-ICI treatment, or an ICI treatment and an irAE mitigating treatment if the transcript level of at least ID1 is lower in the subject when compared to the transcript level in a control sample. In some aspects, the subject is treated with a non-ICI treatment, or an ICI treatment and an irAE mitigating treatment if the transcript level of at least ADRB1 is lower in the subject when compared to the transcript level in a control sample. In some aspects, the subject is treated with a non-ICI treatment, or an ICI treatment and an irAE mitigating treatment if the transcript level of at least SNAI1 is lower in the subject when compared to the transcript level in a control sample. In some aspects, the subject is treated with a non-ICI treatment, or an ICI treatment and an irAE mitigating treatment if the transcript level of at least NOTCH3 is lower in the subject when compared to the transcript level in a control sample. In some aspects, the subject is treated with a non-ICI treatment, or an ICI treatment and an irAE mitigating treatment if the transcript level of at least ATF3 is lower in the subject when compared to the transcript level in a control sample. In some aspects, the subject is treated with a non-ICI treatment, or an ICI treatment and an irAE mitigating treatment if the transcript level of at least DUSP2 is lower in the subject when compared to the transcript level in a control sample. In some aspects, the subject is treated with a non-ICI treatment, or an ICI treatment and an irAE mitigating treatment if the transcript level of at least PER1 is lower in the subject when compared to the transcript level in a control sample. In some aspects, the subject is treated with a non-ICI treatment, or an ICI treatment and an irAE mitigating treatment if the transcript level of at least TNFSF9 is lower in the subject when compared to the transcript level in a control sample. In some aspects, the subject is treated with a non-ICI treatment, or an ICI treatment and an irAE mitigating treatment if the transcript level of at least MAFF is lower in the subject when compared to the transcript level in a control sample. In some aspects, the subject is treated with a non-ICI treatment, or an ICI treatment and an irAE mitigating treatment if the transcript level of at least MI R4420 is lower in the subject when compared to the transcript level in a control sample. In some aspects, the subject is treated with a non-ICI treatment, or an ICI treatment and an irAE mitigating treatment if the transcript level of at least BRE-AS1 is lower in the subject when compared to the transcript level in a control sample. In some aspects, the subject is treated with a non-ICI treatment, or an ICI treatment and an irAE mitigating treatment if the transcript level of at least LOC102724428 is lower in the subject when compared to the transcript level in a control sample. In some aspects, the subject is treated with a non-ICI treatment, or an ICI treatment and an irAE mitigating treatment if the transcript level of at least GRASP is lower in the subject when compared to the transcript level in a control sample. In some aspects, the subject is treated with a non-ICI treatment, or an ICI treatment and an irAE mitigating treatment if the transcript level of at least TNFAIP3 is lower in the subject when compared to the transcript level in a control sample. In some aspects, the subject is treated with a non-ICI treatment, or an ICI treatment and an irAE mitigating treatment if the transcript level of at least TRIB1 is lower in the subject when compared to the transcript level in a control sample. In some aspects, the subject is treated with a non-ICI treatment, or an ICI treatment and an irAE mitigating treatment if the transcript level of at least PMAIP1 is lower in the subject when compared to the transcript level in a control sample. In some aspects, the subject is treated with a non-ICI treatment, or an ICI treatment and an irAE mitigating treatment if the transcript level of at least FAM46C is lower in the subject when compared to the transcript level in a control sample. In some aspects, the subject is treated with a non-ICI treatment, or an ICI treatment and an irAE mitigating treatment if the transcript level of at least NXT1 is lower in the subject when compared to the transcript level in a control sample. In some aspects, the subject is treated with a non-ICI treatment, or an ICI treatment and an irAE mitigating treatment if the transcript level of at least KLF9 is lower in the subject when compared to the transcript level in a control sample.

In some aspects, the subject is treated with a non-ICI treatment, or an ICI treatment and an irAE mitigating treatment if the transcript level of AREG is lower prior to ICI treatment, compared to the transcript level in a control sample. In some aspects, the subject is treated with a non-ICI treatment, or an ICI treatment and an irAE mitigating treatment if the transcript level of EREG is lower prior to ICI treatment, compared to the transcript level in a control sample. In some aspects, the subject is treated with a non-ICI treatment, or an ICI treatment and an irAE mitigating treatment if the transcript level of OSM is lower prior to ICI treatment, compared to the transcript level in a control sample. In some aspects, the subject is treated with a non-ICI treatment, or an ICI treatment and an irAE mitigating treatment if the transcript level of CSRNP1 is lower prior to ICI treatment, compared to the transcript level in a control sample. In some aspects, the subject is treated with a non-ICI treatment, or an ICI treatment and an irAE mitigating treatment if the transcript level of DDIT4 is lower prior to ICI treatment, compared to the transcript level in a control sample. In some aspects, the subject is treated with a non-ICI treatment, or an ICI treatment and an irAE mitigating treatment if the transcript level of IL-10 is lower prior to ICI treatment, compared to the transcript level in a control sample. In some aspects, the subject is treated with a non-ICI treatment, or an ICI treatment and an irAE mitigating treatment if the transcript level of PTGS2 is lower prior to ICI treatment, compared to the transcript level in a control sample. In some aspects, the subject is treated with a non-ICI treatment, or an ICI treatment and an irAE mitigating treatment if the transcript level of DUSP1 is lower prior to ICI treatment, compared to the transcript level in a control sample. In some aspects, the subject is treated with a non-ICI treatment, or an ICI treatment and an irAE mitigating treatment if the transcript level of CXCR4 is lower prior to ICI treatment, compared to the transcript level in a control sample. In some aspects, the subject is treated with a non-ICI treatment, or an ICI treatment and an irAE mitigating treatment if the transcript level of NIFL3 is lower prior to ICI treatment, compared to the transcript level in a control sample. In some aspects, the subject is treated with a non-ICI treatment, or an ICI treatment and an irAE Mitigating treatment if the transcript level of FOS is lower prior to ICI treatment, compared to the transcript level in a control sample. In some aspects, the subject is treated with a non-ICI treatment, or an ICI treatment and an irAE mitigating treatment if the transcript level of NFKBIA is lower prior to ICI treatment, compared to the transcript level in a control sample. In some aspects, the subject is treated with a non-ICI treatment, or an ICI treatment and an irAE mitigating treatment if the transcript level of PPP1R15A is lower prior to ICI treatment, compared to the transcript level in a control sample. In some aspects, the subject is treated with a non-ICI treatment, or an ICI treatment and an irAE mitigating treatment if the transcript level of CD79A is lower prior to ICI treatment, compared to the transcript level in a control sample. In some aspects, the subject is treated with a non-ICI treatment, or an ICI treatment and an irAE mitigating treatment if the transcript level of JUNB is lower prior to ICI treatment, compared to the transcript level in a control sample.

In some aspects, the subject is treated with a non-ICI treatment, or an ICI treatment and an irAE mitigating treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are lower in the subject when compared to the transcript levels in a control sample.

In some aspects, the subject is treated with a non-ICI treatment, or an ICI treatment and an irAE mitigating treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are lower in the subject when compared to the transcript levels in a control sample.

In some aspects, the subject is treated with a non-ICI treatment, or an ICI treatment and an irAE mitigating treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more transcript of AREG, EREG, OSM, CSRNP1, DDIT4, IL-10, PTGS2, DUSP1, CXCR4, NFIL3, FOS, NFKBIA, PPP1R15A, CD79A, and/or JUNB are lower in the subject when compared to the transcript levels in a control sample.

In some aspects, the subject is treated with a non-ICI treatment, or an ICI treatment and an irAE mitigating treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 is lower in the subject when compared to the transcript levels in a control sample.

In some aspects, the subject is treated with a non-ICI treatment, or an ICI treatment and an irAE mitigating treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are elevated prior to ICI treatment (baseline), compared to the transcript level in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 is lower in the subject when compared to the transcript levels in a control sample.

In some aspects, the subject is treated with a non-ICI treatment, or an ICI treatment and an irAE mitigating treatment if the transcript levels of one or more of LILRB4, CISH, and/or PARP9 are elevated prior to ICI treatment (baseline), compared to the transcript level in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB is lower in the subject when compared to the transcript levels in a control sample.

In some aspects, the subject is treated with a non-ICI treatment, or an ICI treatment and an irAE mitigating treatment if the transcript levels of LILRB4, CISH, and PARP9 are elevated prior to ICI treatment (baseline), compared to the transcript levels in a control sample; and the transcript levels of AREG, EREG, OSM, CSRNP1, DDIT4, IL-10, PTGS2, DUSP1, CXCR4, NFIL3, FOS, NFKBIA, PPP1R15A, CD79A, and JUNB are lower in the subject when compared to the transcript levels in a control sample.

In some aspects, the subject is treated with a non-ICI treatment, or an ICI treatment and an irAE mitigating treatment if the transcript levels of one or more of LILRB4, GIMAP7, CISH, CXCR6, DHRS9, FCGR1CP, and/or ANKRD34B are elevated prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are lower in the subject when compared to the transcript levels in a control sample.

In some aspects, the subject is treated with a non-ICI treatment, or an ICI treatment and an irAE mitigating treatment if the transcript level of CISH is elevated prior to ICI treatment, compared to the transcript level in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower in the subject when compared to the transcript levels in a control sample.

In some aspects, the subject is treated with a non-ICI treatment, or an ICI treatment and an irAE mitigating treatment if the transcript levels of one or more of LILRB4, GIMAP7, CISH, CXCR6, DHRS9, FCGR1CP, and/or ANKRD34B are elevated prior to ICI treatment (baseline), compared to the transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are lower in the subject when compared to the transcript levels in a control sample.

In some aspects, the subject is treated with a non-ICI treatment, or an ICI treatment and an irAE mitigating treatment if the transcript level of the PARP9 is elevated prior to ICI treatment (baseline), compared to the transcript levels in a control sample; and the transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are lower in the subject when compared to the level in a control sample.

In some aspects, the subject is treated with a non-ICI treatment, or an ICI treatment and an irAE mitigating treatment if the transcript levels of one or more of PARP9, CISH, CXCR6, LPAR6, and/or ASGR2 are elevated prior to ICI treatment (baseline), compared to the transcript levels in a control sample; and the transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKB1A, TNFS9, NUAK1, ATOH8, TNFAIP3, ADRB1, HBEGF, IL-10, JUND, FOS, GABARAPL1, PMAIP1, DDIT4, and/or EGR1 are lower in the subject when compared to the transcript levels in a control sample.

In some aspects, the subject is treated with an ICI treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to the transcript levels in a control sample.

In some aspects, the subject is treated with an ICI treatment if the transcript levels of one or more of LILRB4, CISH and/or PARP9 are elevated prior to ICI treatment (baseline), compared to the transcript levels in a control sample. In some aspects, the subject is treated with an ICI treatment if the transcript levels of one or more transcript of LILRB4, CISH, GIMAP7, CXCR6, DHRS9, FCGR1CP, and/or ANKRD34B are lower prior to ICI treatment (baseline), compared to the transcript levels in a control sample.

In some aspects, the subject is treated with an ICI treatment if the transcript level of LILRB4 is lower prior to ICI treatment, compared to the transcript level in a control sample. In some aspects, the subject is treated with an ICI treatment if the transcript level of CISH is lower prior to ICI treatment, compared to the transcript level in a control sample. In some aspects, the subject is treated with an ICI treatment if the transcript level of PARP9 is lower prior to ICI treatment, compared to the transcript level in a control sample. In some aspects, the subject is treated with an ICI treatment if the transcript level of RNF145 is lower prior to ICI treatment, compared to the transcript level in a control sample. In some aspects, the subject is treated with an ICI treatment if the transcript level of ASGR2 is lower prior to ICI treatment, compared to the transcript level in a control sample. In some aspects, the subject is treated with an ICI treatment if the transcript level of SLC16A13 is lower prior to ICI treatment, compared to the transcript level in a control sample. In some aspects, the subject is treated with an ICI treatment if the transcript level of LPAR6 is lower prior to ICI treatment, compared to the transcript level in a control sample. In some aspects, the subject is treated with an ICI treatment if the transcript level of GIMAP7 is lower prior to ICI treatment, compared to the transcript level in a control sample. In some aspects, the subject is treated with an ICI treatment if the transcript level of CXCR6 is lower prior to ICI treatment, compared to the transcript level in a control sample. In some aspects, the subject is treated with an ICI treatment if the transcript level of DHRS9 is lower prior to ICI treatment, compared to the transcript level in a control sample. In some aspects, the subject is treated with an ICI treatment if the transcript level of FCGR1CP is lower prior to ICI treatment, compared to the transcript level in a control sample. In some aspects, the subject is treated with an ICI treatment if the transcript level of ANKRD34B is lower prior to ICI treatment, compared to the transcript level in a control sample.

In some aspects, the subject is treated with an ICI treatment if the transcript level of at least LILRB4 is lower in the subject prior to ICI treatment compared to the transcript level in a control sample. In some aspects, the subject is treated with an ICI treatment if the transcript level of at least CISH is lower in the subject prior to ICI treatment compared to the transcript level in a control sample. In some aspects, the subject is treated with an ICI treatment if the transcript level of at least PARP9 is lower prior to ICI treatment, compared to the transcript level in a control sample. In some aspects, the subject is treated with an ICI treatment if the transcript level of at least GIMAP7 is lower in the subject prior to ICI treatment compared to the transcript level in a control sample. In some aspects, the subject is treated with an ICI treatment if the transcript level of at least CXCR6 is lower in the subject prior to ICI treatment compared to the transcript level in a control sample. In some aspects, the subject is treated with an ICI treatment if the transcript level of at least DHRS9 is lower prior to ICI treatment, compared to the transcript level in a control sample. In some aspects, the subject is treated with an ICI treatment if the transcript level of at least FCGR1CP is lower prior to ICI treatment, compared to the transcript level in a control sample. In some aspects, the subject is treated with an ICI treatment if the transcript level of at least ANKRD34B is lower prior to ICI treatment, compared to the transcript level in a control sample.

In some aspects, the subject is treated with an ICI treatment if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP531NP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are elevated in the subject when compared to the transcript levels in a control sample.

In some aspects, the subject is treated with an ICI treatment if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP are elevated in the subject when compared to the transcript levels in a control sample.

In some aspects, the subject is treated with an ICI treatment if the transcript levels of one or more of AREG, EREG, OSM, CSRNP1, DDIT4, IL-10, PTGS2, DUSP1, CXCR4, NFIL3, FOS, NFKBIA, PPP1R15A, CD79A and/or JUNB are elevated prior to ICI treatment, compared to the transcript levels in a control sample. In various aspects, the subject is treated with an ICI treatment if the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are elevated prior to ICI treatment, compared to the transcript levels in a control sample. In some other aspects, the subject is treated with an ICI treatment if the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are elevated prior to ICI treatment, compared to the transcript levels in a control sample.

In some aspects, the subject is treated with an ICI treatment if the transcript level of at least AREG is elevated in the subject when compared to the transcript level in a control sample. In some aspects, the subject is treated with an ICI treatment if the transcript level of at least EREG is elevated in the subject when compared to the transcript level in a control sample. In some aspects, the subject is treated with an ICI treatment if the transcript level of at least OSM is elevated in the subject when compared to the transcript level in a control sample. In some aspects, the subject is treated with an ICI treatment if the transcript level of at least CSRNP1 is elevated in the subject when compared to the transcript level in a control sample. In some aspects, the subject is treated with an ICI treatment if the transcript level of at least DDIT4 is elevated in the subject when compared to the transcript level in a control sample. In some aspects, the subject is treated with an ICI treatment if the transcript level of at least IL-10 is elevated in the subject when compared to the transcript level in a control sample. In some aspects, the subject is treated with an ICI treatment if the transcript level of at least PTGS2 is elevated in the subject when compared to the transcript level in a control sample. In some aspects, the subject is treated with an ICI treatment if the transcript level of at least DUSP1 is elevated in the subject when compared to the transcript level in a control sample. In some aspects, the subject is treated with an ICI treatment if the transcript level of at least CXCR4 is elevated in the subject when compared to the transcript level in a control sample. In some aspects, the subject is treated with an ICI treatment if the transcript level of at least NFIL3 is elevated in the subject when compared to the transcript level in a control sample. In some aspects the subject is treated with an ICI treatment if the transcript level of at least FOS is elevated in the subject when compared to the transcript level in a control sample. In some aspects, the subject is treated with an ICI treatment if the transcript level of at least NFKBIA is elevated in the subject when compared to the transcript level in a control sample. In some aspects, the subject is treated with an ICI treatment if the transcript level of at least PPP1R15A is elevated in the subject when compared to the transcript level in a control sample. In some aspects, the subject is treated with an ICI treatment if the transcript level of at least CD79A is elevated in the subject when compared to the transcript level in a control sample. In some aspects, the subject is treated with an ICI treatment if the transcript level of at least JUNB is elevated in the subject when compared to the transcript level in a control sample. In some aspects, the subject is treated with an ICI treatment if the transcript level of at least CXCL8 is elevated in the subject when compared to the transcript level in a control sample. In some aspects, the subject is treated with an ICI treatment if the transcript level of at least EGR1 is elevated in the subject when compared to the transcript level in a control sample. In some aspects, the subject is treated with an ICI treatment if the transcript level of at least G0S2 is elevated in the subject when compared to the transcript level in a control sample. In some aspects, the subject is treated with an ICI treatment if the transcript level of at least PAX8 is elevated in the subject when compared to the transcript level in a control sample. In some aspects, the subject is treated with an ICI treatment if the transcript level of at least ATF6B is elevated in the subject when compared to the transcript level in a control sample. In some aspects, the subject is treated with an ICI treatment if the transcript level of at least PAX8-AS1 is elevated in the subject when compared to the transcript level in a control sample. In some aspects, the subject is treated with an ICI treatment if the transcript level of at least RNVU1-19 is elevated in the subject when compared to the transcript level in a control sample. In some aspects, the subject is treated with an ICI treatment if the transcript level of at least VMO1 is elevated in the subject when compared to the transcript level in a control sample. In some aspects, the subject is treated with an ICI treatment if the transcript level of at least HBEGF is elevated in the subject when compared to the transcript level in a control sample. In some aspects, the subject is treated with an ICI treatment if the transcript level of at least CCDC144A is elevated in the subject when compared to the transcript level in a control sample. In some aspects, the subject is treated with an ICI treatment if the transcript level of at least SHISA8 is elevated in the subject when compared to the transcript level in a control sample. In some aspects, the subject is treated with an ICI treatment if the transcript level of at least NR4A2 is elevated in the subject when compared to the transcript level in a control sample. In some aspects, the subject is treated with an ICI treatment if the transcript level of at least PTGES is elevated in the subject when compared to the transcript level in a control sample. In some aspects, the subject is treated with an ICI treatment if the transcript level of at least SYN1 is elevated in the subject when compared to the transcript level in a control sample. In some aspects, the subject is treated with an ICI treatment if the transcript level of at least CXCL2 is elevated in the subject when compared to the transcript level in a control sample. In some aspects, the subject is treated with an ICI treatment if the transcript level of at least PMP22 is elevated in the subject when compared to the transcript level in a control sample. In some aspects, the subject is treated with an ICI treatment if the transcript level of at least CD83 is elevated in the subject when compared to the transcript level in a control sample. In some aspects, the subject is treated with an ICI treatment if the transcript level of at least EGR3 is elevated in the subject when compared to the transcript level in a control sample. In some aspects, the subject is treated with an ICI treatment if the transcript level of at least NUAK1 is elevated in the subject when compared to the transcript level in a control sample. In some aspects, the subject is treated with an ICI treatment if the transcript level of at least NOCT is elevated in the subject when compared to the transcript level in a control sample. In some aspects, the subject is treated with an ICI treatment if the transcript level of at least ATOH8 is elevated in the subject when compared to the transcript level in a control sample. In some aspects, the subject is treated with an ICI treatment if the transcript level of at least PLK2 is elevated in the subject when compared to the transcript level in a control sample. In some aspects, the subject is treated with an ICI treatment if the transcript level of at least ID1 is elevated in the subject when compared to the transcript level in a control sample. In some aspects, the subject is treated with an ICI treatment if the transcript level of at least ADRB1 is elevated in the subject when compared to the transcript level in a control sample. In some aspects, the subject is treated with an ICI treatment if the transcript level of at least SNAI1 is elevated in the subject when compared to the transcript level in a control sample. In some aspects, the subject is treated with an ICI treatment if the transcript level of at least NOTCH3 is elevated in the subject when compared to the transcript level in a control sample. In some aspects, the subject is treated with an ICI treatment if the transcript level of at least ATF3 is elevated in the subject when compared to the transcript level in a control sample. In some aspects, the subject is treated with an ICI treatment if the transcript level of at least DUSP2 is elevated in the subject when compared to the transcript level in a control sample. In some aspects, the subject is treated with an ICI treatment if the transcript level of at least PER1 is elevated in the subject when compared to the transcript level in a control sample. In some aspects, the subject is treated with an ICI treatment if the transcript level of at least TNFSF9 is elevated in the subject when compared to the transcript level in a control sample. In some aspects, the subject is treated with an ICI treatment if the transcript level of at least MAFF is elevated in the subject when compared to the transcript level in a control sample. In some aspects, the subject is treated with an ICI treatment if the transcript level of at least MI R4420 is elevated in the subject when compared to the transcript level in a control sample. In some aspects, the subject is treated with an ICI treatment if the transcript level of at least BRE-AS1 is elevated in the subject when compared to the transcript level in a control sample. In some aspects, the subject is treated with an ICI treatment if the transcript level of at least LOC102724428 is elevated in the subject when compared to the transcript level in a control sample. In some aspects, the subject is treated with an ICI treatment if the transcript level of at least GRASP is elevated in the subject when compared to the transcript level in a control sample. In some aspects, the subject is treated with an ICI treatment if the transcript level of at least TNFAIP3 is elevated in the subject when compared to the transcript level in a control sample. In some aspects, the subject is treated with an ICI treatment if the transcript level of at least TRIB1 is elevated in the subject when compared to the transcript level in a control sample. In some aspects, the subject is treated with an ICI treatment if the transcript level of at least PMAIP1 is elevated in the subject when compared to the transcript level in a control sample. In some aspects, the subject is treated with an ICI treatment if the transcript level of at least FAM46C is elevated in the subject when compared to the transcript level in a control sample. In some aspects, the subject is treated with an ICI treatment if the transcript level of at least NXT1 is elevated in the subject when compared to the transcript level in a control sample. In some aspects, the subject is treated with an ICI treatment if the transcript level of at least KLF9 is elevated in the subject when compared to the transcript level in a control sample.

In some aspects, the subject is treated with an ICI treatment if the transcript level of AREG is elevated prior to ICI treatment, compared to the transcript level in a control sample. In some aspects, the subject is treated with an ICI treatment if the transcript level of EREG is elevated prior to ICI treatment, compared to the transcript level in a control sample. In some aspects, the subject is treated with an ICI treatment if the transcript level of OSM is elevated prior to ICI treatment, compared to the transcript level in a control sample. In some aspects, the subject is treated with an ICI treatment if the transcript level of CSRNP1 is elevated prior to ICI treatment, compared to the transcript level in a control sample. In some aspects, the subject is treated with an ICI treatment if the transcript level of DDIT4 is elevated prior to ICI treatment, compared to the transcript level in a control sample. In some aspects, the subject is treated with an ICI treatment if the transcript level of IL-10 is elevated prior to ICI treatment, compared to the transcript level in a control sample. In some aspects, the subject is treated with an ICI treatment if the transcript level of PTGS2 is elevated prior to ICI treatment, compared to the transcript level in a control sample. In some aspects, the subject is treated with an ICI treatment if the transcript level of DUSP1 is elevated prior to ICI treatment, compared to the transcript level in a control sample. In some aspects, the subject is treated with an ICI treatment if the transcript level of CXCR4 is elevated prior to ICI treatment, compared to the transcript level in a control sample. In some aspects, the subject is treated with an ICI treatment if the transcript level of NIFL3 is elevated prior to ICI treatment, compared to the transcript level in a control sample. In some aspects, the subject is treated with an ICI treatment if the transcript level of FOS is elevated prior to ICI treatment, compared to the transcript level in a control sample. In some aspects, the subject is treated with an ICI treatment if the transcript level of NFKBIA is elevated prior to ICI treatment, compared to the transcript level in a control sample. In some aspects, the subject is treated with an ICI treatment if the transcript level of PPP1R15A is elevated prior to ICI treatment, compared to the transcript level in a control sample. In some aspects, the subject is treated with an ICI treatment if the transcript level of CD79A is elevated prior to ICI treatment, compared to the transcript level in a control sample. In some aspects, the subject is treated with an ICI treatment if the transcript level of JUNB is elevated prior to ICI treatment, compared to the transcript level in a control sample.

In some aspects, the subject is treated with an ICI treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C and/or GRASP are elevated in the subject when compared to the transcript levels in a control sample.

In some aspects, the subject is treated with an ICI treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B is lower prior to ICI treatment (baseline), compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are elevated in the subject when compared to the transcript levels in a control sample.

In some aspects, the subject is treated with an ICI treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment, compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are elevated in the subject when compared to the transcript levels in a control sample.

In some aspects, the subject is treated with an ICI treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to the transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are elevated in the subject when compared to the transcript levels in a control sample.

In some aspects, the subject is treated with an ICI treatment if the transcript levels of one or more of LILRB4, CISH, PARP9, RNF145, ASGR2, SLC16A13, LPAR6, GIMAP7, CXCR6, DHRS9, FCGR1CP, and/or ANKRD34B are lower prior to ICI treatment (baseline), compared to the transcript levels in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are elevated in the subject when compared to the transcript levels in a control sample.

In some aspects, the subject is treated with an ICI treatment if the transcript levels of one or more of LILRB4, CISH, and/or PARP9 are lower prior to ICI treatment (baseline), compared to the transcript levels in a control sample; and the transcript levels of one or more transcript of AREG, EREG, OSM, CSRNP1, DDIT4, IL-10, PTGS2, DUSP1, CXCR4, NFIL3, FOS, NFKBIA, PPP1R15A, CD79A, and/or JUNB are elevated in the subject when compared to the transcript levels in a control sample.

In some aspects, the subject is treated with an ICI treatment if the transcript levels of of LILRB4, CISH, and PARP9 is lower prior to ICI treatment (baseline), compared to the transcript levels in a control sample; and the transcript levels of AREG, EREG, OSM, CSRNP1, DDIT4, IL-10, PTGS2, DUSP1, CXCR4, NFIL3, FOS, NFKBIA, PPP1R15A, CD79A, and JUNB are elevated in the subject when compared to the transcript levels in a control sample.

In some aspects, the subject is treated with an ICI treatment if the transcript levels of one or more of LILRB4, GIMAP7, CISH, CXCR6, DHRS9, FCGR1CP, and/or ANKRD34B are lower prior to ICI treatment, compared to the transcript levels in a control sample; and the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, and/or LOC102724428 are elevated in the subject when compared to the transcript levels in a control sample.

In some aspects, the subject is treated with an ICI treatment if the transcript level of CISH is lower prior to ICI treatment, compared to the transcript level in a control sample; and the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are elevated in the subject when compared to the transcript levels in a control sample.

In some aspects, the subject is treated with an ICI treatment if the transcript levels of one or more of LILRB4, GIMAP7, CISH, and/or CXCR6 are lower prior to ICI treatment (baseline), compared to the transcript levels in a control sample; and the transcript levels of one or more of AREG, EREG, CXCL8, EGR1, G0S2, PAX8, ATF6B, PAX8-AS1, RNVU1-19, VMO1, HBEGF, CCDC144A, and/or SHISA8 are elevated in the subject when compared to the transcript levels in a control sample.

In some aspects, the subject is treated with an ICI treatment if the transcript level of PARP9 is lower prior to ICI treatment (baseline), compared to the transcript level in a control sample; and the transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are elevated in the subject when compared to the transcript levels in a control sample.

In some aspects, the subject is treated with an ICI treatment if the transcript levels of one or more of PARP9, CISH, CXCR6, LPAR6, and/or ASGR2 are lower prior to ICI treatment (baseline), compared to the transcript levels in a control sample; and the transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1 are elevated in the subject when compared to the transcript levels in a control sample.

In some aspects, the subject is treated with a non-ICI treatment, or an ICI treatment and an irAE mitigating treatment if the expression of one or more autoantibody Mi-2, GAD65, Myosin, Thyroglobulin and/or TPO or any combinations thereof, is elevated when compared to the expression in a control sample. In some aspects, the subject is treated with a non-ICI treatment, or an ICI treatment and an irAE mitigating treatment if the expression of autoantibody Mi-2 is elevated when compared to the expression in a control sample. In some aspects, the subject is treated with a non-ICI treatment, or an ICI treatment and an irAE mitigating treatment if the expression of autoantibody GAD65 is elevated when compared to the expression in a control sample. In some aspects, the subject is treated with a non-ICI treatment, or an ICI treatment and an irAE mitigating treatment if the expression of autoantibody Myosin is elevated when compared to the expression in a control sample. In some aspects, the subject is treated with a non-ICI treatment, or an ICI treatment and an irAE mitigating treatment if the expression of autoantibody Thyroglobulin is elevated when compared to the expression in a control sample. In some aspects, the subject is treated with a non-ICI treatment, or an ICI treatment and an irAE mitigating treatment if the expression of autoantibody TPO is elevated when compared to the expression in a control sample.

In some aspects, the subject is treated with an ICI treatment if the expression of the one or more of the autoantibody Mi-2, GAD65, Myosin, Thyroglobulin and/or TPO or any combinations thereof is lower compared to the expression in a control sample. In some aspects, the subject is treated with an ICI treatment if the expression of autoantibody Mi-2 is lower when compared to the expression in a control sample. In some aspects, the subject is treated with an ICI treatment if the expression of autoantibody GAD65 is lower when compared to the expression in a control sample. In some aspects, the subject is treated with an ICI treatment if the expression of autoantibody Myosin is lower when compared to the expression in a control sample. In some aspects, the subject is treated with an ICI treatment if the expression of autoantibody Thyroglobulin is lower when compared to the expression in a control sample. In some aspects, the subject is treated with an ICI treatment if the expression of autoantibody TPO is lower when compared to the expression in a control sample.

In some aspects, the subject is treated with a non-ICI treatment, or an ICI treatment and an irAE mitigating treatment if the expression of one or more cytokine CXCL2, CXCL5, CXCL6, CCL7, CCL19, IFNγ, IL-6, CXCL9, CXCL10 or any combinations thereof, is elevated when compared to the expression in a control sample. In some aspects the subject is treated with a non-ICI treatment, or an ICI treatment and an irAE mitigating treatment if the expression of cytokine CXCL2 is elevated when compared to the expression in a control sample. In some aspects, the subject is treated with a non-ICI treatment, or an ICI treatment and an irAE mitigating treatment if the expression of cytokine CXCL5 is elevated when compared to the expression in a control sample. In some aspects, the subject is treated with a non-ICI treatment, or an ICI treatment and an irAE mitigating treatment if the expression of cytokine CXCL6 is elevated when compared to the expression in a control sample. In some aspects, the subject is treated with a non-ICI treatment, or an ICI treatment and an irAE mitigating treatment if the expression of cytokine CCL7 is elevated when compared to the expression in a control sample. In some aspects, the subject is treated with a non-ICI treatment, or an ICI treatment and an irAE mitigating treatment if the expression of cytokine CCL19 is elevated when compared to the expression in a control sample. In some aspects, the subject is treated with a non-ICI treatment, or an ICI treatment and an irAE mitigating treatment if the expression of cytokine IFNγ is elevated when compared to the expression in a control sample. In some aspects, the subject is treated with a non-ICI treatment, or an ICI treatment and an irAE mitigating treatment if the expression of cytokine IL-6 is elevated when compared to the expression in a control sample. In some aspects, the subject is treated with a non-ICI treatment, or an ICI treatment and an irAE mitigating treatment if the expression of cytokine CXCL9 is elevated when compared to the expression in a control sample. In some aspects, the subject is treated with a non-ICI treatment, or an ICI treatment and an irAE mitigating treatment if the expression of cytokine CXCL10 is elevated when compared to the expression in a control sample.

In some aspects, the subject is treated with a non-ICI treatment, or an ICI treatment and an irAE mitigating treatment if the cytokine profile comprises a cytokine with sustained elevated expression after initiation of ICI treatment. In some aspects, the cytokine with sustained elevated expression after initiation of ICI treatment is one or more of CXCL5, IL-6, IFN-γ, CXCL9, CXCL10 or any combinations thereof. In some aspects, the cytokine with sustained elevated expression is CXCL5. In some aspects, the cytokine with sustained elevated expression is IL-6. In some aspects, the cytokine with sustained elevated expression is IFN-γ. In some aspects, the cytokine with sustained elevated expression is CXCL9. In some aspects, the cytokine with sustained elevated expression is CXCL10.

In some aspects, the subject is treated with an ICI treatment if the expression of one or more cytokine CXCL2, CXCL5, CXCL6, CCL7, CCL19, IFNγ, IL-6, CXCL9, CXCL10 or any combinations thereof, is lower when compared to the expression in a control sample. In some aspects, the subject is treated with an ICI treatment if the expression of cytokine CXCL2 is lower when compared to the expression in a control sample. In some aspects, the subject is treated with an ICI treatment if the expression of cytokine CXCL5 is lower when compared to the transcript level in a control sample. In some aspects, the subject is treated with an ICI treatment if the expression of cytokine CXCL6 is lower when compared to the expression in a control sample. In some aspects, the subject is treated with an ICI treatment if the expression of cytokine CCL7 is lower when compared to the expression in a control sample. In some aspects, the subject is treated with an ICI treatment if the expression of cytokine CCL19 is lower when compared to the expression in a control sample. In some aspects, the subject is treated with an ICI treatment if the expression of cytokine IFNγ is lower when compared to the expression in a control sample. In some aspects, the subject is treated with an ICI treatment if the expression of cytokine IL-6 is lower when compared to the expression in a control sample. In some aspects, the subject is treated with an ICI treatment if the expression of cytokine CXCL9 is lower when compared to the expression in a control sample. In some aspects, the subject is treated with an ICI treatment if the expression of cytokine CXCL10 is lower when compared to the expression in a control sample.

In some aspects, the subject is treated with a non-ICI treatment, or an ICI treatment and an irAE mitigating treatment if the abundance of one or more immune cell subset PD-L1+ naïve B cell, switched memory B cell or any combinations thereof, is lower and/or if the abundance of CTLA4+ monocyte is elevated when compared to the abundance in a control sample. In some aspects, the subject is treated with a non-ICI treatment, or an ICI treatment and an irAE mitigating treatment if the abundance of immune cell subset PD-L1+ naïve B cell is lower when compared to the abundance in a control sample. In some aspects, the subject is treated with a non-ICI treatment, or an ICI treatment and an irAE mitigating treatment if the abundance of immune cell subset switched memory B cell is lower when compared to the abundance in a control sample. In some aspects, the subject is treated with a non-ICI treatment, or an ICI treatment and an irAE mitigating treatment if the abundance of immune cell subset CTLA4+ monocyte is elevated when compared to the abundance in a control sample.

In some aspects, the subject is treated with an ICI treatment if the abundance of one or more immune cell subset PD-L1+ naïve B cell, switched memory B cell or any combinations thereof, is elevated and/or if the abundance of CTLA4+ monocyte is lower when compared to the abundance in a control sample. In some aspects, the subject is treated with an ICI treatment if the abundance of immune cell subset PD-L1+ naïve B cell is elevated when compared to the abundance in a control sample. In some aspects, the subject is treated with an ICI treatment if the abundance of immune cell subset switched memory B cell is elevated when compared to the abundance in a control sample. In some aspects, the subject is treated with an ICI treatment if the abundance of immune cell subset CTLA4+ monocyte is lower when compared to the abundance in a control sample.

In some aspects, the subject is changed to a non-ICI treatment, or an ICI treatment and an irAE mitigating treatment if the change in abundance of one or more immune cell subset PD-L1+ naïve B cell, switched memory B cell, and/or CXCR3+CD8 T cell is enhanced after initiation of ICI treatment in the sample from the subject, when compared to the abundance in a control sample. In some aspects, the subject is changed to a non-ICI treatment, or an ICI treatment and an irAE mitigating treatment if the change in the abundance of immune cell subset PD-L1+ naïve B cell is enhanced after initiation of ICI treatment when compared to the abundance in a control sample. In some aspects, the subject is changed to a non-ICI treatment, or an ICI treatment and an irAE mitigating treatment if the change in the abundance of immune cell subset switched memory B cell is enhanced after initiation of ICI treatment when compared to the abundance in a control sample. In some aspects, the subject is changed to a non-ICI treatment, or an ICI treatment and an irAE mitigating treatment if the change in the abundance of immune cell subset CXCR3+CD8 T cell is enhanced after initiation of ICI treatment when compared to the abundance in a control sample.

In some aspects, the subject is treated with a non-ICI treatment, or an ICI treatment and an irAE mitigating treatment if the transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or the transcript level, expression, or abundance of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C, GRASP, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or the transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or the transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if transcript level, expression, or abundance of one or more of LILRB4, CISH, PARP9, RNF145, ASGR2, SLC16A13, LPAR6, GIMAP7, CXCR6, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or the transcript level, expression, or abundance of one or more of AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte is higher and/or the transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell is lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte is higher and/or the level, expression or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte is higher and the level, expression or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or the transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of CISH, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte is higher and/or the transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte and/or the transcript level, expression, or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or the transcript level, expression, or abundance of one or more CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, TSC22D3, PD-L1+ naïve B cell, and/or switched memory B cell are lower, compared to the transcript level, expression, or abundance in a control sample, at baseline, or before initiation of ICI treatment.

In some aspects, the subject is treated with a non-ICI treatment, or an ICI treatment and an irAE mitigating treatment if the transcript level, expression, or abundance of one or more of PARP9, CISH, CXCR6, LPAR6, ASGR2, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are higher and/or the transcript level, expression, or abundance of one or more KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, EGR1 PD-L1+ naïve B cell, and/or switched memory B cell are lower, compared to the transcript level, expression, or abundance in a control sample, at baseline, or before initiation of ICI treatment.

In some aspects, the subject is treated with an ICI treatment if the transcript level, expression, or abundance of one or more of LILRB4, CISH, PARP9, RNF145, ASGR2, SLC16A13, LPAR6, GIMAP7, CXCR6, Mi-2, DHRS9, FCGR1CP, ANKRD34B, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or the transcript level, expression, or abundance of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C, GRASP and/or PD-L1+ naïve B cell are elevated, and/or switched memory B cell.

In some aspects, the subject is treated with an ICI treatment if the subject is treated with an ICI treatment if the transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or the transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, PD-L1+ naïve B cell, and/or switched memory B cell are higher, compared to the transcript level, expression, or abundance in a control sample, at baseline, or before initiation of ICI treatment.

In some aspects, the subject is treated with an ICI treatment if the transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or the transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are elevated, compared to the transcript level, expression, or abundance in a control sample, at baseline, or before initiation of ICI treatment.

In some aspects, the subject is treated with an ICI treatment if the transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte is lower and/or the transcript level, expression, or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell are elevated, compared to the transcript level, expression, or abundance in a control sample, at baseline, or before initiation of ICI treatment.

In some aspects, the subject is treated with an ICI treatment if the transcript level, expression, or abundance of one or more of LILRB4, CISH, PARP9, RNF145, ASGR2, SLC16A13, LPAR6, GIMAP7, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte is lower and/or the transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell is elevated, compared to the transcript level, expression, or abundance in a control sample, at baseline, or before initiation of ICI treatment.

In some aspects, the subject is treated with an ICI treatment if the transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or the transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are elevated, compared to the transcript level, expression, or abundance in a control sample, at baseline, or before initiation of ICI treatment.

In some aspects, the subject is treated with an ICI treatment if the transcript level, expression, or abundance of one or more of LILRB4, CISH, PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and the transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, PD-L1+ naïve B cell, and/or switched memory B cell are elevated, compared to the transcript level, expression, or abundance in a control sample, at baseline, or before initiation of ICI treatment.

In some aspects, the subject is treated with an ICI treatment if the transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or the transcript level, expression, or abundance of one or more AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, PD-L1+ naïve B cell, and/or switched memory B cell are elevated, compared to the transcript level, expression, or abundance in a control sample, at baseline, or before initiation of ICI treatment.

In some aspects, the subject is treated with an ICI treatment if the transcript level, expression, or abundance of one or more of CISH, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or the transcript level, expression, or abundance of one or more AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, KLF9, PD-L1+ naïve B cell, and/or switched memory B cell are higher, compared to the transcript level, expression, or abundance in a control sample, at baseline, or before initiation of ICI treatment.

In some aspects, the subject is treated with an ICI treatment if the transcript level, expression, or abundance of one or more of LILRB4, GIMAP7, CISH, CXCR6, DHRS9, FCGR1CP, ANKRD34B, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or the transcript level, expression, or abundance of one or more AREG, EREG, CXCL8, EGR1, G0S2, PAX8, ATF6B, PAX8-AS1, RNVU1-19, VMO1, HBEGF, CCDC144A, SHISA8, PD-L1+ naïve B cell, and/or switched memory B cell are elevated, compared to the transcript level, expression, or abundance in a control sample, at baseline, or before initiation of ICI treatment.

In some aspects, the subject is treated with an ICI treatment if the transcript level, expression, or abundance of one or more of PARP9, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte is lower and/or the transcript level, expression, or abundance of one or more CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, TSC22D3, PD-L1+ naïve B cell, and/or switched memory B cell are elevated, compared to the transcript level, expression, or abundance in a control sample, at baseline, or before initiation of ICI treatment.

In some aspects, the subject is treated with an ICI treatment if the transcript level, expression, or abundance of one or more of PARP9, CISH, CXCR6, LPAR6, ASGR2, Mi-2, GAD65, Myosin, Thyroglobulin, TPO, CXCL2, CXCL5, CXCL6, CCL7, CCL19, IFNγ, IL-6, CXCL9, CXCL10, and/or CTLA4+ monocyte are lower and/or the transcript level, expression, or abundance of one or more KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, EGR1 PD-L1+ naïve B cell, and/or switched memory B cell are higher, compared to the transcript level, expression, or abundance in a control sample, at baseline, or before initiation of ICI treatment.

In some aspects, the disclosed method for treatment using profile comprising immune cell, autoantibody, cytokine, or any combination thereof, can further comprise repeating one or more steps of the disclosed assessment and/or modifying one or more steps of the disclosed treatment. In an aspect, the disclosed method can further comprise modifying one or more of the administrations of the ICI compounds. In some aspects the method can comprise modifying one or more of steps of administration. For example, in an aspect, the method can be altered by changing the amount of one or more of the ICI compounds, thereof administered to a subject, or by changing the frequency of administration of one or more of the ICI compounds thereof to a subject, or by changing the duration of time one or more of the ICI compounds administered to a subject. In some aspects, the ICI compounds comprise one or more ICI compounds disclosed herein. In some aspects, the assessment using profile comprising immune cell, autoantibody, cytokine, or any combination thereof, can be repeated one or more times at the start of the treatment. In some aspects, the assessment using profile comprising immune cell, autoantibody, cytokine, or any combination thereof, can be repeated one or more times during the treatment. In some aspects, the assessment using profile comprising immune cell, autoantibody, cytokine, or any combination thereof, can be repeated one or more times after the treatment is completed.

In some aspects, the assessments can be automated using computer software analytical programs. The present disclosure provides computer implemented methods of detaining, comparing, and analyzing patterns of expression or levels of transcripts, autoantibodies, cytokines and/or immune cells, in order to assess the risk of irAE in a subject. The analytical programs can be interfaced with, for example, programs that are part of an automated transcripts, autoantibodies, cytokines and/or immune cell detection or quantification system so that data from the automated detection or quantification system can fed directly to the analytical programs. Computer implemented programs can be implemented to output, for example, the identity of transcripts, autoantibodies, cytokines and/or immune cell in the sample and the degree of upregulation or downregulation. The interface between the analytical programs may be direct or indirect. In some aspects, the programs of this disclosure can be designed to accept information on the detection or quantification of transcripts, autoantibodies, cytokines and/or immune cell, are able to implement data analysis, and output risk assessments. In some aspects the programs of disclosure can further output treatment strategies.

### III. Kits

The present disclosure provides kits for use in assessing risk of irAE and/or treating cancer, as described herein. Such kits can include one or more containers comprising ready-to-use microarray chips, or other detection devices, computer software data analysis for assessing risk and determining treatment strategies. In some aspects, kits can further include one or more containers comprising active agents for treatment of cancer or mitigating the toxicity.

Having described several aspects, it will be recognized by those skilled in the art that various modifications, alternative constructions, and equivalents may be used without departing from the spirit of the present disclosure. Additionally, a number of well-known processes and elements have not been described in order to avoid unnecessarily obscuring the present disclosure. Accordingly, this description should not be taken as limiting the scope of the present disclosure.

Those skilled in the art will appreciate that the presently disclosed aspects teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

### Patient, Materials, and Methods

### Clinical Data and Sample Collection

Cancer patients planned for ICI treatment were enrolled in a prospective biospecimen collection protocol approved by the UT Southwestern Institutional Review Board (IRB #STU 082015-053). Written, informed consent was obtained for collection of biospecimens and clinical data. Clinical, radiographic, and laboratory data were collected from the Epic electronic health record (Epic, Verona, Wisconsin). Because the diagnosis and characterization of irAE have been shown to be difficult, for each patient we determined the occurrence, timing, type, and severity of irAE was determined through adjudication of laboratory and ancillary data collected independently by two reviewers experienced in ICI administration and monitoring using established diagnostic criteria for myocarditis and myositis. After identifying myositis/myocarditis cases from this cohort, age- and gender-matched ICI-treated no toxicity cases were selected from the same cohort prior to analyzing any systemic immune parameters.

Peripheral blood samples were collected from all patients at pre-ICI baseline and approximately six weeks after ICI initiation. When feasible, sample collection was continued every three months while patients received ICI and sought to obtain samples at the time of clinically significant irAE, ideally prior to the initiation of steroids or other immunosuppressive agents.

Samples were centrifuged at 3000 rpm at 4° C. for 15 min to obtain plasma. Peripheral blood mononuclear cells (PBMC) were isolated using density gradient centrifugation in Ficoll-Paque PLUS Media following the manufacturer's instructions (Fisher Scientific, Waltham, MA).

### Cytokine/Chemokine Analysis

Cytokine and chemokine levels were determined with the Bio-Plex Pro Human Chemokine 40-plex Panel (Bio-Rad Laboratories, Hercules, California) using a Luminex 200 System. Bio-Plex Manager™ 6.1 software was used for data analysis. Cytokine and chemokine (pg/mL) concentrations were determined using a standard curve for mean fluorescence intensity versus pg/mL. As documented, these cytokines were stable over time in healthy controls not receiving ICI. Relevant publications were reviewed to identify cytokines that have been associated with inflammatory myopathies. Cytokines (a) included in the disclosed array (n=40), (b) identified in prior reports of inflammatory myopathies (n=34), and (c) included in the disclosed array and identified in prior reports (n=20) are shown in Table 1. Heatmaps were generated using Genesis cluster analysis of microarray data from the Thallinger Lab. Statistical analysis between the patients with irAE and NT control was done using Mann Whitney test or before and after ICI treatment groups using two-way ANOVA in GraphPad Prism 9.4.1.

TABLE 1
Cytokine panels

| Cytokine panel | Cytokines identified | Overlapping |
|---|---|---|
| in study | in literature | cytokines |
| (N = 40) | (N = 34) | (N = 20) |
| 6Ckine/CCL21 | 6Ckine/CCL21 | 6Ckine/CCL21 |
| GM-CSF | GM-CSF | GM-CSF |
| IFN-γ | IFN-γ | IFN-γ |
| IL-10 | IL-10 | IL-10 |
| IL-1β | IL-1β | IL-1β |
| IL-2 | IL-2 | IL-2 |
| IL-4 | IL-4 | IL-4 |
| IL-6 | IL-6 | IL-6 |
| IL-8/CXCL8 | IL-8/CXCL8 | IL-8/CXCL8 |
| IP-10/CXCL10 | IP-10/CXCL10 | IP-10/CXCL10 |
| MCP-1/CCL2 | MCP-1/CCL2 | MCP-1/CCL2 |
| MCP-2/CCL8 | MCP-2/CCL8 | MCP-2/CCL8 |
| MCP-4/CCL13 | MCP-4/CCL13 | MCP-4/CCL13 |
| MIG/CXCL9 | MIG/CXCL9 | MIG/CXCL9 |
| MIP-1a/CCL3 | MIP-1a/CCL3 | MIP-1a/CCL3 |
| TNF-α | TNF-α | TNF-α |
| I-TAC/CXCL11 | I-TAC/CXCL11 | I-TAC/CXCL11 |
| Eotaxin-1/CCL11 | Eotaxin | Eotaxin |
| Eotaxin-2/CCL24 | | |
| Eotaxin-3/CCL26 | | |
| ENA-78/CXCL5 | | |
| BCA-1/CXCL13 | | |
| CTACK/CCL27 | | |
| Fractalkine/CX3CL1 | | |
| GCP-2/CXCL6 | | |
| Gro-a/CXCL1 | | |
| Gro-b/CXCL2 | | |
| 1-309/CCL1 | | |
| IL-16 | | |
| MCP-3/CCL7 | | |
| MDC/CCL22 | | |
| MIF | | |
| MIP-1d/CCL15 | | |
| MIP-3a/CCL20 | | |
| MIP-3b/CCL19 | | |
| MPIF-1/CCL23 | | |
| SCYB16/CXCL16 | | |
| SDF-1a + b/CXCL12 | | |
| TARC/CCL17 | | |
| TECK/CCL25 | | |
| | CCL5 | |
| | MIP-1β | |
| | IL-1Ra | |
| | IL-5 | |
| | IL-7 | |
| | IL-9 | |
| | IL-12 | |
| | IL-13 | |
| | IL-15 | |
| | IL-17 | |
| | FGF basic | |
| | G-CSF | |
| | PDGF-BB | |
| | RANTES | |
| | VEGF | |
| | ATRN attractin | |

### Autoantigen Array Analysis

Plasma autoantibody profiling was perform using a previously developed custom protein array panel of 80 autoantigens, including nuclear antigens, cytosolic/matrix antigens, and 5 tissue/organ-specific antigens. Plasma samples were treated with Dnase-I to remove free DNA and then diluted 1:100 and hybridized with the arrays. IgG and IgM antibodies binding with the autoantigens were detected with cy3-conjugated anti-human IgG (Jackson ImmunoResearch Lab, 1:1000) and cy5-conjugated anti-human IgM (Jackson ImmunoResearch Lab, 1:1000). Arrays were scanned using Genepix 4400A scanner (Molecular Device) using wavelength 532 nm and 635 nm. Images were analyzed using Genepix 7.0 software. To avoid batch effect, all plasma samples were included in one assay and the NSI data were normalized for analysis. Statistical analysis between myositis cases and the controls of NT cases and healthy controls was done using unpaired student t test using non-parametric Mann Whitney test in GraphPad Prism 9.4.1.

Data analysis included the following pre-processing steps: (1) background subtraction and averaging of duplicated spots; (2) normalization of signal intensity of each antigen using internal controls across all samples; and (3) normalized signal intensity (NSI) for each autoantigen-autoantibody completed for each Genepix Report file generated per sample. NSI files were processed for downstream analysis using the Cluster and Treeview algorithm adopted from the Eisen Laboratory. Similar to cytokine analysis, autoantibodies associated with inflammatory myopathies were identified through literature review. Autoantigens (a) included in the disclosed array (n=80), (b) identified in prior reports of inflammatory myopathies (n=18), and (c) included in disclosed array and identified in prior reports (n=16) are shown in Table 2. Heatmaps were generated using Genesis cluster analysis of microarray data from the Thallinger Lab.

To avoid batch effects, all plasma samples were included in one assay and the NSI data were normalized. Statistical analyses between the patients with irAE and NT control were performed using a Mann Whitney test or before and after ICI treatment groups using two-way ANOVA in GraphPad Prism 9.4.1.

TABLE 2
Autoantibody panel

| Autoantibody panel | Autoantibodies | Overlapping |
|---|---|---|
| in study | identified in | autoantibodies |
| (N = 80) | literature (N = 18) | (N = 16) |
| Jo-1 | Jo-1 | Jo-1 |
| KU (P70/P80) | KU | KU (P70/P80) |
| MDA5 | MDA5 | MDA5 |
| Mi-2 | Mi-2 | Mi-2 |
| NXP2 | NXP2 | NXP2 |
| PL-12 | PL-12 | PL-12 |
| PL-7 | PL-7 | PL-7 |
| PM/Scl-100 | PM/Scl | PM/Scl-100 |
| PM/Scl-75 | | PM/Scl-75 |
| Ro/SS-A(52 kDa) | Ro52 | Ro/SS-A(52 kDa) |
| SAE1/SAE2 | SAE | SAE1/SAE2 |
| SRP54 | SRP | SRP54 |
| TIF1γ | TIF1γ | TIF1γ |
| U1-snRNP68/70 kDa | U1RNP | U1-snRNP68/70 kDa |
| U1-snRNP-A | | U1-snRNP-A |
| U1-snRNP-C | | U1-snRNP-C |
| Aggrecan | | |
| BCOADC-E2 | | |
| CENP-A | | |
| CENP-B | | |
| ComplementC3 | | |
| FibrinogenIV | | |
| FibrinogenTypeI-S | | |
| GAD65 | | |
| IA-2 | | |
| IF | | |
| LC1 | | |
| LKM 1 | | |
| M2 | | |
| MPO | | |
| Myosin | | |
| PDC-E2 | | |
| PR3 | | |
| Ro/SS-A(60 Kda) | | |
| Sm/RNP | | |
| Thyroglobulin | | |
| TPO | | |
| Vimentin | | |
| ComplementC1q | | |
| Gliadin | | |
| HSPG | | |
| Laminin | | |
| Proteoglycan | | |
| AGTR1 | | |
| BPI | | |
| Cardiolipin | | |
| CollagenIII | | |
| CollagenV | | |
| ComplementC4 | | |
| Cytochromec | | |
| DFS70 | | |
| dsDNA | | |
| Elastin | | |
| GBM | | |
| GenomicDNA | | |
| GP2 | | |
| GP210 | | |
| Histone | | |
| Human-CD40 | | |
| Insulin | | |
| KS | | |
| La/SS-B | | |
| MBP | | |
| Nucleolin | | |
| Nucleosome | | |
| Nup62 | | |
| OGDC-E2 | | |
| P0 | | |
| PCNA | | |
| Scl-70 | | |
| Sm | | |
| SmD | | |
| SmD1 | | |
| SP100 | | |
| β2-Glycoprotein 1 | | |
| ssDNA | | |
| TNF-alpha | | |
| tTG | | |
| U-snRNP-B/B′ | | |
| Vitronectin | | |
| | EJ | |
| | OJ | |
| | ARSa | |
| | cN1A | |
| | HMGCR | |

### Cytometry by Time of Flight (CyTOF)

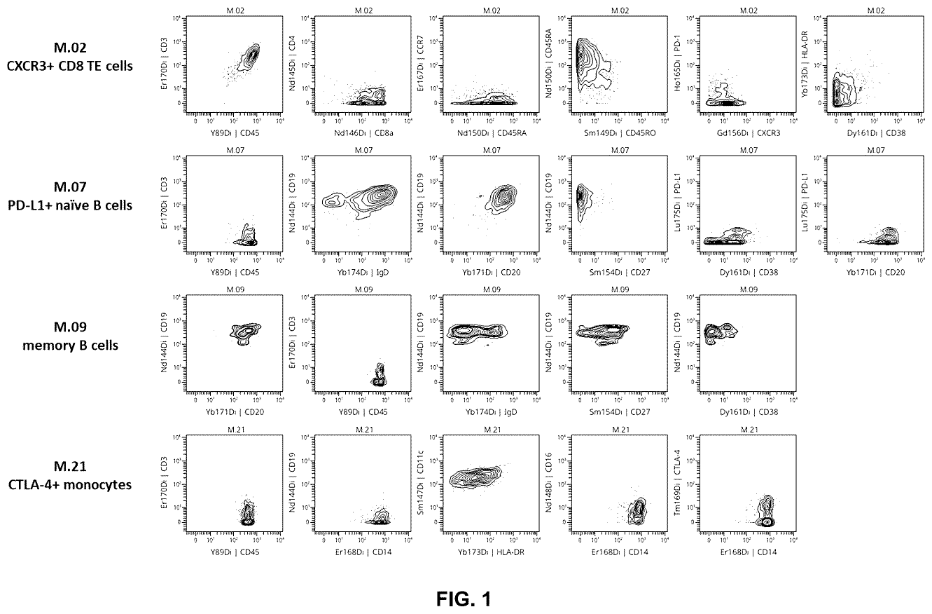

Cryopreserved PBMCs were thawed and stained with a panel of 40 antibodies (Table 3, metal isotope-labeled conjugates, Maxpar Direct Immune Profiling Assay Panel by Standard Biotools). Cells were analyzed on a Helios mass cytometer (Standard Biotools). Data were normalized and analyzed with gating on live cells using a cloud-based computational platform (OMIQ.ai, Insightful Science, San Diego, CA) following recommendations from Standard Biotool for use of Gaussian Discrimination Parameters. Using uniform manifold approximation and projection (UMAP) clustering analysis, distribution and expression characteristics of phenotypic markers were analyzed and compared across irAE and no toxicity control samples. Statistical analysis was conducted using EdgR and SAM at OMIQ on the abundance of clusters between the patient and the control samples. Cluster immune phenotypes were identified following standard immunophenotyping for the Human Immunology Project. The defining immune cell surface marker expression in noted clusters are shown in FIG. 1. The abundance of each cluster per sample was exported as percentage of total CD45+ cells and statistical analysis between the patients with irAE and NT control was done using Mann Whitney test or before and after ICI treatment groups using two-way ANOVA in GraphPad Prism 9.4.1.

TABLE 3
Antibodies used for CyTOF analysis.

| | Metal | |
|---|---|---|
| Probe/Target | Label | Antibody clone |
| CD45 | 89Y | HI30 |
| Cell viability Dye | 103Rh | |
| Normalization Bead | 140Ce | |
| CD196_CCR6 | 141Pr | G034E3 |
| b2-microglobulin | 142^{Nd} | 2M2 (Biolegend, Cat#316302) |
| CD123_IL-3R | 143Nd | 6H6 |
| CD19 | 144Nd | HIB19 |
| CD4 | 145Nd | RPA-T4 |
| CD8a | 146Nd | RPA-T8 |
| CD11c | 147Sm | Bu15 |
| CD16 | 148Nd | 3G8 |
| CD45RO | 149Sm | UCHL1 |
| CD45RA | 150Nd | HI100 |
| CD161 | 151Eu | HP-3G10 |
| CD194_CCR4 | 152Sm | L291H4 |
| CD25_IL-2Ra | 153Eu | BC96 |
| CD27 | 154Sm | O323 |
| CD57 | 155Gd | HNK-1 |
| CD183_CXCR3 | 156Gd | G025H7 |
| CD185_CXCR5 | 158Gd | J252D4 |
| Integrin_a4b7 | 159Tb | HU117 (R&D Systems, Cat#MAB10078) |
| CD28 | 160Gd | CD28.2 |
| CD38 | 161Dy | HB-7 |
| CCR9 | 162Dy | 112509 (R&D Systems, Cat#MAB179) |
| CD56_NCAM | 163Dy | NCAM16.2 |
| TCRgd | 164Dy | B1 |
| PD-1 | 165Ho | EH12.2H7 |
| CD294 | 166Er | BM16 |
| CD197_CCR7 | 167Er | G043H7 |
| CD14 | 168Er | 63D3 |
| CTLA-4 | 169Tm | BNI3 (Biolegend, Cat#369602) |
| CD3 | 170Er | UCHT1 |
| CD20 | 171Yb | 2H7 |
| CD66b | 172Yb | G10F5 |
| HLA-DR | 173Yb | LN3 |
| IgD | 174Yb | IA6-2 |
| PD-L1 | 175Lu | 29E.3A3 |
| CD127_IL-7Ra | 176Yb | A019D5 |
| DNA Intercalator 1 | 191Ir | |
| DNA Intercalator 2 | 193Ir | |

Unless indicated otherwise, antibody clones are from Standard Biotools.

### Bulk RNA-Seq Preparation and Analysis

Cryopreserved PBMCs from myositis/myocarditis irAE cases (N=4) and no toxicity controls (N=3) were thawed, and total RNA was extracted using RNeasy Mini kit (Qiagen, catalogue #74104). RNA-seq libraries were prepared using the Illumina TruSeq® Stranded mRNA Library prep kit (catalog #20020594) following the manufacturer's instruction. Barcoded RNA-seq libraries were sequenced on an Illumina NovaSeq 6000 sequencer platform using a (pair end) PE-150 sequencing protocol. Alignment, quantification and differential analysis were performed using the QBRC Bulk RNA-seq pipeline (https://github.com/QBRC/QBRC_BulkRnaSeqDE). Briefly, reads were aligned to a reference (GRCh38) with ‘STAR’ (v2.7.2b). Gene counts were quantified with ‘FeatureCounts’ (v1.6.4). Differential gene expression analysis was performed using the R package DEseq2′ (v1.26). The differential expressed genes with a false discovery rate (FDR)<0.05 were used to identify variances/clusters between the patient groups of irAE and no toxicity controls in heatmap with hierarchical clustering. Functional enrichment analysis for each variance/cluster was further analyzed using the publicly available website Enrichr for the top terms from GO_Biological_Process_2021 gene set library by gene otology analysis.

### Example 1. Clinical Features and Sample Information on the Patients and the Healthy Control

Out of 375 patients enrolled in the prospective ICI cohort, six cases (2%) were identified with a myositis and/or myocarditis irAE as follows: myositis (n=2), myocarditis (n=2), combined myositis-myocarditis (n=2). All six patients had received ICI targeting programmed death-1 (PD-1) (pembrolizumab or nivolumab) or PD-1 ligand (PD-L1) (durvalumab). Mean age was 66 years, and five of six patients were male. Additional clinical characteristics of these cases and six age- and gender-matched ICI-treated no toxicity controls are shown in Table 4.

All myositis/myocarditis irAE were National Cancer Institute (NCI) Common Terminology Criteria for Adverse Events CTCAE (version 5.0) grade in severity. Myositis irAE (with or without myocarditis) had relatively early onset (approximately 4 weeks after ICI initiation), while myocarditis-only irAE occurred later (one at 14 weeks, one at 60 weeks post-ICI initiation). One patient experienced diffuse muscle pain and weakness that rapidly improved with steroid administration. Serum creatine kinase (CK) was not elevated at the time, a phenomenon that has been described previously in a subset of patients with active myositis. One patient developed grade 5 myocarditis and grade 2 myositis around 4 weeks and died 31 days after ICI initiation. Otherwise, the myositis/myocarditis irAE cases treated with immunomodulatory therapy and no toxicity controls had similar survival (936 versus 993 days, respectively).

TABLE 4
Clinical features of patients with irAE and controls.

| | | | | irAE type/ | irAE | | | NT- | | |
|---|---|---|---|---|---|---|---|---|---|---|
| | Age | | | maximum | onset | CK | TnT | proBNP | EF | OS |
| Case | (years)/gender | Cancer type | ICI | grade | (days) | (U/L)* | (ng/dL)* | (pg/mL)* | (%)* | (days) |
| M1 | 70/M | Melanoma | PD-1 | Myocarditis/3 | 422 | | 37 | 9808 | 30 | 870 |
| M2 | 68/M | Pancreas | PD-1 | Myocarditis/3 | 99 | 2405 | 98 | 1670 | 35 | 430 |
| M3 | 55/F | Lung | PD-1 | Myositis/2 | 57 | 336 | 27 | 93 | | 942 |
| M4 | 69/M | Melanoma | PD-1 | Myositis/2 | 57 | 31 | | | | 1256 |
| M5 | 71/M | Lung | PD-L1 | Both/5 | 20 | 1674 | 4191 | 2054 | 45 | 31 |
| M6 | 65/M | Lung | PD-L1 | Both/3 | 28 | 9205 | 474 | 9205 | 55 | 1183 |
| NT1 | 66/M | Mesothelioma | PD-1 | | | | | | | 345 |
| NT2 | 73/M | Lung | PD-L1 | | | | | | | 1531 |
| NT3 | 74/M | Melanoma | PD-1 | | | | | | | 1320 |
| NT4 | 62/F | Lung | PD-1 | | | | | | | 733 |
| NT5 | 79/M | Melanoma | PD-1 | | | | | | | 1297 |
| NT6 | 66/M | Melanoma | PD-1 | | | | | | | 734 |

### Example 2: Comparison of Cytokine Profile Among Subjects

FIG. 2A-2K display cytokine parameters in myositis/myocarditis irAE cases and ICI-treated no toxicity controls. In general, no clear difference were observed in baseline, pre-treatment cytokines (FIG. 2A), with the exception of the single grade 5 (fatal) myositis/myocarditis case, which demonstrated higher baseline levels of C-X-C motif chemokine ligand 2 (CXCL2), CXCL5, CXCL6 C-C motif chemokine ligand 7 (CCL7), CCL19 IFNγ, and IL-6 (FIG. 2B-2H). After ICI initiation, the six myositis/myocarditis irAE cases sustained greater increases in CXCL9 (P=0.03, two-way ANOVA) and CXCL10 (P=0.12) than did the no toxicity controls (FIG. 21). These increases were most pronounced in the grade 5 myositis/myocarditis case (FIG. 2J-2K). Apart from the distinct cytokine features of the grade 5 case, clear differences according to irAE phenotype (myositis, myocarditis, overlap myositis/myocarditis) were not identified. A total of 20 cytokines included in the 40-cytokine panel had also been identified in reported cases of inflammatory myopathies. Among these, baseline CXCL5, IL-6, and, IFN-γ; CXCL9 and CXCL10 exhibited greater post-ICI increases, particularly among the single grade 5 case.

### Example 3: Comparison of Autoantibodies Among Subjects

Baseline autoantibody panels from irAE cases and no toxicity controls are shown in FIG. 3A. Those with significant differences according to occurrence and grade of myositis/myocarditis are shown in FIG. 3B-3F. For most of these (including Mi-2, an antibody associated with non-ICI-related inflammatory myopathies), the single case with grade 5 myocarditis drove the differences. No clear differences in autoantibody changes were observed after ICI initiation related to irAE development.

### Example 4: Comparison of Circulating Immune Cell Profile Among Subjects

For CyTOF analysis, equal sampling of 6,749 events per sample (the lowest common denominator across all samples) was performed, resulting in a total of 161,976 events across all 24 samples (two time-points per case). Based on distinct marker expression features, 28 clusters were identified representing major immune cell subsets including B cells, CD4 and CD8 T cells, monocytes, and NK cells from an overlaid data set of all files after UMAP clustering analysis (FIG. 4A). At pre-treatment baseline, compared to no toxicity controls, cases with myositis/myocarditis irAE had significantly lower levels of PD-L1+ naïve B cells (P=0.004), lower levels of switched memory B cells (P=0.03), and higher levels of CTLA4+ monocytes (P=0.03) (FIG. 4B). After ICI initiation, myositis/myocarditis cases had significantly greater increases in PD-L1+ naïve B cells (P=0.03), switched memory B cells (P=0.02), and CXCR3+CD8 T cells (P=0.01) (FIG. 4C). Four of 28 clusters (14%) showed distinct increases in irAE cases compared to the no toxicity cases (FIG. 4C).

### Example 5: Comparison of Transcription Profiles Among Subjects

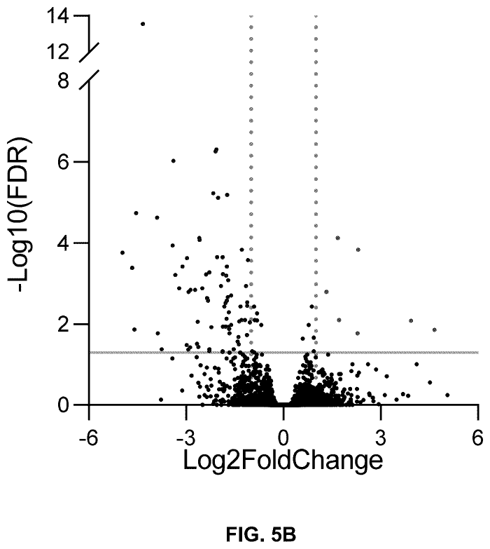

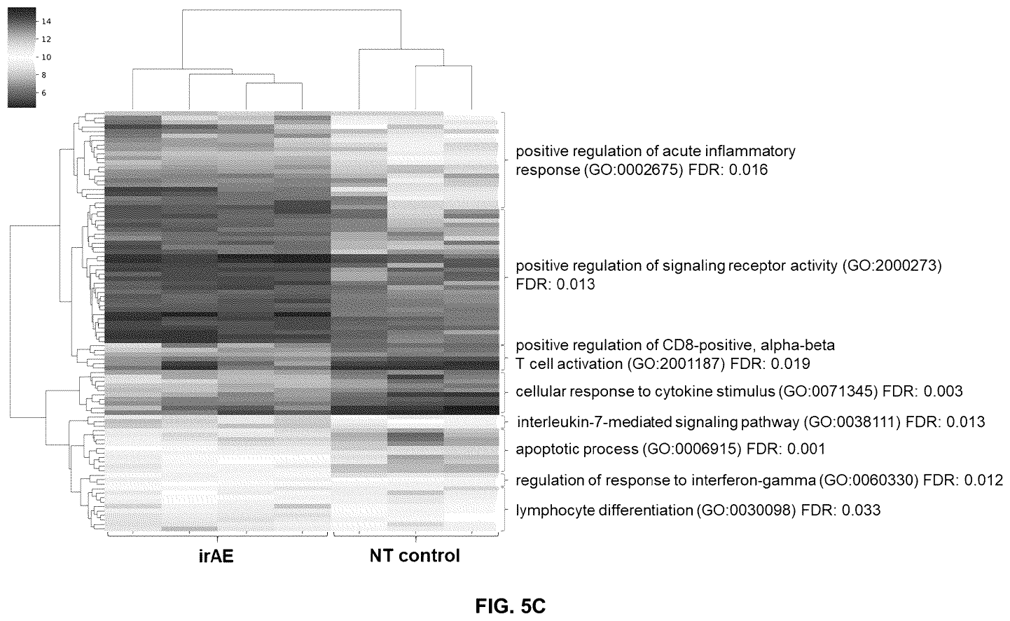

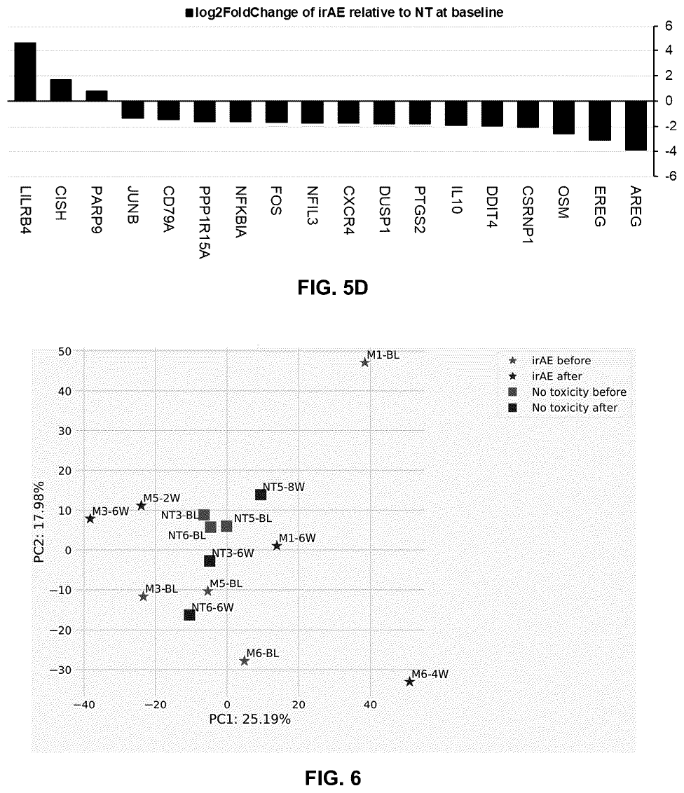

Prior to ICI initiation, transcription profiles of irAE cases and no toxicity control showed clear different profiles in Principal Component Analysis (PCA) plot, particularly in PCA1 axis (FIG. 5A). The differentially expressed genes (DEG) analysis showed 94 out of 14,174 genes with statistically significant (FDR<0.05) differences with 12 upregulated (Table 5), 82 downregulated (Table 6) between myositis/myocarditis cases and no toxicity controls. Among these were, 7 genes upregulated and 75 genes downregulated with |Log 2fold change (FC)|>1) (FIG. 5B). Gene otology (GO) analysis of the 94 genes with differential expression revealed 18 genes overlapping with 8 biological process clusters, including regulation of acute inflammatory response, regulation of CD8+ alpha-beta T cell activation, positive regulation of signaling receptor activity, cellular response to cytokine stimulus, and regulation of intracellular signal transduction (Table 7, FIG. 5C). The relative differences in expression of these 18 genes according between myositis/myocarditis cases and no toxicity controls are shown in FIG. 5D. Greatest differences were noted for upregulation in leukocyte immunoglobulin like receptor B4 (LILRB4, log 2FC=4.7), downregulation of Amphiregulin (AREG, log 2FC=−3.9), Epiregulin (EREG, log 2FC=−3.1), and Oncostatin M (OSM, log 2FC=−2.6). No clear differences in gene expression after ICI initiation between the two patient groups were observed (FIG. 6).

TABLE 5
Genes with increased RNA expression at baseline

| Gene Symbol | HGNC name | log2FoldChange | padj |
|---|---|---|---|
| RNF145 | ring finger protein 145 | 0.59 | 0.0231 |
| PARP9 | poly(ADP-ribose) polymerase family member 9 | 0.77 | 0.0106 |
| ASGR2 | asialoglycoprotein receptor 2 | 0.87 | 0.0037 |
| SLC16A13 | solute carrier family 16 member 13 | 0.93 | 0.0477 |
| LPAR6 | lysophosphatidic acid receptor 6 | 0.93 | 0.0216 |
| GIMAP7 | GTPase, IMAP family member 7 | 1.32 | 0.0016 |
| CISH | cytokine inducible SH2 containing protein | 1.67 | 0.0001 |
| CXCR6 | C-X-C motif chemokine receptor 6 | 1.71 | 0.0080 |
| DHRS9 | dehydrogenase/reductase 9 | 2.29 | 0.0173 |
| FCGR1CP | Fc gamma receptor Ic, pseudogene | 2.30 | 0.0001 |
| ANKRD34B | ankyrin repeat domain 34B | 3.93 | 0.0083 |
| LILRB4 | leukocyte immunoglobulin like receptor B4 | 4.65 | 0.0140 |

TABLE 6
Genes with decreased RNA expression at baseline

| Gene Symbol | HGNC name | log2FoldChange | padj |
|---|---|---|---|
| CXCL8 | C-X-C motif chemokine ligand 8 | −4.97 | 0.0002 |
| EGR1 | early growth response 1 | −4.67 | 0.0004 |
| G0S2 | G0/G1 switch 2 | −4.60 | 0.0137 |
| PAX8 | paired box 8 | −4.55 | 0.0000 |
| ATF6B | activating transcription factor 6 beta | −4.34 | 0.0000 |
| AREG | amphiregulin | −3.89 | 0.0000 |
| PAX8-AS1 | PAX8 antisense RNA 1 | −3.87 | 0.0173 |
| RNVU1-19 | RNA, variant U1 small nuclear 19 | −3.76 | 0.0421 |
| VMO1 | vitelline membrane outer layer 1 homolog | −3.42 | 0.0001 |
| HBEGF | heparin binding EGF like growth factor | −3.40 | 0.0000 |
| BRE-AS1 | BABAM2-AS1 (HGNC); BABAM2 antisense RNA 1 | −3.34 | 0.0006 |
| CCDC144A | coiled-coil domain containing 144A | −3.22 | 0.0013 |
| EREG | epiregulin | −3.12 | 0.0004 |
| SHISA8 | shisa family member 8 | −2.99 | 0.0335 |
| NR4A2 | nuclear receptor subfamily 4 group A member 2 | −2.98 | 0.0002 |
| PTGES | prostaglandin E synthase | −2.94 | 0.0434 |
| SYN1 | synapsin I | −2.93 | 0.0016 |
| CXCL2 | C-X-C motif chemokine ligand 2 | −2.88 | 0.0376 |
| PMP22 | peripheral myelin protein 22 | −2.87 | 0.0014 |
| CD83 | CD83 molecule | −2.73 | 0.0014 |
| EGR3 | early growth response 3 | −2.67 | 0.0310 |
| NUAK1 | NUAK family kinase 1 | −2.65 | 0.0088 |
| SLED1 | Not in HGNC; Proteoglycan 3, Pro Eosinophil Major | −2.63 | 0.0372 |
| | Basic Protein 2 Pseudogene | | |
| OSM | oncostatin M | −2.60 | 0.0001 |
| NOCT | nocturnin | −2.59 | 0.0001 |
| ATOH8 | atonal bHLH transcription factor 8 | −2.51 | 0.0013 |
| PLK2 | polo like kinase 2 | −2.39 | 0.0006 |
| ID1 | inhibitor of DNA binding 1 | −2.36 | 0.0023 |
| SNAI1 | snail family transcriptional repressor 1 | −2.33 | 0.0026 |
| ADRB1 | adrenoceptor beta 1 | −2.29 | 0.0421 |
| NOTCH3 | notch receptor 3 | −2.29 | 0.0477 |
| LOC102724428 | Not in HGNC; Salt Inducible Kinase 1B (Putative) | −2.29 | 0.0005 |
| ATF3 | activating transcription factor 3 | −2.23 | 0.0121 |
| DUSP2 | dual specificity phosphatase 2 | −2.17 | 0.0000 |
| GRASP | TAMALIN (HGNC); trafficking regulator and scaffold | −2.10 | 0.0000 |
| | protein tamalin | | |
| PER1 | period circadian regulator 1 | −2.07 | 0.0000 |
| TNFSF9 | TNF superfamily member 9 | −2.05 | 0.0002 |
| CSRNP1 | cysteine and serine rich nuclear protein 1 | −2.02 | 0.0000 |
| DDIT4 | DNA damage inducible transcript 4 | −1.93 | 0.0012 |
| MAFF | MAF bZIP transcription factor F | −1.88 | 0.0002 |
| MIR4420 | microRNA 4420 | −1.88 | 0.0492 |
| IL-10 | interleukin 10 | −1.88 | 0.0477 |
| GPX3 | glutathione peroxidase 3 | −1.87 | 0.0117 |
| TNFAIP3 | TNF alpha induced protein 3 | −1.87 | 0.0006 |
| KCNG1 | potassium voltage-gated channel modifier subfamily G | −1.82 | 0.0037 |
| | member 1 | | |
| PTGS2 | prostaglandin-endoperoxide synthase 2 | −1.79 | 0.0030 |
| AKAP5 | A-kinase anchoring protein 5 | −1.78 | 0.0117 |
| DUSP1 | dual specificity phosphatase 1 | −1.77 | 0.0006 |
| DGKK | diacylglycerol kinase kappa | −1.76 | 0.0075 |
| B4GALNT3 | beta-1,4-N-acetyl-galactosaminyltransferase 3 | −1.75 | 0.0026 |
| TRIB1 | tribbles pseudokinase 1 | −1.74 | 0.0004 |
| PMAIP1 | phorbol-12-myristate-13-acetate-induced protein 1 | −1.73 | 0.0000 |
| CXCR4 | C-X-C motif chemokine receptor 4 | −1.72 | 0.0022 |
| FAM46C | TENT5C (HGNC); terminal nucleotidyltransferase 5C | −1.71 | 0.0008 |
| TP53INP2 | tumor protein p53 inducible nuclear protein 2 | −1.71 | 0.0119 |
| NFIL3 | nuclear factor, interleukin 3 regulated | −1.69 | 0.0052 |
| DUSP4 | dual specificity phosphatase 4 | −1.68 | 0.0133 |
| FOS | Fos proto-oncogene, AP-1 transcription factor subunit | −1.67 | 0.0173 |
| NFKBIA | NFKB inhibitor alpha | −1.62 | 0.0106 |
| PPP1R15A | protein phosphatase 1 regulatory subunit 15A | −1.62 | 0.0020 |
| AVPI1 | arginine vasopressin induced 1 | −1.54 | 0.0421 |
| CD79A | CD79a molecule | −1.42 | 0.0273 |
| ARL4D | ADP ribosylation factor like GTPase 4D | −1.39 | 0.0210 |
| JCHAIN | joining chain of multimeric IgA and IgM | −1.38 | 0.0323 |
| BTG2 | BTG anti-proliferation factor 2 | −1.36 | 0.0087 |
| TLE1 | TLE family member 1, transcriptional corepressor | −1.33 | 0.0037 |
| JUNB | JunB proto-oncogene, AP-1 transcription factor subunit | −1.30 | 0.0081 |
| NXT1 | nuclear transport factor 2 like export factor 1 | −1.29 | 0.0001 |
| TOB1 | transducer of ERBB2, 1 | −1.19 | 0.0080 |
| PDE4D | phosphodiesterase 4D | −1.15 | 0.0012 |
| DNAJB1 | DnaJ heat shock protein family (Hsp40) member B1 | −1.13 | 0.0188 |
| ARID5B | AT-rich interaction domain 5B | −1.11 | 0.0036 |
| GPR153 | G protein-coupled receptor 153 | −1.11 | 0.0029 |
| KLF9 | KLF transcription factor 9 | −1.10 | 0.0003 |
| SBDS | SBDS ribosome maturation factor | −1.03 | 0.0092 |
| IER2 | immediate early response 2 | −0.97 | 0.0477 |
| TSC22D3 | TSC22 domain family member 3 | −0.91 | 0.0037 |
| JUND | JunD proto-oncogene, AP-1 transcription factor subunit | −0.90 | 0.0498 |
| GABARAPL1 | GABA type A receptor associated protein like 1 | −0.89 | 0.0080 |
| RUNX3 | RUNX family transcription factor 3 | −0.82 | 0.0082 |
| EIF1 | eukaryotic translation initiation factor 1 | −0.81 | 0.0055 |
| JOSD1 | Josephin domain containing 1 | −0.69 | 0.0106 |

TABLE 7
Gene ontology processes associated with clusters
by differentially expressed genes (DEG) analysis.

| | | | Overlapped | P | Adjusted | Odds | Combined |
|---|---|---|---|---|---|---|---|
| Cluster | Gene | Term | genes | value | P value | Ratio | Score |
| 1 | CD83 | Positive | OSM | 9.90E−05 | 0.016 | 170.7 | 1573.6 |
| | NR4A2 | regulation of | PTGS2 | | | | |
| | HBEGF | acute | | | | | |
| | G0S2 | inflammatory | | | | | |
| | EGR1 | response | | | | | |
| | LOC102724428 | (GO: 0002675) | | | | | |
| | OSM | | | | | | |
| | PTGS2 | | | | | | |
| | TP53INP2 | | | | | | |
| | NXT1 | | | | | | |
| | GRASP | | | | | | |
| | PMAIP1 | | | | | | |
| | GPR153 | | | | | | |
| | TLE1 | | | | | | |
| | DUSP4 | | | | | | |
| | ATF3 | | | | | | |
| | MAFF | | | | | | |
| | PAX8-AS1 | | | | | | |
| | CXCL8 | | | | | | |
| | VMO1 | | | | | | |
| 2 | PAX8 | Positive | IL-10 | 2.79E−05 | 0.013 | 60.7 | 636.1 |
| | ATF6B | regulation of | AREG | | | | |
| | AREG | signaling | EREG | | | | |
| | RNVU1-19 | receptor | | | | | |
| | BRE-AS1 | activity | | | | | |
| | CCDC144A | (GO: 2000273) | | | | | |
| | EREG | | | | | | |
| | SHISA8 | | | | | | |
| | PTGES | | | | | | |
| | SYN1 | | | | | | |
| | CXCL2 | | | | | | |
| | PMP22 | | | | | | |
| | EGR3 | | | | | | |
| | NUAK1 | | | | | | |
| | SLED1 | | | | | | |
| | NOCT | | | | | | |
| | ATOH8 | | | | | | |
| | PLK2 | | | | | | |
| | ID1 | | | | | | |
| | SNAI1 | | | | | | |
| | ADRB1 | | | | | | |
| | NOTCH3 | | | | | | |
| | TNFSF9 | | | | | | |
| | MIR4420 | | | | | | |
| | IL-10 | | | | | | |
| | GPX3 | | | | | | |
| | KCNG1 | | | | | | |
| | AKAP5 | | | | | | |
| | DGKK | | | | | | |
| | B4GALNT3 | | | | | | |
| | AVPI1 | | | | | | |
| | ARL4D | | | | | | |
| 3 | DHRS9 | Positive | LILRB4 | 2.70E−03 | 0.019 | 499.7 | 2955.7 |
| | CXCR6 | regulation of | | | | | |
| | SLC16A13 | CD8-positive, | | | | | |
| | FCGR1CP | alpha-beta T | | | | | |
| | ANKRD34B | cell activation | | | | | |
| | LILRB4 | (GO: 2001187) | | | | | |
| 4 | RUNX3 | Cellular | DUSP1 | 6.23E−05 | 0.003 | 27.2 | 263.5 |
| | CXCR4 | response to | CXCR4 | | | | |
| | BTG2 | cytokine | FOS | | | | |
| | IER2 | stimulus | JUNB | | | | |
| | JUND | (GO: 0071345) | | | | | |
| | DUSP1 | | | | | | |
| | TSC22D3 | | | | | | |
| | EIF1 | | | | | | |
| | FOS | | | | | | |
| | JUNB | | | | | | |
| 5 | CISH | Interleukin-7- | CISH | 2.85E−03 | 0.013 | 555.0 | 3252.9 |
| | GIMAP7 | mediated | | | | | |
| | LPAR6 | signaling | | | | | |
| | | pathway | | | | | |
| | | (GO: 0038111) | | | | | |
| 6 | DNAJB1 | Apoptotic | NFKBIA | 3.45E−06 | 0.001 | 58.0 | 730.0 |
| | TNFAIP3 | process | PPP1R15A | | | | |
| | NFKBIA | (GO: 0006915) | CSRNP1 | | | | |
| | PPP1R15A | | DDIT4 | | | | |
| | TOB1 | | | | | | |
| | JOSD1 | | | | | | |
| | DUSP2 | | | | | | |
| | CSRNP1 | | | | | | |
| | DDIT4 | | | | | | |
| | PER1 | | | | | | |
| 7 | ASGR2 | Regulation | PARP9 | 2.10E−03 | 0.012 | 768.6 | 4739.7 |
| | PARP9 | of response | | | | | |
| | RNF145 | to interferon- | | | | | |
| | | gamma | | | | | |
| | | (GO: 0060330) | | | | | |
| 8 | JCHAIN | Lymphocyte | CD79A | 7.67E−04 | 0.033 | 60.7 | 435.3 |
| | FAM46C | differentiation | NFIL3 | | | | |
| | KLF9 | (GO: 0030098) | | | | | |
| | ARID5B | | | | | | |
| | CD79A | | | | | | |
| | GABARAPL1 | | | | | | |
| | PDE4D | | | | | | |
| | SBDS | | | | | | |
| | TRIB1 | | | | | | |
| | NFIL3 | | | | | | |

Further analysis was conducted to identify genes with highest fold change (increase or decrease) at baseline, compared to no toxicity controls. Table 8 shows genes selected based on<1.0 log 2foldchange or >1.0 log 2foldchange. In patients with toxicity (myositis), twenty-seven genes which were decreased at baseline measurement and one gene (CISH) which was increased at baseline measurement, were determined to have lowest false discovery rate (Table 9). Further, GO (Gene Ontology) Consortium's webtool for biological process enrichment was queried across the genes with both decreased and increased RNA transcripts (http://geneontology.org/). The category of genes associated with “regulation of immune process” had a p=1.04E-05 and a false discovery rate of 1.62E-03; wherein a subset of 19 genes were identified (Table 10). The category of genes associated with “response to stimulus” had a p=2.35E-11 and a false discovery rate of 1.23E-07; wherein a subset of 57 genes were identified (Table 11). Individual and combined RNA transcript expression in a baseline peripheral blood sample disclosed herein could be used to establish the probability of future toxicity. Even though, the present study was directed to myositis, some or all of these expressed RNAs could be used as markers for a wide range of immune related adverse events.

TABLE 8
genes with the highest fold decrease or increase at baseline
in samples associated with toxicity. Genes selected based
on <−1.0 log2foldchange or >1.0 log2foldchange

| | | |
|---|---|---|
| Gene Symbol | HGNC name | log2FoldChange |

DECREASED RNA TRANSCRIPTS

| | | |
|---|---|---|
| CXCL8 | C-X-C motif chemokine ligand | −4.97 |
| | 8 | |
| EGR1 | early growth response 1 | −4.67 |
| G0S2 | G0/G1 switch 2 | −4.60 |
| PAX8 | paired box 8 | −4.55 |
| ATF6B | activating transcription factor | −4.34 |
| | 6 beta | |
| AREG | amphiregulin | −3.89 |
| PAX8-AS1 | PAX8 antisense RNA 1 | −3.87 |
| RNVU1-19 | RNA, variant U1 small nuclear | −3.76 |
| | 19 | |
| VMO1 | vitelline membrane outer layer | −3.42 |
| | 1 homolog | |
| HBEGF | heparin binding EGF like | −3.40 |
| | growth factor | |
| BRE-AS1 | BABAM2-AS1(HGNC); | −3.34 |
| | BABAM2 antisense RNA 1 | |
| CCDC144A | coiled-coil domain containing | −3.22 |
| | 144A | |
| EREG | epiregulin | −3.12 |
| SHISA8 | shisa family member 8 | −2.99 |
| NR4A2 | nuclear receptor subfamily 4 | −2.98 |
| | group A member 2 | |
| PTGES | prostaglandin E synthase | −2.94 |
| SYN1 | synapsin I | −2.93 |
| CXCL2 | C-X-C motif chemokine ligand | −2.88 |
| | 2 | |
| PMP22 | peripheral myelin protein 22 | −2.87 |
| CD83 | CD83 molecule | −2.73 |
| EGR3 | early growth response 3 | −2.67 |
| NUAK1 | NUAK family kinase 1 | −2.65 |
| SLED1 | Not in HGNC; Proteoglycan 3, | −2.63 |
| | Pro Eosinophil Major Basic | |
| | Protein 2 Pseudogene | |
| OSM | oncostatin M | −2.60 |
| NOCT | nocturnin | −2.59 |
| ATOH8 | atonal bHLH transcription | −2.51 |
| | factor 8 | |
| PLK2 | polo like kinase 2 | −2.39 |
| ID1 | inhibitor of DNA binding 1 | −2.36 |
| SNAI1 | snail family transcriptional | −2.33 |
| | repressor 1 | |
| ADRB1 | adrenoceptor beta 1 | −2.29 |
| NOTCH3 | notch receptor 3 | −2.29 |
| LOC102724428 | Not in HGNC; Salt Inducible | −2.29 |
| | Kinase 1B (Putative) | |
| ATF3 | activating transcription factor | −2.23 |
| | 3 | |
| DUSP2 | dual specificity phosphatase 2 | −2.17 |
| GRASP | TAMALIN; trafficking regulator | −2.10 |
| | and scaffold protein tamalin | |
| PER1 | period circadian regulator 1 | −2.07 |
| TNFSF9 | TNF superfamily member 9 | −2.05 |
| CSRNP1 | cysteine and serine rich | −2.02 |
| | nuclear protein 1 | |
| DDIT4 | DNA damage inducible | −1.93 |
| | transcript 4 | |
| MAFF | MAF bZIP transcription factor | −1.88 |
| | F | |
| MIR4420 | microRNA 4420 | −1.88 |
| IL-10 | interleukin 10 | −1.88 |
| GPX3 | glutathione peroxidase 3 | −1.87 |
| TNFAIP3 | TNF alpha induced protein 3 | −1.87 |
| KCNG1 | potassium voltage-gated | −1.82 |
| | channel modifier subfamily G | |
| | member 1 | |
| PTGS2 | prostaglandin-endoperoxide | −1.79 |
| | synthase 2 | |
| AKAP5 | A-kinase anchoring protein 5 | −1.78 |
| DUSP1 | dual specificity phosphatase 1 | −1.77 |
| DGKK | diacylglycerol kinase kappa | −1.76 |
| B4GALNT3 | beta-1,4-N-acetyl- | −1.75 |
| | galactosaminyltransferase 3 | |
| TRIB1 | tribbles pseudokinase 1 | −1.74 |
| PMAIP1 | phorbol-12-myristate-13- | −1.73 |
| | acetate-induced protein 1 | |
| CXCR4 | C-X-C motif chemokine | −1.72 |
| | receptor 4 | |
| FAM46C | TENT5C; terminal | −1.71 |
| | nucleotidyltransferase 5C | |
| TP53INP2 | tumor protein p53 inducible | −1.71 |
| | nuclear protein 2 | |
| NFIL3 | nuclear factor, interleukin 3 | −1.69 |
| | regulated | |
| DUSP4 | dual specificity phosphatase 4 | −1.68 |
| FOS | Fos proto-oncogene, AP-1 | −1.67 |
| | transcription factor subunit | |
| NFKBIA | NFKB inhibitor alpha | −1.62 |
| PPP1R15A | protein phosphatase 1 | −1.62 |
| | regulatory subunit 15A | |
| AVPI1 | arginine vasopressin induced | −1.54 |
| | 1 | |
| CD79A | CD79a molecule | −1.42 |
| ARL4D | ADP ribosylation factor like | −1.39 |
| | GTPase 4D | |
| JCHAIN | joining chain of multimeric IgA | −1.38 |
| | and IgM | |
| BTG2 | BTG anti-proliferation factor 2 | −1.36 |
| TLE1 | TLE family member 1, | −1.33 |
| | transcriptional corepressor | |
| JUNB | JunB proto-oncogene, AP-1 | −1.30 |
| | transcription factor subunit | |
| NXT1 | nuclear transport factor 2 like | −1.29 |
| | export factor 1 | |
| TOB1 | transducer of ERBB2, 1 | −1.19 |
| PDE4D | phosphodiesterase 4D | −1.15 |
| DNAJB1 | DnaJ heat shock protein | −1.13 |
| | family (Hsp40) member B1 | |
| ARID5B | AT-rich interaction domain 5B | −1.11 |
| GPR153 | G protein-coupled receptor | −1.11 |
| | 153 | |
| KLF9 | KLF transcription factor 9 | −1.10 |
| SBDS | SBDS ribosome maturation | −1.03 |
| | factor | |

INCREASED RNA TRANSCRIPTS

| | | |
|---|---|---|
| GIMAP7 | GTPase, IMAP family | 1.32 |
| | member 7 | |
| CISH | cytokine inducible SH2 | 1.67 |
| | containing protein | |
| CXCR6 | C-X-C motif chemokine | 1.71 |
| | receptor 6 | |
| DHRS9 | dehydrogenase/reductase 9 | 2.29 |
| FCGR1CP | Fc gamma receptor lc, | 2.30 |
| | pseudogene | |
| ANKRD34B | ankyrin repeat domain 34B | 3.93 |
| LILRB4 | leukocyte immunoglobulin like | 4.65 |
| | receptor B4 | |

TABLE 9
Genes with the lowest false discovery rate

| Gene | log2FoldChange |
|---|---|
| CXCL8 | −4.97 |
| EGR1 | −4.67 |
| PAX8 | −4.55 |
| ATF6B | −4.34 |
| AREG | −3.89 |
| VMO1 | −3.42 |
| HBEGF | −3.40 |
| BRE-AS1 | −3.34 |
| EREG | −3.12 |
| NR4A2 | −2.98 |
| OSM | −2.60 |
| NOCT | −2.59 |
| PLK2 | −2.39 |
| LOC102724428 | −2.29 |
| DUSP2 | −2.17 |
| GRASP | −2.10 |
| PER1 | −2.07 |
| TNFSF9 | −2.05 |
| CSRNP1 | −2.02 |
| MAFF | −1.88 |
| TNFAIP3 | −1.87 |
| DUSP1 | −1.77 |
| TRIB1 | −1.74 |
| PMAIP1 | −1.73 |
| FAM46C | −1.71 |
| NXT1 | −1.29 |
| KLF9 | −1.10 |
| CISH | 1.67 |

TABLE 10
genes highly associated with the immune system

| Genes | Change by RNAseq at baseline |
|---|---|
| CD79A | Decreased |
| CD83 | Decreased |
| PLK2 | Decreased |
| PARP9 | Increased |
| PDE4D | Decreased |
| TRIB1 | Decreased |
| EREG | Decreased |
| CXCL8 | Decreased |
| EGR3 | Decreased |
| JUNB | Decreased |
| DUSP1 | Decreased |
| NFKBIA | Decreased |
| TNFSF9 | Decreased |
| TNFAIP3 | Decreased |
| IL-10 | Decreased |
| JUND | Decreased |
| FOS | Decreased |
| RUNX3 | Decreased |
| TSC22D3 | Decreased |

TABLE 11
genes highly associated with biological response to stimuli

| Genes | Change by RNAseq at baseline |
|---|---|
| KLF9 | Decreased |
| CXCR4 | Decreased |
| ATF6B | Decreased |
| CD79A | Decreased |
| CD83 | Decreased |
| PLK2 | Decreased |
| GRASP | Decreased |
| PPP1R15A | Decreased |
| GPX3 | Decreased |
| PARP9 | Increased |
| BTG2 | Decreased |
| PTGS2 | Decreased |
| NOTCH3 | Decreased |
| CISH | Increased |
| AKAP5 | Decreased |
| PDE4D | Decreased |
| SBDS | Decreased |
| TRIB1 | Decreased |
| SNAI1 | Decreased |
| PAX8 | Decreased |
| NFIL3 | Decreased |
| EREG | Decreased |
| AREG | Decreased |
| DGKK | Decreased |
| PTGES | Decreased |
| ASGR2 | Increased |
| CSRNP1 | Decreased |
| DNAJB1 | Decreased |
| CXCL8 | Decreased |
| OSM | Decreased |
| ARID5B | Decreased |
| ID1 | Decreased |
| NR4A2 | Decreased |
| EGR3 | Decreased |
| JUNB | Decreased |
| TLE1 | Decreased |
| CXCL2 | Decreased |
| DUSP1 | Decreased |
| GPR153 | Decreased |
| G0S2 | Decreased |
| ATF3 | Decreased |
| NFKBIA | Decreased |
| TNFSF9 | Decreased |
| NUAK1 | Decreased |
| ATOH8 | Decreased |
| TNFAIP3 | Decreased |
| ADRB1 | Decreased |
| HBEGF | Decreased |
| IL-10 | Decreased |
| JUND | Decreased |
| FOS | Decreased |
| GABARAPL1 | Decreased |
| CXCR6 | Increased |
| LPAR6 | Increased |
| PMAIP1 | Decreased |
| DDIT4 | Decreased |
| EGR1 | Decreased |

### SUMMARY OF EXAMPLES

Myositis and myocarditis represent uncommon but potentially severe irAE arising in patients receiving ICI. To understand the underlying biology and to identify potential biomarkers, systemic immune parameters including serologic markers reported in spontaneous inflammatory myopathies were analyzed at pre-treatment baseline and shortly after ICI initiation in six patients with myositis, myocarditis, or myositis+myocarditis. These findings were compared to age- and gender-matched ICI-treated patients who did not develop irAE.

From a clinical perspective, myositis/myocarditis was quite rare, representing 2% of our ICI-treated cohort. Similarly, meta-analyses and reviews of international registries have demonstrated rates of 1-3%, although rates of reporting appear to be increasing. All patients in the present study had received anti-PD1/PDL1 therapies. Although myositis and myocarditis have been reported with both anti-PD1/PDL1 as well as anti-CTLA4 ICI, multi-center series have identified a greater association with PD1/PDL1-directed and combination treatments. Findings disclosed here may also reflect the predominance of these treatments in the cohort. The median onset of myositis at around 30 days after ICI initiation, while clearly earlier than most irAE, approximates the timing reported previously.

Systemic immune parameters in these cases only partially resembled those reported in non-ICI-related inflammatory myopathies. In particular, pre-treatment levels of autoantibodies such as Mi-2 and Myosin overlapped with the no toxicity controls except for the single fatal irAE case. Furthermore, no difference was observed in the levels of other antibodies associated with non-ICI myositis, such as PL7, PL12, MDA5, and Jot. This suggested that ICI-related myositis/myocarditis appeared to join other irAE such as ICI-related hepatitis and colitis, which feature only partial overlap with their non-ICI-related correlates.

The observation that IFNγ-inducible cytokines and chemokines involved in T cell activation and recruitment (e.g., CXCL9, CXCL10) sustained greater post-ICI increases in irAE cases may underlie the greater post-treatment increases seen in CD8+ T cells myositis/myocarditis cases. It also echoes observations from multiple prior studies of irAE. The finding that the greatest levels of inflammatory cytokines at baseline and post-ICI initiation were noted in the case with the most severe (in this case, grade 5) irAE is highly relevant, as the avoidance of lethal autoimmune toxicities represents a central priority for the field of immune-oncology.

The lower baseline levels but greater post-treatment increases in these key inflammatory cytokines and cellular populations suggested a state of immune dysregulation in patients at greater irAE risk. This was further supported by RNA sequencing analysis, which revealed lower baseline levels of genes associated with both positive and negative inflammatory responses in myositis/myocarditis cases compared to no toxicity controls. Another clinical scenario supporting a link between immune dysregulation and autoimmunity is HIV/AIDS, in which up to 60% of patients may develop rheumatologic conditions, particularly during periods of immune reconstitution.

This study included a detailed approach to collection and adjudication of clinical data, the availability of longitudinal blood samples, and a large cohort capable of identifying a small number of myositis/myocarditis cases. In summary, ICI-related myositis/myocarditis are extremely rare irAE, but with a potentially fulminant course and high mortality. As has been observed with other irAE, their serologic features appear to differ from comparable conditions not related to ICI. Immune dysregulation data may allow predict these toxicities in the future. Identification of potential predictors and biologic features of myositis and other rare irAE, could guide ICI treatment, providing better prognosis for the patients.

What is claimed is:

1. A method of treating an immune-related adverse events (irAE) associated with immune checkpoint inhibitor (ICI) treatment in a subject comprising:
a) providing a sample from the subject;
b) assessing the level of one or more transcript in the sample; and
c) predicting risk for developing/diagnosing irAE in the subject wherein, the subject is predicted as having a high risk of developing or diagnosed with having irAE if:
i. the transcript levels of one or more of leukocyte immunoglobulin like receptor B4 (LILRB4), cytokine inducible SH2 containing protein (CISH), poly(ADP-ribose) polymerase family member 9 (PARP9), ring finger protein 145 (RNF145), asialoglycoprotein receptor 2 (ASGR2), solute carrier family 16 member 13 (SLC16A13), lysophosphatidic acid receptor 6 (LPAR6), GTPase, IMAP family member 7 (GIMAP7), and/or C-X-C motif chemokine receptor 6 (CXCR6) are elevated prior to ICI treatment (baseline), compared to the transcript levels in a control sample; and/or
ii. if the transcript levels of one or more of Amphiregulin (AREG), epiregulin (EREG), Oncostatin M (OSM), cysteine and serine rich nuclear protein 1 (CSRNP1), DNA damage inducible transcript 4 (DDIT4), IL-10 (interleukin 10), Prostaglandin-endoperoxide synthase (PTGS2), Dual Specificity Phosphatase 1 (DUSP1), C-X-C chemokine receptor type 4 (CXCR4), Nuclear Factor, Interleukin 3 Regulated (NFIL3), Fos proto-oncogene, AP-1 transcription factor subunit (FOS), NFKB inhibitor alpha (NFKBIA), PPP1R15A (protein phosphatase 1 regulatory subunit 15A), CD79A, JunB proto-oncogene, AP-1 transcription factor subunit (JUNB), C-X-C motif chemokine ligand 8 (CXCL8), Early growth response 1 (EGR1), G0/G1 switch 2 (G0S2), paired box 8 (PAX8), activating transcription factor 6 beta (ATF6B), PAX8 antisense RNA1 (PAX8-AS1), RNA, variant U1 small nuclear 19 (RNVU1-19), vitelline membrane outer layer 1 homolog (VMO1), heparin binding EGF like growth factor (HBEGF), coiled-coil domain containing 144A (CCDC144A), shisa family member 8 (SHISA8), nuclear receptor subfamily 4 group A member 2 (NR4A2), prostaglandin E synthase (PTGES), synapsin I (SYN1), C-X-C motif chemokine ligand 2 (CXCL2), Peripheral myelin protein 22 (PMP22), CD83, early growth response 3 (EGR3), NUAK family kinase 1 (NUAK1), nocturnin (NOCT), atonal bHLH transcription factor 8 (ATOH8), polo like kinase (PLK2), inhibitor of DNA binding 1 (ID1), adrenoceptor beta 1 (ADRB1), snail family transcriptional repressor 1 (SNAI1), notch receptor 3 (NOTCH3), activating transcription factor 3 (ATF3), dual specificity phosphatase 2 (DUSP2), period circadian regulator 1 (PER1), TNF superfamily member 9 (TNFSF9), MAF bzip transcription factor F (MAFF), microRNA 4420 (MI R4420), glutathione peroxidase (GPX3), TNF alpha induced protein 3 (TNFAIP3), potassium voltage-gated channel modifier subfamily G member 1 (KCNG1), prostaglandin-endoperoxidase synthase 2 (PTGS2), A-kinase anchoring protein 5 (AKAP5), dual specificity phosphatase 1 (DUSP1), diacylglycerol kinase kappa (DGKK), beta-1,4,-N-acetyl-galactosaminyltransferase 3 (B4GALNT3), tribbles pseudokinase 1 (TRIB1), phorobol-12-myristate-13-acetate-induced protein 1 (PMAIP1), C-X-C motif chemokine receptor 4 (CXCR4), tumor protein p53 inducible nuclear protein 2 (TP531NP2), nuclear factor, interleukin 3 regulated (NFIL3), dual specificity phosphatase 4 (DUSP4), NFKB inhibitor alpha (NFKBIA), arginine vasopressin induced 1 (AVPI1), CD79a, ADP ribosylation factor like GTPase 4D (ARL4D), joining chain of multimeric IgA and IgM (JCHAIN), BTG anti-proliferation factor 2 (BTG2), TLE family member 1, transcriptional corepressor (TLE1), nuclear transport factor 2 like export factor 1 (NXT1), transducer of ERBB2, 1 (TOB1), phosphodiesterase 4D (PDE4D), DNAJ heat shocj protein family member B1 (DNAJB1), AT-rich interaction domain 5B (ARID5B), G protein-coupled receptor 153 (GPR153), KLF transcription factor 9 (KLF9), SBDS ribosome maturation factor (SBDS), immediate early response 2 (IER2), TSC22 domain family member 3 (TSC22D3), GABA type A receptor associated protein like 1 (GABARAPL1), JunD proto-oncogene, AP-1 transcription factor subunit (JUND), RUNX family transcription factor 3 (RUNX3), BABAM2 antisense RNA 1 (BRE-AS1), putative salt inducible kinas 1B (LOC102724428), FAM46C (FAM46C), and/or general receptor for phosphoinositides 1-associated scaffold protein (GRASP) are lower in the subject when compared to the transcript levels in a control sample
d) treating the subject with:
(i) an ICI therapy if subject is diagnosed as low risk of developing irAE;
(ii) a non-ICI therapy if the subject is diagnosed as high risk of developing irAE; or
(iii) an ICI therapy and an irAE mitigating therapy if the subject is diagnosed as high risk of developing irAE.

2. The method of claim 1, wherein the irAE comprises ICI-related myositis, ICI-related myocarditis, or ICI-related myositis and myocarditis.

3. The method of claim 1, wherein the assessment of transcript levels is performed before ICI treatment.

4. The method of claim 1, wherein the subject is predicted as high risk of developing irAE if:
a) the transcript levels of one or more of LILRB4, CISH, and/or PARP9 are elevated prior to ICI treatment (baseline), compared to the transcript levels in a control sample; and/or
b) the transcript levels of one or more of LILRB4, CISH, GIMAP7, and/or CXCR6, DHRS9, FCGR1CP, and/or ANKRD34B are elevated prior to ICI treatment (baseline), compared to the transcript levels in a control sample.

5. The method of claim 1, wherein the subject is predicted as high risk of developing irAE if:
a) the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP are lower prior to ICI treatment, compared to the transcript levels in a control sample;
b) the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are lower prior to ICI treatment, compared to the transcript levels control sample;
c) the transcript levels of one or more of AREG, EREG, CXCL8, EGR1, G0S2, PAX8, ATF6B, PAX8-AS1, RNVU1-19, VMO1, HBEGF, CCDC144A, and/or SHISA8, are lower prior to ICI treatment, compared to the transcript levels in a control sample; and/or
d) the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower prior to ICI treatment, compared to the transcript levels in a control sample.

6. The method of claim 1, wherein the subject is predicted as having a high risk of developing irAE if:
a) the transcript level of PARP9 is elevated prior to ICI treatment (baseline), compared to the transcript level in a control sample; and/or
b) the transcript levels of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are lower prior to ICI treatment (baseline), compared to the transcript levels in a control sample.

7. The method of claim 1, wherein the subject is predicted as high risk for irAE if
a) the transcript levels of one or more of PARP9, CISH, CXCR6, LPAR6, and/or ASGR2 are elevated prior to ICI treatment (baseline), compared to the transcript levels in a control sample; and/or
b) the transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1 are lower prior to ICI treatment (baseline), compared to the transcript levels in a control sample.

8. The method of claim 1, wherein the sample is whole blood, serum, plasma, cerebrospinal fluid, pleural fluid, pericardial fluid, peritoneal fluid, bone marrow, tissue, urine, cerebrospinal fluid (CSF), or other body fluid, and wherein the control sample is procured from a subject with low risk of developing irAE.

9. The method of claim 1, wherein assessing transcript levels (step b) comprises RNA-seq, Nanopore sequencing, Nanostring, multiplex RT-PCR, single-plex RT-PCR, NASBA, Fluorescence measurements, or spectrophotometry.

10. The method of claim 1, wherein the transcript levels are relative transcript levels.

11. The method of claim 1, wherein the method further comprises assessing the if the expression of one or more of Mi-2, GAD65, Myosin, Thyroglobulin, and/or TPO are elevated in the sample from the subject compared to the expression in a control sample.

12. The method of claim 1, wherein the method further comprises assessing if the expression of one or more of CXCL2, CXCL5, CXCL6, CCL7, CCL19, IFNγ, IL-6, CXCL9, and/or CXCL10 are elevated in the sample from the subject compared to the expression in a control sample.

13. The method of claim 1, wherein the method further comprises assessing if the abundance of one or more immune cells PD-L+ naive B cells, and/or switched memory B cells are decreased, and/or if immune cell CTLA-4+ monocyte is elevated, in the sample from the subject compared to the control sample.

14. The method of claim 1, further comprising repeating steps (a)-(c) at a second time point, thereby permitting determination of a change in the subject's risk of developing irAE and/or diagnosis of irAE in the sample from the subject compared to a control sample.

15. The method of claim 1, further comprising predicting the subject as having low risk if:
a) the transcript levels of one or more of LILRB4, CISH, PARP9, RNF145, ASGR2, SLC16A13, LPAR6, GIMAP7, and/or CXCR6 prior to ICI treatment (baseline) are lower or equivalent compared to the transcript levels in a control sample; and/or
b) the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP53INP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C, and/or GRASP are elevated or equivalent during ICI treatment, compared to the transcript levels in a control sample.

16. A method of treating a subject with cancer comprising:
(a) providing a sample from the subject;
(b) assessing the level of one or more transcripts in the sample;
(c) predicting the subject's risk of developing irAE, wherein the subject is diagnosed as:
i. low risk when the transcript levels of one or more of LILRB4, CISH, PARP9, RNF145, ASGR2, SLC16A13, LPAR6, GIMAP7, and/or CXCR6 are lower or equal to the transcript levels in a control sample;
ii. low risk when the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP531NP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C, and/or GRASP are elevated prior to ICI treatment (baseline) or equal to the transcript levels in a control sample;
iii. high risk when the transcript levels of one or more of LILRB4, CISH, PARP9, RNF145, ASGR2, SLC16A13, LPAR6, GIMAP7, and/or CXCR6 is elevated than the transcript levels in a control sample; and/or
iv. high risk when the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, GPX3, TNFAIP3, KCNG1, PTGS2, AKAP5, DUSP1, DGKK, B4GALNT3, TRIB1, PMAIP1, CXCR4, TP531NP2, NFIL3, DUSP4, NFKBIA, AVPI1, CD79a, ARL4D, JCHAIN, BTG2, TLE1, NXT1, TOB1, PDE4D, DNAJB1, ARID5B, GPR153, KLF9, SBDS, IER2, TSC22D3, JUND, GABARAPL1, RUNX3, BRE-AS1, LOC102724428, FAM46C, and/or GRASP are lower than the transcript levels in a control sample; and
(d) treating the subject with:
(i) an ICI therapy if subject is diagnosed as low risk of developing irAE;
(ii) a non-ICI therapy if the subject is diagnosed as high risk of developing irAE; or
(iii) an ICI therapy and an irAE mitigating therapy if the subject is diagnosed as high risk of developing irAE.

17. The method of claim 16, wherein irAE comprises ICI-related myositis, ICI-related myocarditis, or ICI-related myositis and myocarditis.

18. The method of claim 16, wherein the subject is predicted as high risk of developing irAE if:
a) the transcript levels of one or more of LILRB4, CISH, and/or PARP9 are elevated prior to ICI treatment (baseline), compared to the transcript levels in a control sample; and/or
b) if the transcript levels of one or more of LILRB4, CISH, GIMAP7, and/or CXCR6 are elevated prior to ICI treatment (baseline), compared to the transcript levels in a control sample.

19. The method of claim 16, wherein the subject is predicted as high risk of developing irAE if:
a) the transcript levels of one or more of AREG, EREG, OSM, CSRNP1, DDIT4, IL-10, PTGS2, DUSP1, CXCR4, NFIL3, FOS, NFKBIA, PPP1R15A, CD79A, JUNB, CXCL8, EGR1, G0S2, PAX8, ATF6B, PAX8-AS1, RNVU1-19, VMO1, HBEGF, CCDC144A, SHISA8, NR4A2, PTGES, SYN1, CXCL2, PMP22, CD83, EGR3, NUAK1, NOCT, ATOH8, PLK2, ID1, ADRB1, SNAI1, NOTCH3, ATF3, DUSP2, PER1, TNFSF9, MAFF, MIR4420, BRE-AS1, LOC102724428, and/or GRASP are lower prior to ICI treatment, compared to the transcript levels in a control sample
b) if the transcript levels of one or more of AREG, EREG, OSM, CSRNP1, DDIT4, IL-10, PTGS2, DUSP1, CXCR4, NFIL3, FOS, NFKBIA, PPP1R15A, CD79A, and/or JUNB are lower prior to ICI treatment, compared to the transcript levels in a control sample;
c) the transcript levels of one or more of AREG, EREG, CXCL8, EGR1, G0S2, PAX8, ATF6B, PAX8-AS1, RNVU1-19, VMO1, HBEGF, CCDC144A, and/or SHISA8, are lower prior ICI treatment, compared to the transcript levels in a control sample; and/or
d) the transcript levels of one or more of AREG, EREG, CXCL8, EGR1, PAX8, ATF6B, VMO1, HBEGF, BRE-AS1, NR4A2, OSM, NOCT, PLK2, LOC102724428, DUSP2, GRASP, PER1, TNFSF9, CSRNP1, MAFF, TNFAIP3, DUSP1, TRIB1, PMAIP1, FAM46C, NXT1, and/or KLF9 are lower prior to ICI treatment, compared to the transcript levels in a control sample.

20. The method of claim 16, wherein the subject is predicted as high risk for irAE if:
a) the transcript level of PARP9 is elevated prior to ICI treatment (baseline), compared to the transcript level in a control sample; and/or
b) the transcript level of one or more of CD79A, CD83, PLK2, PDE4D, TR1B1, EREG, CXCL8, EGR3, JUNB, DUSP1, NFKBIA, TNFSF9, TNFAIP3, IL-10, JUND, FOS, RUNX3, and/or TSC22D3 are lower prior to ICI treatment (baseline), compared to the transcript levels in a control sample.

21. The method of claim 16, wherein the subject is predicted as high risk for irAE if:
a) the transcript levels of one or more of PARP9, CISH, CXCR6, LPAR6, and/or ASGR2 are elevated prior to ICI treatment (baseline), compared to the transcript levels in a control sample; and/or
b) the transcript levels of one or more of KLF9, CXCR4, ATF6B, CD79A, CD83, PLK2, GRASP, PPP1R15A, GPX3, PARP9, BTG2, PTGS2, NOTCH3, AKAP5, PDE4D, SBDS, TRIB1, SNAI1, PAX8, NFIL3, EREG, AREG, DGKK, PTGES, CSRNP1, DNAJB1, CXCL8, OSM, ARID5B, ID1, NR4A2, EGR3, JUNB, TLE1, CXCL2, DUSP1, GPR153, G0S2, ATF3, NFKBIA, TNFS9, NUAK1, ATOH8, TNFAIP3, ADRB1, HBEGF, IL-10, JUND, FOS, GABARAPL1, PMAIP1, DDIT4, and/or EGR1 are lower prior to ICI treatment (baseline), compared to the transcript levels in a control sample.

22. The method of claim 16, wherein the method further comprises assessing if the expression of one or more of CXCL2, CXCL5, CXCL6, CCL7, CCL19, IFNγ, IL-6, CXCL9, and/or CXCL10 are elevated in the sample from the subject compared to the expression in a control sample.

23. The method of claim 16, wherein the method further comprises assessing if the expression of one or more Mi-2, GAD65, Myosin, Thyroglobulin and/or TPO are elevated in the sample from the subject compared to the expression in a control sample.

24. The method of claim 16, wherein the method further comprises assessing if the abundance of one or more wherein the assessment comprises identifying if the abundance of one or more of PD-L+ naive B cells, and/or switched memory B cells are decreased, and/or if the abundance of CTLA-4+ monocytes are elevated in the sample from the sample from the subject compared to the abundance in a control sample.

25. The method of claim 16, wherein the control sample is procured from a subject with low risk of developing irAE.

26. The method of claim 16, wherein the transcript levels are relative transcript levels.
